(19) United States
(12) Patent Application Publication
(10) Pub. No.: US 2012/0041009 A1
(43) Pub. Date: Feb. 16, 2012

(54) PYRIMIDINE COMPOUND AND ITS USE IN PEST CONTROL

(75) Inventor: Hajime Mizuno, Toyonaka-shi (JP)

(73) Assignee: SUMITOMO CHEMICAL COMPANY, LIMITED, Chuo-ku, Tokyo (JP)

(21) Appl. No.: 13/265,177

(22) PCT Filed: Mar. 11, 2010
(86) PCT No.: PCT/JP2010/058249

§ 371 Date: Oct. 19, 2011

(30) Foreign Application Priority Data
May 18, 2009 (JP) 2009-119585

(51) Int. Cl. A01N 43/836; C07D 413/14; A01P 7/04; A01P 7/02; A01P 5/00; C07D 413/04; A01P 7/00

(52) U.S. Cl. 514/274; 544/333; 514/256; 544/316; 544/328; 544/319

(57) ABSTRACT

A pyrimidine compound represented by below formula (I) has excellent control activity against pests and is useful as an active ingredient of a pest controlling agent.

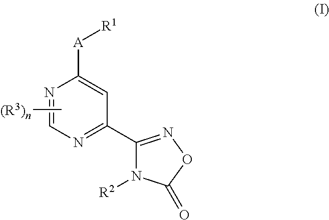

### TECHNICAL FIELD

The present invention relates to a pyrimidine compound and its use in pest control.

### BACKGROUND ART

Heretofore, many compounds have been developed and used for controlling pests. For example, US2002/0013326 discloses that a certain pyrimidine compound is effective to control pests.

### DISCLOSURE OF THE INVENTION

However, since these pyridine compounds do not necessarily have sufficient control activity against pests, it has been required to develop a compound having excellent control activity against pests

The present inventors have intensively studied so as to find a compound having excellent effect of controlling pests and found that a compound represented by formula (I) shown below has excellent control activity against pests, thus leading to the present invention.

The present invention provides as follows.
[1] A pyrimidine compound represented by formula (I):

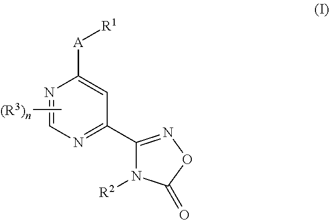

wherein R¹ represents a C1-C7 haloalkyl group optionally substituted with one or more members selected from Group γ, a phenyl group optionally substituted with one or more members selected from Group β, or a pyridyl group optionally substituted with one or more members selected from Group β;
A represents a single bond, oxygen, sulfur, —N(R¹⁰)—, —CH₂—, or —CH₂O—;
wherein R¹⁰ represents a C1-C7 alkyl group optionally substituted with halogen, a C3-C7 alkenyl group optionally substituted with halogen, a C3-C7 alkynyl group optionally substituted with halogen, a C2-C7 alkoxyalkyl group, a cyanomethyl group, or hydrogen;
R² represents a C1-C7 alkyl group optionally substituted with halogen, a (C3-C7 cycloalkyl)methyl group optionally substituted with one or more members selected from Group α, a benzyl group optionally substituted with one or more members selected from Group β, a cyanomethyl group hydrogen, or
any one of the following groups Q¹ to Q⁵:

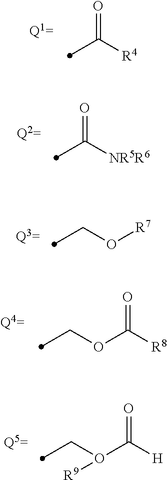

wherein R⁴ represents a C1-C7 alkyl group optionally substituted with halogen, a C3-C7 cycloalkyl group optionally substituted with one or more members selected from Group α, or hydrogen,
R⁵ represents a C1-C7 alkyl group optionally substituted with halogen, a C3-C7 alkenyl group optionally substituted with halogen, a C1-C7 alkyloxy group optionally substituted with halogen, or a C3-C7 cycloalkyl group optionally substituted with one or more members selected from Group α,
R⁶ represents a C1-C7 alkyl group optionally substituted with halogen, a C3-C7 alkenyl group optionally substituted with halogen, a C3-C7 cycloalkyl group optionally substituted with one or more members selected from Group α,
or hydrogen, or
—NR⁵R⁶ is replaced by a pyrrolidin-1-yl group optionally substituted with one or more members selected from Group α, a piperidino group optionally substituted with one or more members selected from Group α, a hexamethyleneimin-1-yl group optionally substituted with one or more members selected from Group α, a morpholino group optionally substituted with one or more members selected from Group α, or a thiomorpholin-4-yl group optionally substituted with one or more members selected from Group α,
R⁷ represents a C1-C7 alkyl group optionally substituted with halogen, a phenyl group optionally substituted with one or more members selected from Group β, a benzyl group optionally substituted with one or more members selected from Group β, or a C3-C7 cycloalkyl group optionally substituted with one or more members selected from Group α,
R⁸ represents a C1-C7 alkyl group optionally substituted with halogen, a phenyl group optionally substituted with one or more members selected from Group β, or a C3-C7 cycloalkyl group optionally substituted with one or more members selected from Group α, and
R⁹ represents a C1-C7 alkyl group optionally substituted with halogen, or hydrogen;
R³ represents a C1-C7 alkyl group optionally substituted with halogen, a C1-C7 alkylthio group optionally substituted with halogen, a C1-C7 alkyloxy group optionally substituted with halogen, a C3-C7 cycloalkyl group optionally substituted with one or more members selected from Group α, a C3-C7 cycloalkyloxy group optionally substituted with one or more members selected from Group α, or halogen; and
n represents an integer of 0 to 2 and, when n is 2, each R³ is the same or different:
Group γ: a group consisting of a C1-C3 alkyloxy group optionally substituted with halogen, a C3-C7 alkenyloxy group optionally substituted with halogen, a C3-C7 alkynyloxy group optionally substituted with halogen, and a tri(C1-C4 alkyl)silyloxy group and a hydroxy group;
Group α: a group consisting of a C1-C7 alkyl group optionally substituted with halogen, and halogen; and
Group β: a group consisting of a C1-C7 alkyl group optionally substituted with halogen, a C1-C7 alkyloxy group optionally substituted with halogen, halogen, a cyano group, and a nitro group (hereinafter referred to as the present compound).
[2] The pyrimidine compound according to [1], wherein R² is hydrogen.
[3] The pyrimidine compound according to [1], wherein R² is Q¹.
[4] The pyrimidine compound according to [1], wherein R² is Q².
[5] The pyrimidine compound according to [1], wherein R² is Q³.
[6] The pyrimidine compound according to [1], wherein R² is Q⁴.
[7] The pyrimidine compound according to any one of [1] to [6], wherein R¹ is a C1-C7 haloalkyl group optionally substituted with one or more members selected from Group γ;
[8] The pyrimidine compound according to any one of [1] to [6], wherein R¹ is a phenyl group optionally substituted with one or more members selected from Group β, or a pyridyl group optionally substituted with one or more members selected from Group β.
[9] A pest controlling agent comprising the pyrimidine compound according to any one of [1] to [8] and an inert carrier.
[10] A method for controlling pests, which comprises applying an effective amount of the pyrimidine compound according to any one of [1] to [8] to pests or habitats of pests.
[11] Use of the pyrimidine compound according to any one of [1] to [8] for control of pests.

### MODE OF CARRYING OUT THE INVENTION

In the present invention, halogen means fluorine, chlorine, bromine or iodine.

Examples of the “C1-C7 haloalkyl group optionally substituted with one or more members selected from Group γ” represented by R¹ include “C1-C7 haloalkyl groups” such as a fluoromethyl group, a chloromethyl group, a bromomethyl group, a difluoromethyl group, a dichloromethyl group, a dibromomethyl group, a trifluoromethyl group, a trichloromethyl group, a dichlorofluoromethyl group, a chlorodifluoromethyl group, a bromodifluoromethyl group, a 2,2,2-trifluoroethyl group, a 1,1,2,2,2-pentafluoroethyl group, a 3,3,3-trifluoropropyl group, a 2,2,3,3,3-pentafluoropropyl group, a heptafluoropropyl group, a 2,2,2-trifluoro-1-methylethyl group, a 2,2,2-trifluoro-1-ethylethyl group, a 2,2,3,3,3-pentafluoro-1-methylpropyl group, a 2,2,3,3,3-pentafluoro-1-ethylpropyl group, a 2,2,2-trifluoro-1-(trifluoromethyl)ethyl group, and a heptafluoroisopropyl group; and
“C1-C7 haloalkyl groups substituted with one or more members selected from the group consisting of a C1-C3 alkyloxy group optionally substituted with halogen, a C3-C7 alkynyloxy group optionally substituted with halogen, a C3-C7 alkynyloxy group optionally substituted with halogen, a tri(C1-C4 alkyl)silyloxy group, and a hydroxy group” such as a 2,2,2-trifluoro-1-hydroxyethyl group, a 2,2,2,3,3-pentafluoro-1-hydroxypropyl group, a 2,2,2-trifluoro-1-hydroxy-1-methylethyl group, a 2,2,2-trifluoro-1,1-dihydroxyethyl group, a 2,2,2-trifluoro-1-methoxyethyl group, a 2,2,2-trifluoro-1-ethoxyethyl group, a 2,2,2-trifluoro-1-propoxyethyl group, a 2,2,2,3,3-pentafluoro-1-methoxypropyl group, a 2,2,2,3,3-pentafluoro-1-ethoxypropyl group, a 2,2,2-trifluoro-1-(2-propynyloxy)ethyl group, a 2,2,2-trifluoro-1-(2-propenyloxy)ethyl group, and a 2,2,2-trifluoro-1-methyl-1-(trimethylsilyloxy)ethyl group.

Examples of the “phenyl group optionally substituted with one or more members selected from Group β” represented by R¹, R⁷ or R⁸ include a phenyl group, a 2-chlorophenyl group, a 3-chlorophenyl group, a 4-chlorophenyl group, a 3-fluorophenyl group, a 4-fluorophenyl group, a 3-bromophenyl group, a 3-iodophenyl group, a 2-cyanophenyl group, a 3-cyanophenyl group, a 4-cyanophenyl group, a 2-nitrophenyl group, a 3-nitrophenyl group, a 4-nitrophenyl group, a 2-methylphenyl group, a 3-methylphenyl group, a 4-methylphenyl group, a 2-(trifluoromethyl)phenyl group, a 3-(trifluoromethyl)phenyl group, a 4-(trifluoromethyl)phenyl group, a 2-methoxyphenyl group, a 3-methoxyphenyl group, a 4-methoxyphenyl group, a 3-(trifluoromethoxy)phenyl group, a 4-(trifluoromethoxy)phenyl group, a 3-tert-butylphenyl group, a 2,4-dichlorophenyl group, a 2,4-difluorophenyl group, a 2,3-dichlorophenyl group, a 2,3-difluorophenyl group, a 3,4-dichlorophenyl group, a 3,4-difluorophenyl group, a 2,4,6-trifluorophenyl group, and a 2,4,6-trichlorophenyl group.

Examples of the “pyridyl group optionally substituted with one or more members selected from Group β” represented by R¹ include a 2-pyridyl group, a 3-pyridyl group, a 4-pyridyl group, a 3-chloro-(pyridin-2-yl) group, a 4-chloro-(pyridin-2-yl) group, a 5-chloro-(pyridin-2-yl) group, a 6-chloro-(pyridin-2-yl) group, a 3-fluoro-(pyridin-2-yl) group, a 4-fluoro-(pyridin-2-yl) group, a 5-fluoro-(pyridin-2-yl) group, a 6-fluoro-(pyridin-2-yl) group, a 3-methyl-(pyridin-2-yl) group, a 4-methyl-(pyridin-2-yl) group, a 5-methyl-(pyridin-2-yl) group, a 6-methyl-(pyridin-2-yl) group, a 3-trifluoromethyl-(pyridin-2-yl) group, a 4-trifluoromethyl-(pyridin-2-yl) group, a 5-trifluoromethyl-(pyridin-2-yl) group, a 6-trifluoromethyl-(pyridin-2-yl) group, a 3-methoxy-(pyridin-2-yl) group, a 4-methoxy-(pyridin-2-yl) group, a 5-methoxy-(pyridin-2-yl) group, a 6-methoxy-(pyridin-2-yl) group, a 3-trifluoromethoxy-(pyridin-2-yl) group, a 4-trifluoromethoxy-(pyridin-2-yl) group, a 5-trifluoromethoxy-(pyridin-2-yl) group, a 6-trifluoromethoxy-(pyridin-2-yl) group, a 3-pentafluoroethyl-(pyridin-2-yl) group, a 4-pentafluoroethyl-(pyridin-2-yl) group, a 5-pentafluoroethyl-(pyridin-2-yl) group, a 6-pentafluoroethyl-(pyridin-2-yl) group, a 3-heptafluoroisopropyl-(pyridin-2-yl) group, a 4-heptafluoroisopropyl-(pyridin-2-yl) group, a 5-heptafluoroisopropyl-(pyridin-2-yl) group, a 6-heptafluoroisopropyl-(pyridin-2-yl) group, a 2-chloro-(pyridin-3-yl) group, a 4-chloro-(pyridin-3-yl) group, a 5-chloro-(pyridin-3-yl) group, a 6-chloro-(pyridin-3-yl) group, a 2-fluoro-(pyridin-3-yl) group, a 4-fluoro-(pyridin-3-yl) group, a 5-fluoro-(pyridin-3-yl) group, a 6-fluoro-(pyridin-3-yl) group, a 2-trifluoromethyl-(pyridin-3-yl) group, a 4-trifluoromethyl-(pyridin-3-yl) group, a 5-trifluoromethyl-(pyridin-3-yl) group, a 6-trifluoromethyl-(pyridin-3-yl) group, a 2-pentafluoroethyl-(pyridin-3-yl) group, a 4-pentafluoroethyl-(pyridin-3-yl) group, a 5-pentafluoroethyl-(pyridin-3-yl) group, a 6-pentafluoroethyl-(pyridin-3-yl) group, a 2-trifluoromethoxy-(pyridin-3-yl) group, a 4-trifluoromethoxy-(pyridin-3-yl) group, a 5-trifluoromethoxy-(pyridin-3-yl) group, a 6-trifluoromethoxy-(pyridin-3-yl) group, a 2-chloro-(pyridin-4-yl) group, a 3-chloro-(pyridin-4-yl) group, a 2-fluoro-(pyridin-4-yl) group, a 3-fluoro-(pyridin-4-yl) group, a 2-trifluoromethyl-(pyridin-4-yl) group, a 3-trifluoromethyl-(pyridin-4-yl) group, a 2-trifluoromethoxy-(pyridin-4-yl) group, a 3-trifluoromethoxy-(pyridin-4-yl) group, a 2-pentafluoroethyl-(pyridin-4-yl) group, a 3-pentafluoroethyl-(pyridin-4-yl) group, a 3,6-bis(trifluoromethyl)-(pyridin-2-yl) group, a 2,4-bis(trifluoromethyl)-(pyridin-3-yl) group, and a 3,5-bis(trifluoromethyl)-(pyridin-4-yl) group.

Examples of the “C1-C7 alkyl group optionally substituted with halogen” represented by R², R³, R⁴, R⁵, R⁶, R⁷, R⁸, R⁹ or R¹⁰ include C1-C7 alkyl groups such as a methyl group, an ethyl group, a propyl group, a 2-methylpropyl group, a 1-methylpropyl group, a 1,1-dimethylethyl group, a 3-methylbutyl group, a 2,2-dimethylpropyl group, a 1,1-dimethylpropyl group, a hexyl group, a 4-methylpentyl group, a 3-methylpentyl group, a 1,3-dimethylbutyl group, a heptyl group, and a 1-ethyl-1-methylbutyl group; and
C1-C7 haloalkyl groups such as a fluoromethyl group, a chloromethyl group, a bromomethyl group, a difluoromethyl group, a dichloromethyl group, a dibromomethyl group, a trifluoromethyl group, a trichloromethyl group, a dichlorofluoromethyl group, a chlorodifluoromethyl group, a trifluoromethyl group, a difluoromethyl group, a 2,2,2-trifluoroethyl group, a 1,1,2,2,2-pentafluoroethyl group, a 3,3,3-trifluoropropyl group, a 2,2,3,3,3-pentafluoropropyl group, a heptafluoropropyl group, a 1-methyl-2,2,2-trifluoroethyl group, a 1-trifluoromethyl-2,2,2-trifluoroethyl group, and a heptafluoroisopropyl group.

Examples of the “C3-C7 alkenyl group optionally substituted with halogen” represented by R⁵, R⁶ or R¹⁰ include a 2-propenyl group, a 3-butenyl group, a 1-methyl-2-propenyl group, a 2-methyl-2-propenyl group, a 2-pentenyl group, a 1-methyl-2-butenyl group, a 3-methyl-3-butenyl group, a 1-ethyl-2-propenyl group, a 2-hexenyl group, a 2-methyl-2-pentenyl group, a 3-methyl-2-pentenyl group, a 4-methyl-2-pentenyl group, a 1-methyl-3-pentenyl group, a 4-methyl-3-pentenyl group, a 1-methyl-4-pentenyl group and 4-methyl-4-pentenyl group, a 3-chloro-2-propenyl group, a 3,3-dichloro-2-propenyl group, a 4,4-dichloro-3-butenyl group, and a 2-chloro-2-propenyl group.

Examples of the “C3-C7 alkynyl group optionally substituted with halogen” represented by R¹⁰ include C3-C7 alkynyl groups such as a 2-propynyl group, a 2-butynyl group, and a 3-butynyl group; and
C3-C7 haloalkynyl groups such as a 4-chloro-2-butynyl group, a 4,4,4-trifluoro-2-butynyl group, a 1-(trifluoromethyl)-2-butynyl group, and a 1-(trifluoromethyl)-2-propynyl group.

Examples of the “C2-C7 alkoxyalkyl group” represented by R¹⁰ include a methoxymethyl group, a 2-methoxyethyl group, and a 2-ethoxyethyl group.

Examples of the “(C3-C7 cycloalkyl)methyl group optionally substituted with one or more members selected from Group α” represented by R² include a (cyclopropyl)methyl group, a (1-methylcyclopropyl)methyl group, a (2,2-dimethylcyclopropyl)methyl group, a (cyclopentyl)methyl group, and cyclohexylmethyl group.

Examples of the “benzyl group optionally substituted with one or more members selected from Group β” represented by R² or R⁷ include a benzyl group, a 1-phenylethyl group, a 2-chlorobenzyl group, a 3-chlorobenzyl group, a 4-chlorobenzyl group, a 3-bromobenzyl group, a 4-bromobenzyl group, a 2-fluorobenzyl group, a 3-fluorobenzyl group, a 2-cyanobenzyl group, a 3-cyanobenzyl group, a 4-cyanobenzyl group, a 2-nitrobenzyl group, a 3-nitrobenzyl group, a 4-nitrobenzyl group, a 2-methylbenzyl group, a 3-methylbenzyl group, a 4-methylbenzyl group, a 2-(trifluoromethyl)benzyl group, a 3-(trifluoromethyl)benzyl group, a 4-(trifluoromethyl)benzyl group, a 2-methoxybenzyl group, a 3-methoxybenzyl group, and a 4-methoxybenzyl group.

Examples of the “C3-C7 cycloalkyl group optionally substituted with one or more members selected from Group α” represented by R³, R⁴, R⁵, R⁶, R⁷ or R⁸ include a cyclopropyl group, a 1-methylcyclopropyl group, a 2-methylcyclopropyl group, a 2,2-dimethylcyclopropyl group, a 2-fluorocyclopropyl group, a cyclobutyl group, a 1-trifluoromethylcyclobutyl group, a cyclopentyl group, a 2-methylcyclopentyl group, a cyclohexyl group, a 1-methylcyclohexyl group, a 2-methylcyclohexyl group, a 3-methylcyclohexyl group, a 4-methylcyclohexyl group, a 4-trifluoromethylcyclohexyl group, a 2-fluorohexyl group, a 3-fluorohexyl group, a 4-fluorocyclohexyl group, and a cycloheptyl group.

Examples of the “pyrrolidin-1-yl group optionally substituted with one or more members selected from Group α” represented by R⁵ or R⁶ include a pyrrolidin-1-yl group, a 2-methylpyrrolidin-1-yl group, and a 3,5-dimethylpyrrolidin-1-yl group.

Examples of the “piperidino group optionally substituted with one or more members selected from Group α” represented by R⁵ or R⁶ include a piperidino group, a 2-methylpiperidino group, a 3-methylpiperidino group, a 3,5-dimethylpiperidino group, and a 4-tert-butylpiperidino group.

Examples of the “hexamethyleneimin-1-yl group optionally substituted with one or more members selected from Group α” represented by R⁵ or R⁶ include a hexamethyleneimin-1-yl group.

Examples of the “morpholino group optionally substituted with one or more members selected from Group α” represented by R⁵ or R⁶ include a morpholino group and a 3,5-dimethylmorpholino group.

Examples of the “thiomorpholin-4-yl group optionally substituted with one or more members selected from Group α” represented by R⁵ or R⁶ include a thiomorpholin-4-yl group.

Examples of the “C1-C7 alkyloxy group optionally substituted with halogen” represented by R³ or R⁵ include C1-C7 alkyloxy groups such as a methoxy group, an ethoxy group, a propyloxy group, an isopropyloxy group, a hexyloxy group, a 4-methylpentyloxy group, a 3-methylpentyloxy group, a 1,3-dimethylbutyloxy group, a heptyloxy group, and a 1-ethyl-1-methylbutyloxy group; and
C1-C7 haloalkyloxy groups such as a fluoromethoxy group, a chloromethoxy group, a bromomethoxy group, a difluoromethoxy group, a dichloromethoxy group, a dibromomethoxy group, a trifluoromethoxy group, a trichloromethoxy group, a dichlorofluoromethoxy group, a chlorodifluoromethoxy group, a difluorobromomethoxy group, a 2,2,2-trifluoroethoxy group, a pentafluoroethoxy group, a 3,3,3,2,2-pentafluoropropyloxy group, a 1-methyl-2,2,2-trifluoroethoxy group, and a 1-trifluoromethyl-2,2,2-trifluoroethoxy group.

Examples of the “C1-C7 alkylthio group optionally substituted with halogen” represented by R³ include C1-C7 alkylthio groups such as a methylthio group, an ethylthio group, a propylthio group, a 2-methylpropylthio group, a 1-methylpropylthio group, a 1,1-dimethylethylthio group, a 3-methylbutylthio group, a 2,2-dimethylpropylthio group, a 1,1-dimethylpropylthio group, a hexylthio group, a 4-methylpentylthio group, a 3-methylpentylthio group, and a 1,3-dimethylbutylthio group; and
C1-C7 haloalkylthio groups such as a trifluoromethylthio group, a trichloromethylthio group, a difluoromethylthio group, a 2,2,2-trifluoroethylthio group, a 1,1,2,2,2-pentafluoroethylthio group, a 3,3,3-trifluoropropylthio group, a 2,2,3,3,3-pentafluoropropylthio group, a heptafluoropropylthio group, a 1-methyl-2,2,2-trifluoroethylthio group, a 1-trifluoromethyl-2,2,2-trifluoroethylthio group, and a heptafluoroisopropylthio group.

Examples of the “C3-C7 cycloalkyloxy group optionally substituted with one or more members selected from Group α” represented by R³ include a cyclopropyloxy group, a 1-methylcyclopropyloxy group, a 2-methylcyclopropyloxy group, a 2,2-dimethylcyclopropyloxy group, a 2-fluorocyclopropyloxy group, a cyclobutyloxy group, a 1-trifluoromethylcyclobutyloxy group, a cyclopentyloxy group, a 2-methyloyclopentyloxy group, a cyclohexyloxy group, a 1-methylcyclohexyloxy group, a 2-methylcyclohexyloxy group, a 3-methylcyclohexyloxy group, a 4-methylcyclohexyloxy group, a 4-trifluoromethylcyclohexyloxy group, a 2-fluorohexyloxy group, a 3-fluorohexyloxy group, a 4-fluorocyclohexyloxy group, and a cycloheptyloxy group.

Examples of the present compound include the following pyrimidine compounds such as:
a pyrimidine compound represented by formula (I), wherein R¹ is a C1-C3 haloalkyl group optionally substituted with one or more members selected from the group consisting of a C1-C3 alkyloxy group, a C3-C7 alkenyloxy group, a C3-C7 alkynyloxy group, a tri(C1-C4 alkyl)silyloxy group, and a hydroxy group, a phenyl group optionally substituted with one or more members selected from Group α, or a pyridyl group optionally substituted with one or more members selected from Group α;
a pyrimidine compound represented by formula (I), wherein R¹ is a C1-C3 haloalkyl group optionally substituted with one or more C1-C3 alkyloxy groups;
a pyrimidine compound represented by formula (I), wherein R¹ is halogen, a phenyl group optionally substituted with one or more C1-C3 haloalkyl groups, or a pyridyl group optionally substituted with one or more C1-C3 haloalkyl groups;
a pyrimidine compound represented by formula (I), wherein R¹ is a C1-C3 fluoroalkyl group optionally substituted with one or more C1-C3 alkyloxy groups;
a pyrimidine compound represented by formula (I), wherein R¹ is halogen, a phenyl group optionally substituted with one or more C1-C3 fluoroalkyl groups, or a pyridyl group optionally substituted with halogen or one or more C1-C3 fluoroalkyl groups;
a pyrimidine compound represented by formula (I), wherein n is 0;
a pyrimidine compound represented by formula (I), wherein R² is hydrogen
a pyrimidine compound represented by formula (I), wherein R² is any one of Q¹ to Q⁵;
a pyrimidine compound represented by formula (I), wherein R² is Q¹;
a pyrimidine compound represented by formula (I), wherein R² is Q²;
a pyrimidine compound represented by formula (I), wherein R² is Q³;
a pyrimidine compound represented by formula (I), wherein R² is Q⁴;
a pyrimidine compound represented by formula (I), wherein R² is Q⁵;
a pyrimidine compound represented by formula (I), wherein R¹ is a C1-C7 haloalkyl group optionally substituted with one or more members selected from Group γ;
a pyrimidine compound represented by formula (I), wherein R¹ is a phenyl group optionally substituted with one or more members selected from Group β, or a pyridyl group optionally substituted with one or more members selected from Group β;
a pyrimidine compound represented by formula (I), wherein R⁴ is a C1-C7 alkyl group, or a C3-C7 cycloalkyl group optionally substituted with one or more C1-C3 alkyl groups;
a pyrimidine compound represented by formula (I), wherein —NR⁵R⁶ is replaced by a pyrrolidin-1-yl group optionally substituted with one or more C1-C3 alkyl groups, a piperidino group optionally substituted with one or more C1-C3 alkyl groups, a hexamethyleneimin-1-yl group optionally substituted with one or more C1-C3 alkyl groups, a morpholino group optionally substituted with one or more C1-C3 alkyl groups, or a thiomorpholin-4-yl group optionally substituted with one or more C1-C3 alkyl groups;
a pyrimidine compound represented by formula (I), wherein R⁵ and R⁶ are the same or different and represent a C1-C7 alkyl group optionally substituted with halogen;
a pyrimidine compound represented by formula (I), wherein R⁷ is a phenyl group optionally substituted with one or more members selected from Group α, or a benzyl group optionally substituted with one or more members selected from Group α;
a pyrimidine compound represented by formula (I), wherein R⁸ is a C1-C7 alkyl group, a phenyl group optionally substituted with one or more members selected from Group α, or a C3-C7 cycloalkyl group;
a pyrimidine compound represented by formula (I), wherein R³ is a C1-C3 alkyl group optionally substituted with halogen, a C1-C3 alkylthio group optionally substituted with halogen, a C1-C3 alkyloxy group optionally substituted with halogen, a C3-C7 cycloalkyl group optionally substituted with one or more C1-C3 alkyl groups, or a C3-C7 cycloalkyloxy group optionally substituted with one or more C1-C3 alkyl groups;
a pyrimidine compound represented by formula (I), wherein R³ is a C1-C3 alkyl group optionally substituted with halogen;
a pyrimidine compound represented by formula (I), wherein R³ is a C1-C3 alkyloxy group optionally substituted with halogen;
a pyrimidine compound represented by formula (I), wherein R³ is a C3-C7 cycloalkyl group optionally substituted with one or more C1-C3 alkyl groups;
a pyrimidine compound represented by formula (I), wherein R³ is a C3-C7 cycloalkyloxy group optionally substituted with one or more C1-C3 alkyl groups;
a pyrimidine compound represented by formula (I), wherein R¹ is a C1-C3 haloalkyl group optionally substituted with one or more members selected from the group consisting of a C1-C3 alkyloxy group, a C3-C7 alkenyloxy group, a C3-C7 alkynyloxy group, a tri(C1-C4 alkyl)silyloxy group, and a hydroxy group,
A is a single bond, —CH₂—, or oxygen,
R² is hydrogen or any one of Q1 to Q5,
R⁴ is a C1-C7 alkyl group, or a C3-C7 cycloalkyl group optionally substituted with one or more C1-C3 alkyl groups,
R⁵ and R⁶ are the same or different and represent a C1-C7 alkyl group optionally substituted with halogen, or —NR⁵R⁶ is replaced by a pyrrolidin-1-yl group optionally substituted with one or more C1-C3 alkyl groups, a piperidino group optionally substituted with one or more C1-C3 alkyl groups, a hexamethyleneimin-1-yl group optionally substituted with one or more C1-C3 alkyl groups, a morpholino group optionally substituted with one or more C1-C3 alkyl groups, or a thiomorpholin-4-yl group optionally substituted with one or more C1-C3 alkyl groups,
R⁷ is a phenyl group optionally substituted with one or more members selected from Group α, or a benzyl group optionally substituted with one or more members selected from Group α,
R⁸ is a C1-C7 alkyl group, a phenyl group optionally substituted with one or more members selected from Group α,
or a C3-C7 cycloalkyl group, and
R³ is a C1-C3 alkyl group optionally substituted with halogen, a C1-C3 alkylthio group optionally substituted with halogen, a C1-C3 alkyloxy group optionally substituted with halogen, a C3-C7 cycloalkyl group optionally substituted with one or more C1-C3 alkyl groups, or a C3-C7 cycloalkyloxy group optionally substituted with one or more C1-C3 alkyl groups;
a pyrimidine compound represented by formula (I), wherein R¹ is a C1-C3 haloalkyl group, and R² is hydrogen;
a pyrimidine compound represented by formula (I), wherein R¹ is a C1-C3 haloalkyl group, and R² is Q¹;
a pyrimidine compound represented by formula (I), wherein R¹ is a C1-C3 haloalkyl group, and R² is Q²;
a pyrimidine compound represented by formula (I), wherein R¹ is a C1-C3 haloalkyl group, and R² is Q³;
a pyrimidine compound represented by formula (I), wherein R¹ is a C1-C3 haloalkyl group, and R² is Q⁴;
a pyrimidine compound represented by formula (I), wherein R¹ is a C1-C3 haloalkyl group, and R² is Q⁵;
a pyrimidine compound represented by formula (I), wherein R¹ is a C1-C3 haloalkyl group, and R³ is hydrogen;
a pyrimidine compound represented by formula (I), wherein R¹ is a C1-C3 haloalkyl group, and R³ is a C3-C7 cycloalkyl group;
a pyrimidine compound represented by formula (I), wherein R¹ is a phenyl group optionally substituted with one or more members selected from Group α, and R² is hydrogen;
a pyrimidine compound represented by formula (I), wherein R¹ is a phenyl group optionally substituted with one or more members selected from Group α, and R² is Q¹;
a pyrimidine compound represented by formula (I), wherein R¹ is a phenyl group optionally substituted with one or more members selected from Group α, and R² is Q²;
a pyrimidine compound represented by formula (I), wherein R¹ is a phenyl group optionally substituted with one or more members selected from Group α, and R² is Q³;
a pyrimidine compound represented by formula (I), wherein R¹ is a phenyl group optionally substituted with one or more members selected from Group α, and R² is Q⁴;
a pyrimidine compound represented by formula (I), wherein R¹ is a phenyl group optionally substituted with one or more members selected from Group α, and R² is Q⁵;
a pyrimidine compound represented by formula (I), wherein R¹ is a phenyl group optionally substituted with one or more members selected from Group α, and R³ is hydrogen;
a pyrimidine compound represented by formula (I), wherein R¹ is a phenyl group optionally substituted with one or more members selected from Group α, and R³ is a C3-C7 cycloalkyl group;
a pyrimidine compound represented by formula (I), wherein R¹ is a C1-C3 haloalkyl group, A is a single bond, oxygen, or —CH₂—, and R² is hydrogen;
a pyrimidine compound represented by formula (I), wherein R¹ is a C1-C3 haloalkyl group, A is a single bond, oxygen, or —CH₂—, and R² is Q¹;
a pyrimidine compound represented by formula (I), wherein R¹ is a C1-C3 haloalkyl group, A is a single bond, oxygen, or —CH₂—, and R² is Q²;
a pyrimidine compound represented by formula (I), wherein R¹ is a C1-C3 haloalkyl group, A is a single bond, oxygen, or —CH₂—, and R² is Q³;
a pyrimidine compound represented by formula (I), wherein R¹ is a C1-C3 haloalkyl group, A is a single bond, oxygen, or —CH₂—, and R² is Q⁴;
a pyrimidine compound represented by formula (I), wherein R¹ is a C1-C3 haloalkyl group, A is a single bond, oxygen, or —CH₂—, and R² is Q⁵;
a pyrimidine compound represented by formula (I), wherein R¹ is a C1-C3 haloalkyl group, A is a single bond, oxygen, or —CH₂—, and R³ is hydrogen;
a pyrimidine compound represented by formula (I), wherein R¹ is a C1-C3 haloalkyl group, A is a single bond, oxygen, or —CH₂—, and R³ is a C3-C7 cycloalkyl group;
a pyrimidine compound represented by formula (I), wherein R¹ is a phenyl group optionally substituted with one or more members selected from Group α, A is a single bond, oxygen, or —CH₂—, and R² is hydrogen;
a pyrimidine compound represented by formula (I), wherein R¹ is a phenyl group optionally substituted with one or more members selected from Group α, A is a single bond, oxygen, or —CH₂—, and R² is Q¹;
a pyrimidine compound represented by formula (I), wherein R¹ is a phenyl group optionally substituted with one or more members selected from Group α, A is a single bond, oxygen, or —CH₂—, and R² is Q²;
a pyrimidine compound represented by formula (I), wherein R¹ is a phenyl group optionally substituted with one or more members selected from Group α, A is a single bond, oxygen, or —CH₂—, and R² is Q³;
a pyrimidine compound represented by formula (I), wherein R¹ is a phenyl group optionally substituted with one or more members selected from Group α, A is a single bond, oxygen, or —CH₂—, and R² is Q⁴;
a pyrimidine compound represented by formula (I), wherein R¹ is a phenyl group optionally substituted with one or more members selected from Group α, A is a single bond, oxygen, or —CH₂—, and R² is Q⁵;
a pyrimidine compound represented by formula (I), wherein R¹ is a phenyl group optionally substituted with one or more members selected from Group α, A is a single bond, oxygen, or —CH₂—, and R³ is hydrogen;
a pyrimidine compound represented by formula (I), wherein R¹ is a phenyl group optionally substituted with one or more members selected from Group α, A is a single bond, oxygen, or —CH₂—, and R³ is a C3-C7 cycloalkyl group;
a pyrimidine compound represented by formula (I), wherein R¹ is a pyridyl group optionally substituted with one or more members selected from Group α, and R² is hydrogen;
a pyrimidine compound represented by formula (I), wherein R¹ is a pyridyl group optionally substituted with one or more members selected from Group α, and R² is Q¹;
a pyrimidine compound represented by formula (I), wherein R¹ is a pyridyl group optionally substituted with one or more members selected from Group α, and R² is Q²;
a pyrimidine compound represented by formula (I), wherein R¹ is a pyridyl group optionally substituted with one or more members selected from Group α, and R² is Q³;
a pyrimidine compound represented by formula (I), wherein R¹ is a pyridyl group optionally substituted with one or more members selected from Group α, and R² is Q⁴;
a pyrimidine compound represented by formula (I), wherein R¹ is a pyridyl group optionally substituted with one or more members selected from Group α, and R² is Q⁵;
a pyrimidine compound represented by formula (I), wherein R¹ is a pyridyl group optionally substituted with one or more members selected from Group α, and R³ is hydrogen;
a pyrimidine compound represented by formula (I), wherein R¹ is a pyridyl group optionally substituted with one or more members selected from Group α, and R³ is a C3-C7 cycloalkyl group;
a pyrimidine compound represented by formula (I), wherein R¹ is a pyridyl group optionally substituted with one or more members selected from Group α, A is a single bond or —CH₂—, and R² is hydrogen;
a pyrimidine compound represented by formula (I), wherein R¹ is a pyridyl group optionally substituted with one or more members selected from Group α, A is a single bond or —CH₂—, and R² is Q¹;
a pyrimidine compound represented by formula (I), wherein R¹ is a pyridyl group optionally substituted with one or more members selected from Group α, A is a single bond or —CH₂—, and R² is Q²;
a pyrimidine compound represented by formula (I), wherein R¹ is a pyridyl group optionally substituted with one or more members selected from Group α, A is a single bond or —CH₂—, and R² is Q³;
a pyrimidine compound represented by formula (I), wherein R¹ is a pyridyl group optionally substituted with one or more members selected from Group α, A is a single bond or —CH₂—, and R² is Q⁴;
a pyrimidine compound represented by formula (I), wherein R¹ is a pyridyl group optionally substituted with one or more members selected from Group α, A is a single bond or —CH₂—, and R² is Q⁵;
a pyrimidine compound represented by formula (I), wherein R¹ is a pyridyl group optionally substituted with one or more members selected from Group α, A is a single bond or —CH₂—, and R³ is hydrogen; and
a pyrimidine compound represented by formula (I), wherein R¹ is a pyridyl group optionally substituted with one or more members selected from Group α, A is a single bond or —CH₂—, and R³ is a C3-C7 cycloalkyl group.

Processes for producing the present compound will be described below.

The present compound can be produced, for example, by the following Production Processes 1 to 4.

### (Production Process 1)

Among the present compound, the compound represented by formula (1-0) can be produced, for example, from the compound represented by formula (1-1) by the following process:

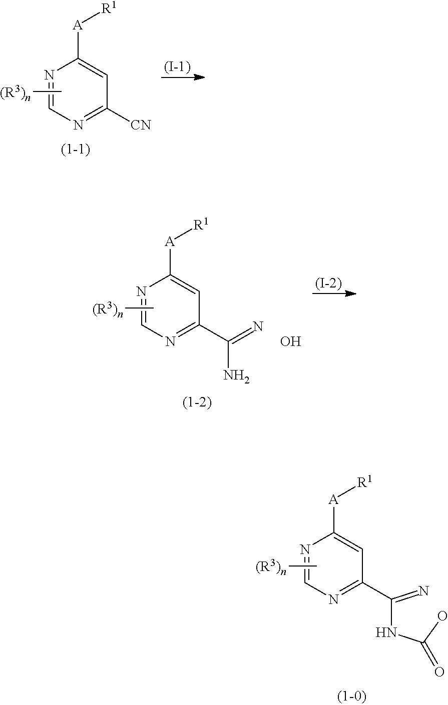

wherein A, R¹, R³ and n are as defined above.

### Step (I-1)

The compound represented by formula (1-2) can be produced by reacting the compound represented by formula (1-1) with hydroxylamine in the presence of a base.

The reaction is usually conducted in a solvent. Examples of the solvent used in the reaction include alcohols such as methanol, ethanol and 2-propanol; water; and mixtures thereof.

Examples of the base used in the reaction include metal hydrides such as sodium hydride; and carbonates such as sodium hydrogen carbonate and potassium carbonate. The amount of the base used in the reaction is usually from 1 to 4 mol based on 1 mol of the compound represented by formula (1-1).

Examples of the hydroxylamine used in the reaction include hydroxylamine, hydroxylamine hydrochloride, and hydroxylamine sulfate. The amount of the hydroxylamine used in the reaction is usually from 1 to 3 mol based on 1 mol of the compound represented by formula (1-1). The reaction temperature of the reaction is usually within a range from 0 to 120° C. The reaction time of the reaction is usually within a range from 0.1 to 46 hours.

After completion of the reaction, the reaction mixture is subjected to a post-treatment such as concentration or extraction with an organic solvent, and thus the compound represented by formula (1-2) can be isolated. The isolated compound represented by formula (1-2) can also be further purified by recrystallization, chromatography or the like.

### Step (I-2)

The compound represented by formula (1-0) can be produced by reacting the compound represented by formula (1-2) with a carbonylating agent in the presence of a base.

The reaction is usually conducted in a solvent. Examples of the solvent used in the reaction include ethers such as 1,4-dioxane, diethylether, tetrahydrofuran, and tert-butyl methyl ether; halogenated hydrocarbons such as dichloromethane, chloroform, carbon tetrachloride, 1,2-dichloroethane, and chlorobenzene; hydrocarbons such as toluene, benzene, and xylene; nitriles such as acetonitrile; aprotic polar solvents such as N,N-dimethylformamide, N-methyl pyrrolidone, and dimethyl sulfoxide; and mixtures thereof.

Examples of the base used in the reaction include nitrogen-containing heterocyclic compounds such as pyridine, picoline, 2,6-lutidine, and 1,8-diazabicyclo[5.4.0]undec-7-ene; and tertiary amines such as triethylamine and N,N-diisopropylethylamine. The amount of the base used in the reaction is usually 1 to 3 mol based on 1 mol of the compound represented by formula (1-2).

Examples of the carbonylating agent used in the reaction include phosgene and 1,1′-carbonyldiimidazole. The amount of the carbonylating agent used in the reaction is usually from 1 to 3 mol based on 1 mol of the compound represented by formula (1-2).

The reaction temperature of the reaction is usually within a range from 0 to 100° C. The reaction time of the reaction is usually within a range from 0.1 to 48 hours.

After completion of the reaction, the reaction mixture is subjected to a post-treatment such as concentration or extraction with an organic solvent, and thus the compound represented by formula (1-0) can be isolated. The isolated compound represented by formula (1-0) can also be further purified by recrystallization, chromatography or the like.

### (Production Process 2)

Among the present compound, the compound represented by formula (2) can be produced from the compound represented by formula (1-0) via step (II):

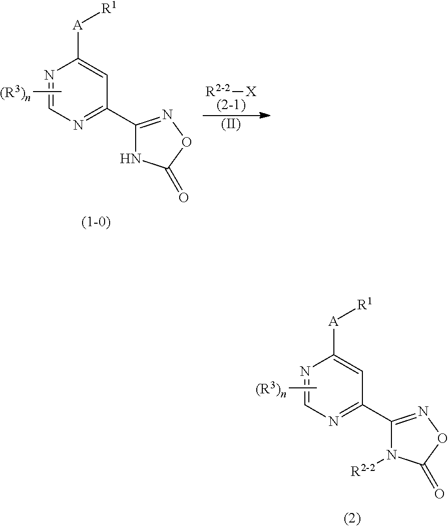

wherein A, R¹, R³ and n are as defined above, X represents a leaving group such as chlorine, bromine, iodine, a paratoluenesulfonyloxy group, or a methanesulfonyloxy group, and R²⁻² represents a group other than hydrogen among groups represented by R².

In step (II), the compound represented by formula (1-0) is reacted with the compound represented by formula (2-1) in the presence of a base.

The reaction is usually conducted in a solvent. Examples of the solvent used in the reaction include ethers such as 1,4-dioxane, diethylether, tetrahydrofuran, and tert-butyl methyl ether; hydrocarbons such as toluene, benzene, and xylene; nitriles such as acetonitrile; aprotic polar solvents such as N,N-dimethylformamide, N-methyl pyrrolidone, and dimethyl sulfoxide; pyridine, picoline, 2,6-lutidine, and mixtures thereof.

The base used in the reaction can be appropriately selected according to the solvent used in the reaction. Examples of the base used in the reaction include metal hydrides such as sodium hydride; carbonates such as potassium carbonate; nitrogen-containing heterocyclic compounds such as 1,8-diazabicyclo[5.4.0]undec-7-ene and 1,5-diazabicyclo[4.3.0]non-5-ene; and tertiary amines such as triethylamine and N,N-diisopropylethylamine. The amount of the base used in the reaction is usually 1 to 3 mol based on 1 mol of the compound represented by formula (1-0). The compound represented by formula (2-1) used in the reaction is usually from 1 to 3 mol based on 1 mol of the compound represented by formula (1-0). The reaction temperature of the reaction is usually within a range from 0 to 120° C. The reaction time of the reaction is usually within a range from 0.1 to 36 hours.

After completion of the reaction, the reaction mixture is subjected to a post-treatment such as concentration or extraction with an organic solvent, and thus the compound represented by formula (2) can be isolated. The isolated compound represented by formula (2) can also be further purified by recrystallization, chromatography or the like.

### (Production Process 3)

Among the present compound, the compound represented by formula (3) can be produced from the compound represented by formula (1-0) via step (III):

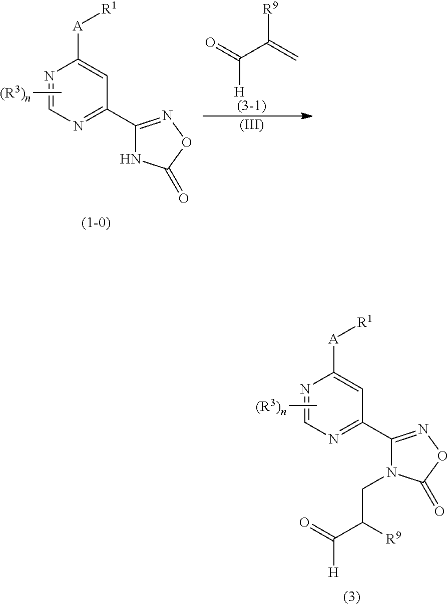

wherein A, R¹, R³, R⁹ and n are as defined above.

In step (III), the compound represented by formula (1-0) is reacted with the compound represented by formula (3-1) in the presence of a base.

The reaction is usually conducted in a solvent. Examples of the solvent used in the reaction include alcohols such as methanol and ethanol; halogenated hydrocarbons such as chloroform and dichloromethane; and mixtures thereof.

Examples of the base used in the reaction include nitrogen-containing heterocyclic compounds such as pyridine, picoline, 2,6-lutidine, 1,8-diazabicyclo[5.4.0]undec-7-ene, and 1,5-diazabicyclo[4.3.0]non-5-ene; and tertiary amines such as triethylamine and N,N-diisopropylethylamine. The amount of the base used in the reaction is usually 1 to 3 mol based on 1 mol of the compound represented by formula (1-0).

The amount of the compound represented by formula (3-1) used in the reaction is usually from 1 to 3 mol based on 1 mol of the compound represented by formula (1-0).

The reaction temperature of the reaction is usually within a range from 0 to 100° C. The reaction time of the reaction is usually within a range froth 0.1 to 48 hours.

After completion of the reaction, the reaction mixture is subjected to a post-treatment such as concentration or extraction with an organic solvent, and thus the compound represented by formula (3) can be isolated. The isolated compound represented by formula (3) can also be further purified by recrystallization, chromatography or the like.

### (Production Process 4)

Among the present compound, the compound represented by formula (5) can be produced from the compound represented by formula (1-0) via step (IV):

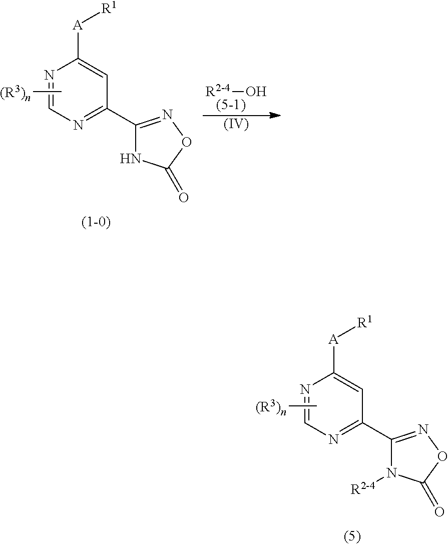

wherein A, R¹, R³ and n are as defined above, R²⁻⁴ is a C1-C7 alkyl group optionally substituted with halogen, a (C3-C7 cycloalkyl)methyl group optionally substituted with one or more members selected from Group α, a benzyl group optionally substituted with one or more members selected from Group β, or
any one of groups of the following Q³ and Q⁴ shown below:

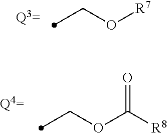

(wherein R⁷ represents a C1-C7 alkyl group optionally substituted with halogen, a phenyl group optionally substituted with one or more members selected from Group β, a benzyl group optionally substituted with one or more members selected from Group β, or a C3-C7 cycloalkyl group optionally substituted with one or more members selected from Group α, and
R⁸ represents a C1-C7 alkyl group optionally substituted with halogen, a phenyl group optionally substituted with one or more members selected from Group β, or a C3-C7 cycloalkyl group optionally substituted with one or more members selected from Group α).

In step (IV), the compound represented by formula (1-0) is reacted with the compound represented by formula (5-1) in the presence of triphenylphosphine and azodicarboxylic acids.

The reaction is usually conducted in a solvent. Examples of the solvent used in the reaction include ethers such as 1,4-dioxane, diethylether, tetrahydrofuran, and tert-butyl methyl ether; hydrocarbons such as toluene, benzene, and xylene; nitriles such as acetonitrile; aprotic polar solvents such as N,N-dimethylformamide, N-methyl pyrrolidone, and dimethyl sulfoxide; and mixtures thereof.

The amount of the triphenylphosphine used in the reaction is usually from 1 to 3 mol based on 1 mol of the compound represented by formula (1-0).

Examples of azodicarboxylic acids used in the reaction include an azodicarboxylic acid diethyl ester, an azodicarboxylic acid dibenzyl ester, 1,1′-azobis(N,N-dimethylformamide), and 1,1′-(azodicarbonyl)dipiperidine. The amount of azodicarboxylic acids used in the reaction is usually from 1 to 3 mol based on 1 mol of the compound represented by formula (1-0).

The amount of the compound represented by formula (5-1) used in the reaction is usually from 1 to 3 mol based on 1 mol of the compound represented by formula (1-0).

The reaction temperature of the reaction is usually within a range from 0 to 120° C. The reaction time of the reaction is usually within a range from 0.1 to 72 hours.

After completion of the reaction, the reaction mixture is subjected to a post-treatment such as concentration or extraction with an organic solvent, and thus the compound represented by formula (5) can be isolated. The isolated compound represented by formula (5) can also be further purified by recrystallization, chromatography or the like.

Processes for producing intermediates in the production of the present compound will be described below.

### Reference Production Process A

The compound represented by formula (1-1) can be produced from the compound represented by formula (a-1) via step (A-1) or (A-2):

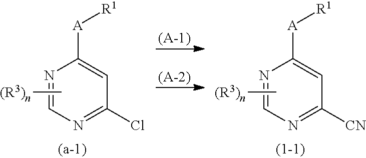

wherein A, R¹, R³ and n are as defined above.

### Step (A-1)

The compound represented by formula (1-1) can be produced by reacting the compound represented by formula (a-1) with zinc cyanide in the presence of a transition metal compound.

The reaction is usually conducted in a solvent. Examples of the solvent used in the reaction include ethers such as 1,4-dioxane, diethylether, tetrahydrofuran, and tert-butyl methyl ether; hydrocarbons such as toluene, benzene, and xylene; nitriles such as acetonitrile; aprotic polar solvents such as N,N-dimethylformamide, N-methyl pyrrolidone, and dimethyl sulfoxide; and mixtures thereof.

Examples of the transition metal compound used in the reaction include palladium compounds such as palladium acetate, tetrakis(triphenylphosphine)palladium, {1,1′-bis(diphenylphosphino)ferrocene}dichloropalladium(II) methylene chloride complex, and dichlorobis(triphenylphosphine)palladium(II). The amount of the transition metal compound used in the reaction can vary within a range where the object is achieved, and is usually from 0.01 to 0.1 mol based on 1 mol of the compound represented by formula (a-1).

The amount of zinc cyanide used in the reaction is usually from 0.5 to 2 mol based on 1 mol of the compound represented by formula (a-1).

The reaction temperature of the reaction is usually within a range from 0 to 150° C. The reaction time of the reaction is usually within a range from 0.1 to 72 hours.

After completion of the reaction, the reaction mixture is subjected to a post-treatment such as concentration or extraction with an organic solvent, and thus the compound represented by formula (1-1) can be isolated. The isolated compound represented by formula (1-1) can also be further purified by chromatography or the like.

### Step (A-2)

The compound represented by formula (1-1) can be produced from the compound represented by formula (a-1) using sodium cyanide in accordance with the method described in Japanese Unexamined Patent Publication (Kokai) No. 10-139765.

### Reference Production Process B

The compound represented by formula (b-2) can be produced from the compound represented by formula (b-1) via step (B):

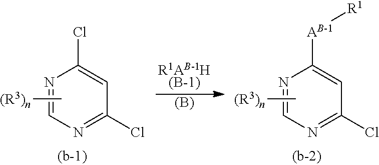

wherein R¹, R³, R¹⁰ and n are as defined above, and A^{B-1} represents oxygen, sulfur, —N(R¹⁰)—, or —CH₂O—.

### Step (B)

The compound represented by formula (b-1) is reacted with the compound represented by formula (B-1) in the presence of a base.

The reaction is usually conducted in a solvent. Examples of the solvent used in the reaction include ethers such as 1,4-dioxane, diethylether, tetrahydrofuran, and tert-butyl methyl ether; hydrocarbons such as toluene, benzene, and xylene; nitriles such as acetonitrile; aprotic polar solvents such as N,N-dimethylformamide, N-methyl pyrrolidone, and dimethyl sulfoxide; nitrogen-containing heterocyclic compounds such as pyridine, picoline, and 2,6-lutidine; and mixtures thereof.

The base used in the reaction can be appropriately selected according to the solvent used in the reaction. Examples of the base used in the reaction include metal hydrides such as sodium hydride; carbonates such as potassium carbonate; nitrogen-containing heterocyclic compounds such as 1,8-diazabicyclo[5.4.0]undeo-7-ene, and 1,5-diazabicyclo[4.3.0]non-5-ene; and tertiary amines such as triethylamine and N,N-diisopropylethylamine. The amount of the base used in the reaction is usually from 1 to 3 mol based on 1 mol of the compound represented by formula (b-1). The amount of the compound represented by formula (B-1) used in the reaction is usually from 1 to 3 mol based on 1 mol of the compound represented by formula (b-1). The reaction temperature of the reaction is usually within a range from 0 to 120° C. The reaction time of the reaction is usually within a range from 0.1 to 72 hours.

After completion of the reaction, the reaction mixture is subjected to a post-treatment such as concentration or extraction with an organic solvent, and thus the compound represented by formula (b-2) can be isolated. The isolated compound represented by formula (b-2) can also be further purified by recrystallization, chromatography or the like.

### Reference Production Process C

The compound represented by formula (c-2) can be produced from the compound represented by formula (b-1) via step (C):

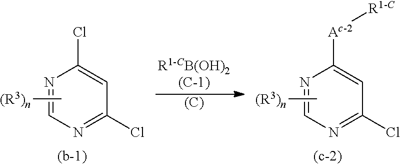

wherein R³ and n are as defined above, A^{c-2} represents a single bond, and R^{1-C} represents a phenyl group optionally substituted with one or more members selected from Group β, or a pyridyl group optionally substituted with one or more members selected from Group β.

### Step (C)

The compound represented by formula (c-2) can be produced by reacting the compound represented by formula (b-1) with a boronic acid compound represented by formula (C-1) under the atmosphere of an inert gas such as nitrogen or argon in the presence of a transition metal compound.

Examples of the transition metal compound used in the reaction include palladium compounds such as palladium acetate, tetrakis(triphenylphosphine)palladium, {1,1′-bis(diphenylphosphino)ferrocene}dichloropalladium(II) methylene chloride complex, and dichlorobis(triphenylphosphine)palladium(II). The amount of the transition metal compound used in the reaction can vary within a range where the object is achieved, and is usually from 0.001 to 0.1 mol based on 1 mol of the compound represented by formula (b-1).

The amount of a boronic acid compound represented by R^{1-c}B(OH)₂ used in the reaction is from 0.9 to 2.5 mol based on 1 mol of the compound represented by formula (b-1).

The reaction is usually conducted in a solvent. Examples of the solvent used in the reaction include alcohols such as methanol, ethanol, and 2-propanol; ethers such as 1,4-dioxane, tetrahydrofuran, 1,2-dimethoxyethane, and methyl-t-butyl ether; aliphatic hydrocarbons such as n-hexane and n-heptane; N,N-dimethylformamide, dimethyl sulfoxide, water, and mixtures thereof.

The reaction temperature of the reaction is usually from 0 to 150° C. The reaction time of the reaction is usually within a range from 0.1 to 96 hours.

The reaction can also be conducted optionally in the presence of a base and a phase transfer catalyst. Examples of the base used in the reaction include sodium acetate, potassium acetate, barium hydroxide, potassium carbonate, tripotassium phosphate, and sodium hydrogen carbonate. Examples of the phase transfer catalyst used in the reaction include quaternary ammonium salts such as tetrabutylammonium bromide and benzyltrimethylammonium bromide.

After completion of the reaction, the reaction mixture is subjected to a post-treatment such as concentration or extraction with an organic solvent, and thus the compound represented by formula (c-2) can be isolated. The isolated compound represented by formula (c-2) can also be purified by an operation such as chromatography.

### Reference Production Process D

The compound represented by formula (d-4) can be produced from the ester compound represented by formula (d-1) by the following process:

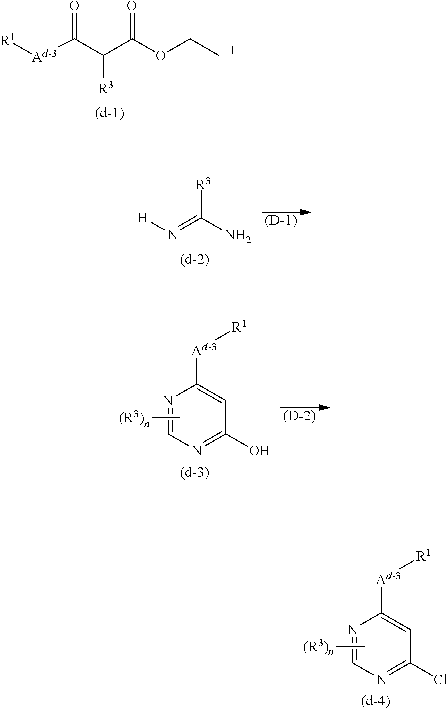

wherein R¹, R³ and n are as defined above, and A^{d-3} represents a single bond or —CH₂—.

### Step (D-1)

The compound represented by formula (d-3) can be produced by reacting the compound represented by formula (d-1) with an amidine compound represented by formula (d-2) in the presence of a base.

The reaction is usually conducted in a solvent. Examples of the solvent used in the reaction include alcohols such as methanol, ethanol, and 2-propanol; ethers such as 1,4-dioxane, diethylether, tetrahydrofuran, and tert-butyl methyl ether; hydrocarbons such as toluene, benzene, and xylene; aprotic polar solvents such as N,N-dimethylformamide, N-methyl pyrrolidone, and dimethyl sulfoxide; water, and mixtures thereof.

Examples of the base which can be used in the reaction include carbonates such as potassium carbonate; metal hydrides such as sodium hydride; sodium methoxide and sodium ethoxide. The amount of the base used in the reaction is usually from 1 to 10 mol based on 1 mol of the compound represented by formula (d-1). The amount of the amidine compound represented by formula (d-2) is from 1 to 4 mol based on 1 mol of the compound represented by formula (d-1).

The reaction temperature of the reaction is usually from 0 to 150° C. The reaction time of the reaction is usually within a range from 0.1 to 96 hours.

After completion of the reaction, the reaction mixture is extracted with an organic solvent and then subjected to a post-treatment such as concentration, extraction with an organic solvent, or collection a solid precipitated by adding an acid to the reaction mixture, and thus the compound represented by formula (d-3) can be isolated. The isolated compound represented by formula (d-3) can also be further purified by an operation such as recrystallization or chromatography.

### Step (D-2)

The compound represented by formula (d-4) can be produced by reacting the compound represented by formula (d-3) with thionyl chloride or phosphorus oxychloride.

The reaction is usually conducted in a solvent. Examples of the solvent used in the reaction include hydrocarbons such as toluene, benzene, and xylene; esters such as ethyl acetate; and mixtures thereof.

The amount of the thionyl chloride or phosphorus oxychloride used in the reaction is usually from 2 to 4 mol based on 1 mol of the compound represented by formula (d-3).

The reaction temperature of the reaction is usually from 0 to 150° C. The reaction time of the reaction is usually within a range from 0.1 to 48 hours.

After completion of the reaction, the reaction mixture is subjected to a post-treatment such as concentration or extraction with an organic solvent, and thus the compound represented by formula (d-4) can be obtained. The isolated compound represented by formula (d-4) can also be further purified by an operation such as chromatography.

### Reference Production Process E

The compound represented by formula (e-2) can be produced from the compound represented by formula (b-1) via step (E):

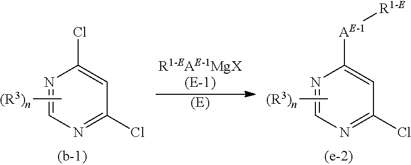

wherein R³ and n are as defined above, A^{E-1} represents a single bond or —CH₂—, X represents halogen, and R^{1-E} represents a phenyl group optionally substituted with one or more members selected from Group β.

Specific examples of the present compound are shown below. In the following compounds, Ph represents a phenyl group, 2-py represents a 2-pyridyl group, 3-py represents a 3-pyridyl group, and 4-py represents a 4-pyridyl group.

Compound represented by formula (4-1) (in which A, R¹ and R³ represent any one of combinations shown below).

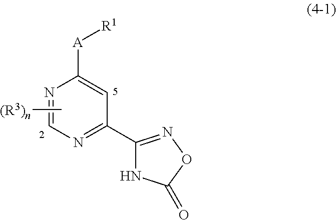

Compound represented by formula (4-2) (in which A, R¹ and R³ represent any one of combinations shown below).

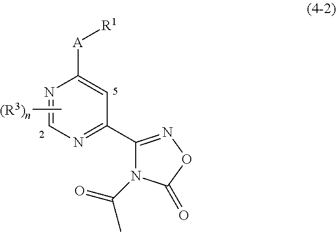

Compound represented by formula (4-3) (in which A, R¹ and R³ represent any one of combinations shown below).

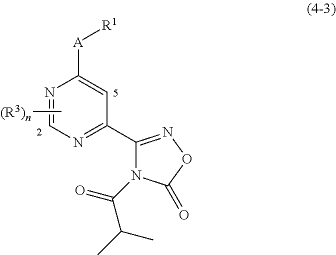

Compound represented by formula (4-4) (in which A, R¹ and R³ represent any one of combinations shown below).

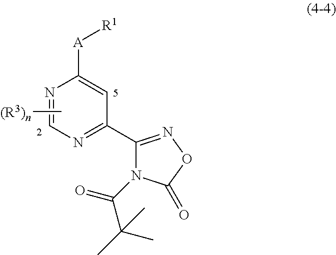

Compound represented by formula (4-5) (in which A, R¹ and R³ represent any one of combinations shown below).

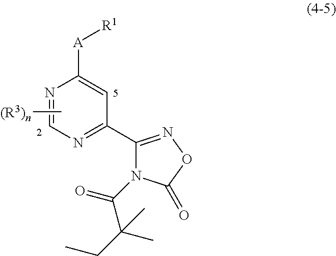

Compound represented by formula (4-6) (in which A, R¹ and R³ represent any one of combinations shown below).

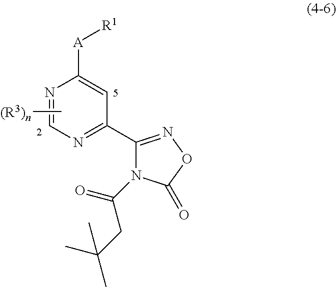

Compound represented by formula (4-7) (in which A, R¹ and R³ represent any one of combinations shown below).

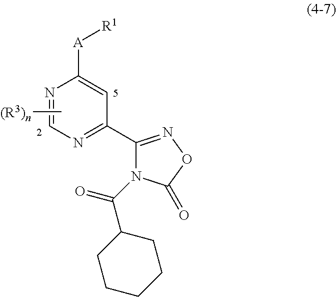

Compound represented by formula (4-8) (in which A, R¹ and R³ represent any one of combinations shown below).

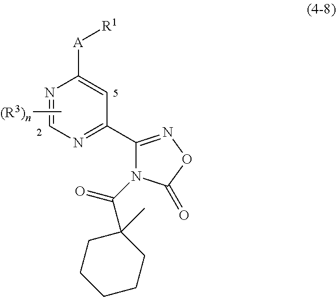

Compound represented by formula (4-9) (in which A, R¹ and R³ represent any one of combinations shown below).

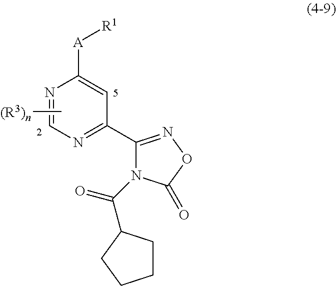

Compound represented by formula (4-10) (in which A, R¹ and R³ represent any one of combinations shown below).

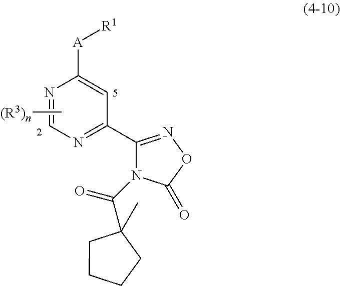

Compound represented by formula (4-11) (in which A, R¹ and R³ represent any one of combinations shown below).

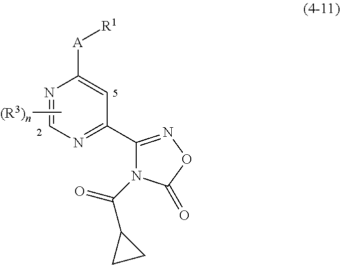

Compound represented by formula (4-12) (in which A, R¹ and R³ represent any one of combinations shown below).

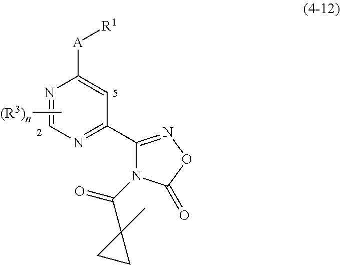

Compound represented by formula (4-13) (in which A, R¹ and R³ represent any one of combinations shown below).

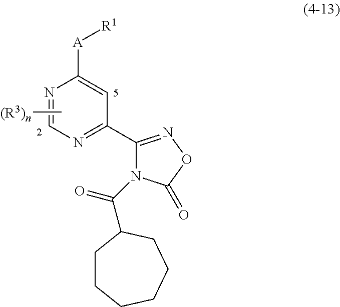

Compound represented by formula (4-14) (in which A, R¹ and R³ represent any one of combinations shown below).

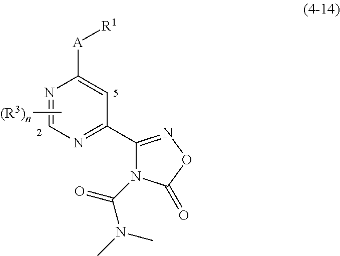

Compound represented by formula (4-15) (in which A, R¹ and R³ represent any one of combinations shown below).

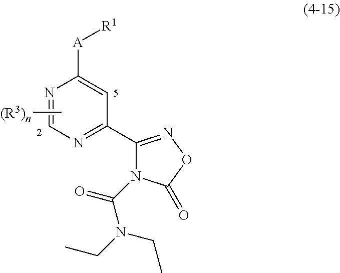

Compound represented by formula (4-16) (in which A, R¹ and R³ represent any one of combinations shown below).

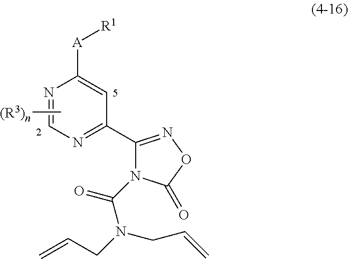

Compound represented by formula (4-17) (in which A, R¹ and R³ represent any one of combinations shown below).

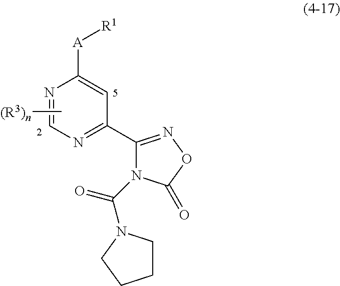

Compound represented by formula (4-18) (in which A, R¹ and R³ represent any one of combinations shown below).

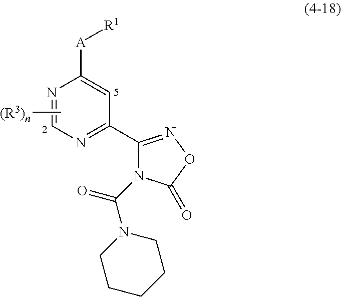

Compound represented by formula (4-19) (in which A, R¹ and R³ represent any one of combinations shown below).

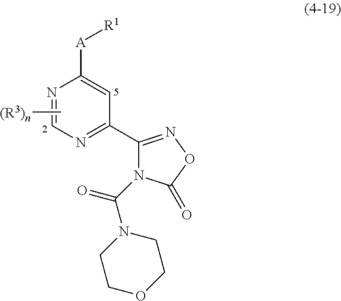

Compound represented by formula (4-20) (in which A, R¹ and R³ represent any one of combinations shown below).

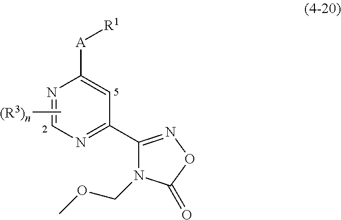

Compound represented by formula (4-21) (in which A, R¹ and R³ represent any one of combinations shown below).

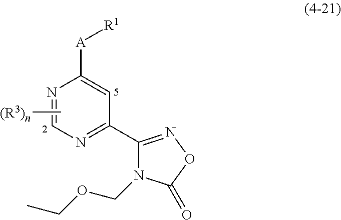

Compound represented by formula (4-22) (in which A, R¹ and R³ represent any one of combinations shown below).

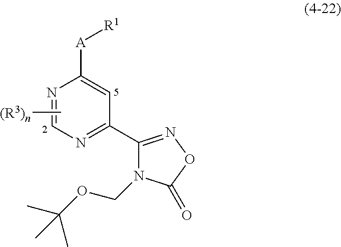

Compound represented by formula (4-23) (in which A, R¹ and R³ represent any one of combinations shown below).

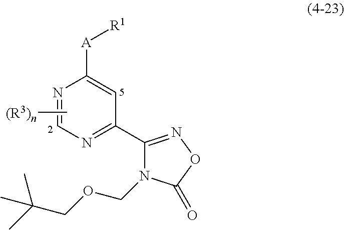

Compound represented by formula (4-24) (in which A, R¹ and R³ represent any one of combinations shown below).

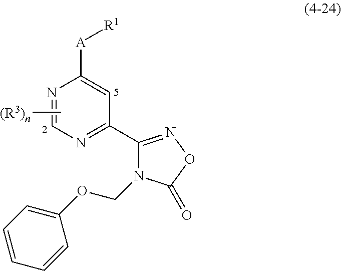

Compound represented by formula (4-25) (in which A, R¹ and R³ represent any one of combinations shown below).

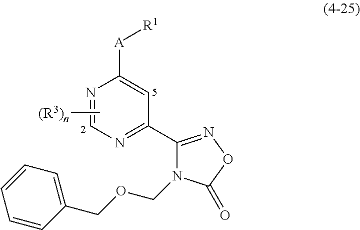

Compound represented by formula (4-26) (in which A, R¹ and R³ represent any one of combinations shown below).

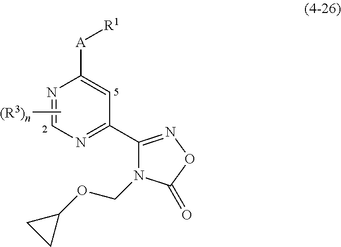

Compound represented by formula (4-27) (in which A, R¹ and R³ represent any one of combinations shown below).

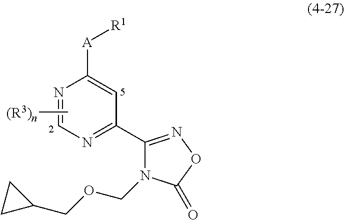

Compound represented by formula (4-28) (in which A, R¹ and R³ represent any one of combinations shown below).

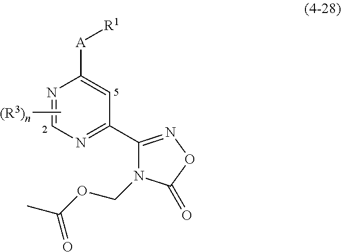

Compound represented by formula (4-29) (in which A, R¹ and R³ represent any one of combinations shown below).

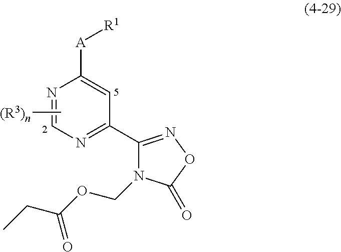

Compound represented by formula (4-30) (in which A, R¹ and R³ represent any one of combinations shown below).

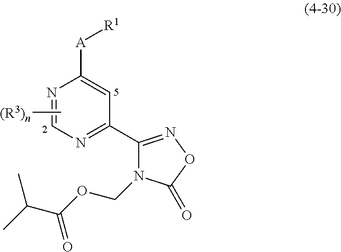

Compound represented by formula (4-31) (in which A, R¹ and R³ represent any one of combinations shown below).

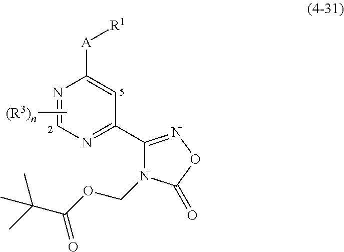

Compound represented by formula (4-32) (in which A, R¹ and R³ represent any one of combinations shown below).

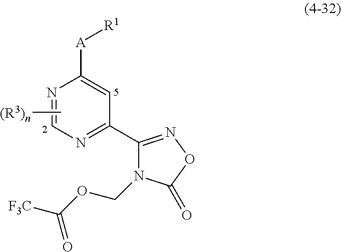

Compound represented by formula (4-33) (in which A, R¹ and R³ represent any one of combinations shown below).

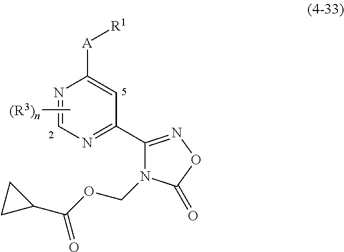

Compound represented by formula (4-34) (in which A, R¹ and R³ represent any one of combinations shown below).

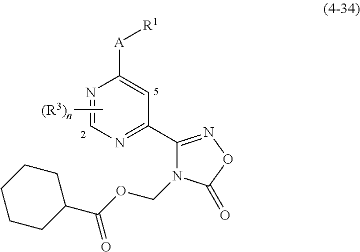

Compound represented by formula (4-35) (in which A, R¹ and R³ represent any one of combinations shown below).

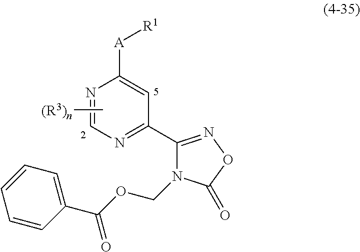

Compound represented by formula (4-36) (in which A, R¹ and R³ represent any one of combinations shown below).

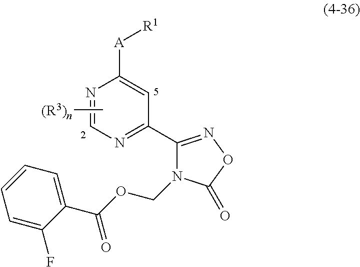

Compound represented by formula (4-37) (in which A, R¹ and R³ represent any one of combinations shown below).

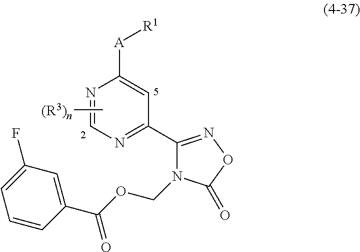

Compound represented by formula (4-38) (in which A, R¹ and R³ represent any one of combinations shown below).

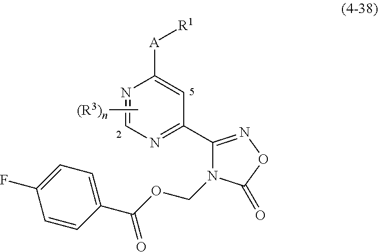

Compound represented by formula (4-39) (in which A, R¹ and R³ represent any one of combinations shown below).

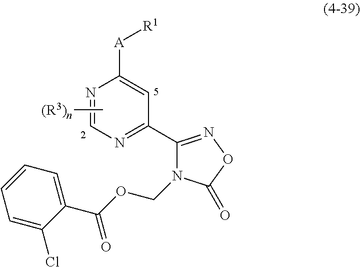

Compound represented by formula (4-40) (in which A, R¹ and R³ represent any one of combinations shown below).

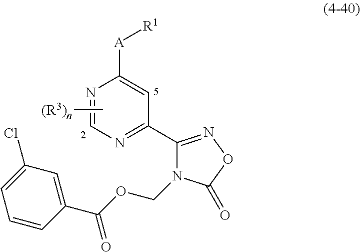

Compound represented by formula (4-41) (in which A, R¹ and R³ represent any one of combinations shown below).

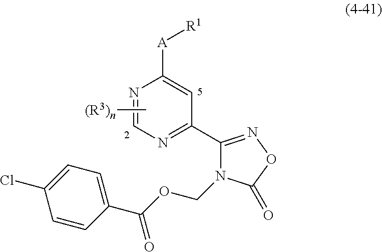

Compound represented by formula (4-42) (in which A, R¹ and R³ represent any one of combinations shown below).

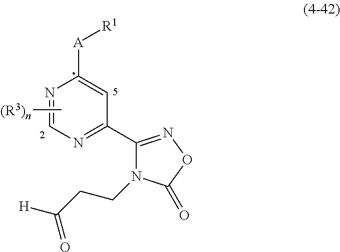

Combinations of A, R¹ and R³, when n is 0 or 1 in compounds represented by formulas (4-1) to (4-42), are shown below. In the following combinations, a branch number in parenthesis [ ], a group represented by A, a group represented by R¹ and a group represented by R³ are sequentially described.

For example, when n is 1, a combination is shown as follows: [31, a single bond, CF₃, 2-CH₃], which means a combination in which A is a single bond, R¹ is a CF₃ group, and R³ is a CH₃ group substituted at the 2-position of a pyrimidine ring. For example, when n is 0, a combination is shown as follows: [1, a single bond, CF₃, H], which means a combination in which A is a single bond, R¹ is a CF₃ group, and hydrogen is substituted at the 2- and 5-positions of a pyrimidine ring.
[branch number, A, R¹, R³]=[1, single bond, CF₃, H], [2, single bond, CHF₂, H], [3, single bond, CF₂CF₃, H], [4, single bond, CH₂CF₃, H], [5, single bond, CH(CH₂)CF₃, H], [6, single bond, CF(CF₃)₂, H], [7, single bond, CF₂CF₂CF₃, H], [8, single bond, CF₂CF₂CF₂CF₃, H], [9, single bond, CH₂CH₂CF₃, H], [10, single bond, CH(OCH₂)CF₃, H], [11, single bond, CH(OCH₂)CF₂CF₃, H], [12, single bond, CClF₂, H], [13, O, CF₃, H], [14, O, CH₂CF₃, H], [15, O, CH(CH₂)CF₃, H], [16, O, CF₂CF₃, H], [17, O, CClF₂, H], [18, O, CH₂CH₂CF₃, H], [19, S, CF₃, H], [20, S, CH₂CF₃, H], [21, S, CH(CH₂)CF₃, H], [22, S, CF₂CF₃, H], [23, S, CClF₂, H], [24, S, CH₂CH₂CF₃, H], [25, NCH₃, CF₃, H], [26, NCH₃, CH₂CF₃, H], [27, NCH₃, CH(CH₂)CF₃, H], [28, NCH₃, CF₂CF₃, H], [29, NCH₃, CClF₂, H], [30, NCH₃, CH₂CH₂CF₃, H], [31, single bond, CF₃, 2-CH₃], [32, single bond, CHF₂, 2-CH₃], [33, single bond, CF₂CF₃, 2-CH₃], [34, single bond, CH₂CF₃, 2-CH₃], [35, single bond, CH(CH₂)CF₃, 2-CH₃], [36, single bond, CF(CF₃)₂, 2-CH₃], [37, single bond, CF₂CF₂CF₃, 2-CH₃], [38, single bond, CF₂CF₂CF₂CF₃, 2-CH₃], [39, single bond, CH₂CH₂CF₃, 2-CH₃], [40, single bond, CH(OCH₂)CF₃, 2-CH₃], [41, single bond, CH(OCH₂)CF₂CF₃, 2-CH₃], [42, single bond, CClF₂, 2-CH₃], [43, O, CF₃, 2-CH₃], [44, O, CH₂CF₃, 2-CH₃], [45, O, CH(CH₂)CF₃, 2-CH₃], [46, O, CF₂CF₃, 2-CH₃], [47, O, CClF₂, 2-CH₃], [48, O, CH₂CH₂CF₃, 2-CH3], [49, S, CF₃, 2-CH₃], [50, S, CH₂CF₃, 2-CH₃], [51, S, CH(CH₂)CF₃, 2-CH₃], [52, S, CF₂CF₃, 2-CH₃], [53, S, CClF₂, 2-CH₃], [54, S, CH₂CH₂CF₃, 2-CH₃], [55, NCH₃, CF₃, 2-CH₃], [56, NCH₃, CH₂CF₃, 2-CH₃], [57, NCH₃, CH(CH₂)CF₃, 2-CH3], [58, NCH₃, CF₂CF₃, 2-CH₃], [59, NCH₃, CClF₂, 2-CH₃], [60, NCH₃, CH₂CH₂CF₃, 2-CH₃], [61, single bond, CF₃, 2-cyclopropyl], [62, single bond, CHF₂, 2-cyclopropyl], [63, single bond, CF₂CF₃, 2-cyclopropyl], [64, single bond, CH₂CF₃, 2-cyclopropyl], [65, single bond, CH(CH₂)CF₃, 2-cyclopropyl], [66, single bond, CF(CF₃)₂, 2-cyclopropyl], [67, single bond, CF₂CF₂CF₃, 2-cyclopropyl], [68, single bond, CF₂CF₂CF₂CF₃, 2-cyclopropyl], [69, single bond, CH₂CH₂CF₃, 2-cyclopropyl], [70, single bond, CH(OCH₂)CF₃, 2-cyclopropyl], [71, single bond, CH(OCH₂)CF₂CF₃, 2-cyclopropyl], [72, single bond, CClF₂, 2-cyclopropyl], [73, O, CF₃, 2-cyclopropyl], [74, O, CH₂CF₃, 2-cyclopropyl], [75, O, CH(CH₂)CF₃, 2-cyclopropyl], [76, O, CF₂CF₃, 2-cyclopropyl], [77, O, CClF₂, 2-cyclopropyl], [78, O, CH₂CH₂CF₃, 2-cyclopropyl], [79, S, CF₃, 2-cyclopropyl], [80, S, CH₂CF₃, 2-cyclopropyl], [81, S, CH(CH₂)CF₃, 2-cyclopropyl], [82, S, CF₂CF₃, 2-cyclopropyl], [83, S, CClF₂, 2-cyclopropyl], [84, S, CH₂CH₂CF₃, 2-cyclopropyl], [85, NCH₃, CF₃, 2-cyclopropyl], [86, NCH₃, CH₂CF₃, 2-cyclopropyl], [87, NCH₃, CH(CH₂)CF₃, 2-cyclopropyl], [88, NCH₃, CF₂CF₃, 2-cyclopropyl], [89, NCH₃, CClF₂, 2-cyclopropyl], [90, NCH₃, CH₂CH₂CF₃, 2-cyclopropyl], [91, single bond, CF₃, 2-CH₂CH₃], [92, single bond, CHF₂, 2-CH₂CH₃], [93, single bond, CF₂CF₃, 2-CH₂CH₃], [94, single bond, CH₂CF₃, 2-CH₂CH₃], [95, single bond, CH(CH₂)CF₃, 2-CH₂CH₃], [96, single bond, CF(CF₃)₂, 2-CH₂CH₃], [97, single bond, CF₂CF₂CF₃, 2-CH₂CH₃], [98, single bond, CF₂CF₂CF₂CF₃, 2-CH₂CH₃], [99, single bond, CH₂CH₂CF₃, 2-CH₂CH₃], [100, single bond, CH(OCH₂)CF₃, 2-CH₂CH₃],
[101, single bond, CH(OCH₂)CF₂CF₃, 2-CH₂CH₃], [102, single bond, CClF₂, 2-CH₂CH₃], [103, O, CF₃, 2-CH₂CH₃], [104, O, CH₂CF₃, 2-CH₂CH₃], [105, O, CH(CH₂)CF₃, 2-CH₂CH₃], [106, O, CF₂CF₃, 2-CH₂CH₃], [107, O, CClF₂, 2-CH₂CH₃], [108, O, CH₂CH₂CF₃, 2-CH₂CH₃], [109, S, CF₃, 2-CH₂CH₃], [110, S, CH₂CF₃, 2-CH₂CH₃], [111, S, CH(CH₂)CF₃, 2-CH₂CH₃], [112, S, CF₂CF₃, 2-CH₂CH₃], [113, S, CClF₂, 2-CH₂CH₃], [114, S, CH₂CH₂CF₃, 2-CH₂CH₃], [115, NCH₃, CF₃, 2-CH₂CH₃], [116, NCH₃, CH₂CF₃, 2-CH₂CH₃], [117, NCH₃, CH(CH₂)CF₃, 2-CH₂CH₃], [118, NCH₃, CF₂CF₃, 2-CH₂CH₃], [119, NCH₃, CClF₂, 2-CH₂CH₃], [120, NCH₃, CH₂CH₂CF₃, 2-CH₂CH₃], [121, single bond, CF₃, 2-CH(CH₃)₂], [122, single bond, CHF₂, 2-CH(CH₃)₂], [123, single bond, CF₂CF₃, 2-CH(CH₃)₂], [124, single bond, CH₂CF₃, 2-CH(CH₃)₂], [125, single bond, CH(CH₂)CF₃, 2-CH(CH₃)₂], [126, single bond, CF(CF₃)₂, 2-CH(CH₃)₂], [127, single bond, CF₂CF₂CF₃, 2-CH(CH₃)₂], [128, single bond, CF₂CF₂CF₂CF₃, 2-CH(CH₃)₂], [129, single bond, CH₂CH₂CF₃, 2-CH(CH₃)₂], [130, single bond, CH(OCH₂)CF₃, 2-CH(CH₃)₂], [131, single bond, CH(OCH₂)CF₂CF₃, 2-CH(CH₃)₂], [132, single bond, CClF₂, 2-CH(CH₃)₂], [133, O, CF₃, 2-CH(CH₃)₂], [134, O, CH₂CF₃, 2-CH(CH₃)₂], [135, O, CH(CH₂)CF₃, 2-CH(CH₃)₂], [136, O, CH₂CH₂CF₃, 2-CH(CH₃)₂], [137, S, CF₃, 2-CH(CH₃)₂], [138, NCH₃, CF₃, 2-CH(CH₃)₂], [139, NCH₃, CH₂CF₃, 2-CH(CH₃)₂], [140, NCH₃, CH(CH₂)CF₃, 2-CH(CH₃)₂], [141, NCH₃, CF₂CF₃, 2-CH(CH₃)₂], [142, single bond, CF₃, 2-C(CH₃)₃], [143, single bond, CHF₂, 2-C(CH₃)₃], [144, single bond, CF₂CF₃, 2-C(CH₃)₃], [145, single bond, CH₂CF₃, 2-C(CH₃)₃], [146, single bond, CH(CH₂)CF₃, 2-C(CH₃)₃], [147, single bond, CF(CF₃)₂, 2-C(CH₃)₃], [148, single bond, CF₂CF₂CF₃, 2-C(CH₃)₃], [149, single bond, CH₂CH₂CF₃, 2-C(CH₃)₃], [150, single bond, CH(OCH₂)CF₃, 2-C(CH₃)₃], [151, O, CF₃, 2-C(CH₃)₃], [152, O, CH₂CF₃, 2-C(CH₃)₃], [153, O, CH(CH₂)CF₃, 2-C(CH₃)₃], [154, O, CH₂CH₂CF₃, 2-C(CH₃)₃], [155, S, CF₃, 2-C(CH₃)₃], [156, NCH₃, CF₃, 2-C(CH₃)₃], [157, NCH₃, CH₂CF₃, 2-C(CH₃)₃], [158, single bond, CF₃, 2-OCH₂CF₃], [159, single bond, CHF₂, 2-OCH₂CF₃], [160, single bond, CF₂CF₃, 2-OCH₂CF₃], [161, single bond, CH₂CF₃, 2-OCH₂CF₃], [162, single bond, CH(CH₂)CF₃, 2-OCH₂CF₃], [163, single bond, CF(CF₃)₂, 2-OCH₂CF₃], [164, single bond, CF₂CF₂CF₃, 2-OCH₂CF₃], [165, single bond, CH(OCH₂)CF₃, 2-OCH₂CF₃], [166, O, CF₃, 2-OCH₂CF₃], [167, O, CH₂CF₃, 2-OCH₂CF₃], [168, O, CH(CH₂)CF₃, 2-OCH₂CF₃], [169, S, CF₃, 2-OCH₂CF₃], [170, NCH₃, CH₂CF₃, 2-OCH₂CF₃], [171, single bond, CF₃, 2-OCH(CH₃)CF₃], [172, single bond, CF₂CF₃, 2-OCH(CH₃)CF₃], [173, single bond, CH₂CF₃, 2-OCH(CH₃)CF₃], [174, single bond, CH(CH₂)CF₃, 2-OCH(CH₃)CF₃], [175, single bond, CF(CF₃)₂, 2-OCH(CH₃)CF₃], [176, single bond, CF₂CF₂CF₃, 2-OCH(CH₃)CF₃], [177, single bond, CH(OCH₂)CF₃, 2-OCH(CH₃)CF₃], [178, O, CH₂CF₃, 2-OCH(CH₃)CF₃], [179, O, CH(CH₂)CF₃, 2-OCH(CH₃)CF₃], [180, single bond, CF₃, 2-SCH₃], [181, single bond, CF₂CF₃, 2-SCH₃], [182, single bond, CH₂CF₃, 2-SCH₃], [183, single bond, CH(CH₂)CF₃, 2-SCH₃], [184, single bond, CF(CF₃)₂, 2-SCH₃], [185, single bond, CF₂CF₂CF₃, 2-SCH₃], [186, single bond, CH(OCH₂)CF₃, 2-SCH₃], [187, O, CH₂CF₃, 2-SCH₃], [188, O, CH(CH₂)CF₃, 2-SCH₃], [189, single bond, CF₃, 2-CF₃, [190, single bond, CF₂CF₃, 2-CF₃], [191, single bond, CH₂CF₃, 2-CF₃], [192, single bond, CH(CH₂)CF₃, 2-CF₃], [193, single bond, CF(CF₃)₂, 2-CF₃], [194, single bond, CF₂CF₂CF₃, 2-CF₃], [195, single bond, CH(OCH₂)CF₃, 2-CF₃], [196, O, CH₂CF₃, 2-CF₃], [197, O, CH(CH₂)CF₃, 2-CF₃], [198, single bond, CF₃, 2-SCF₃], [199, single bond, CF₂CF₃, 2-SCF₃], [200, single bond, CH₂CF₃, 2-SCF₃],
[201, single bond, CH(CH₂)CF₃, 2-SCF₃], [202, single bond, CF(CF₃)₂, 2-SCF₃], [203, single bond, CF₂CF₂CF₃, 2-SCF₃], [204, single bond, CH(OCH₂)CF₃, 2-SCF₃], [205, O, CH₂CF₃, 2-SCF₃], [206, O, CH(CH₂)CF₃, 2-SCF₃], [207, single bond, CF₃, 5-CH₃], [208, single bond, CF₂CF₃, 5-CH₃], [209, single bond, CH₂CF₃, 5-CH₃], [210, single bond, CH(CH₂)CF₃, 5-CH₃], [211, single bond, CF(CF₃)₂, 5-CH₃], [212, single bond, CF₂CF₂CF₃, 5-CH₃], [213, single bond, CH(OCH₂)CF₃, 5-CH₃], [214, O, CH₂CF₃, 5-CH₃], [215, O, CH(CH₂)CF₃, 5-CH₃], [216, single bond, CF₃, 5-OCH₃], [217, single bond, CF₂CF₃, 5-OCH₃], [218, single bond, CH₂CF₃, 5-OCH₃], [219, single bond, CH(CH₂)CF₃, 5-OCH₃], [220, single bond, CF(CF₃)₂, 5-OCH₃], [221, single bond, CF₂CF₂CF₃, 5-OCH₃], [222, single bond, CH(OCH₂)CF₃, 5-OCH₃], [223, O, CH₂CF₃, 5-OCH₃], [224, O, CH(CH₂)CF₃, 5-OCH₃], [225, single bond, CF₃, 5-F], [226, single bond, CF₂CF₃, 5-F], [227, single bond, CH₂CF₃, 5-F], [228, single bond, CH(CH₂)CF₃, 5-F], [229, single bond, CF(CF₃)₂, 5-F], [230, single bond, CF₂CF₂CF₃, 5-F], [231, single bond, CH(OCH₂)CF₃, 5-F], [232, O, CH₂CF₃, 5-F], [233, O, CH(CH₂)CF₃, 5-F], [234, single bond, CF₃, 5-Cl], [235, single bond, CF₂CF₃, 5-Cl], [236, single bond, CH₂CF₃, 5-Cl], [237, single bond, CH(CH₂)CF₃, 5-Cl], [238, single bond, CF(CF₃)₂, 5-Cl], [239, single bond, CF₂CF₂CF₃, 5-Cl], [240, single bond, CH(OCH₂)CF₃, 5-Cl], [241, O, CH₂CF₃, 5-Cl], [242, O, CH(CH₂)CF₃, 5-Cl], [243, single bond, CF₃, 5-Br], [244, single bond, CF₂CF₃, 5-Br], [245, single bond, CH₂CF₃, 5-Br], [246, single bond, CH(CH₂)CF₃, 5-Br], [247, single bond, CF(CF₃)₂, 5-Br], [248, single bond, CF₂CF₂CF₃, 5-Br], [249, single bond, CH(OCH₂)CF₃, 5-Br], [250, O, CH₂CF₃, 5-Br], [251, O, CH(CH₂)CF₃, 5-Br],
[252, single bond, Ph, H], [253, single bond, 2-F-Ph, H], [254, single bond, 3-F-Ph, H], [255, single bond, 4-F-Ph, H], [256, single bond, 2,4-diF-Ph, H], [257, single bond, 2,3-diF-Ph, H], [258, single bond, 2,6-diF-211, H], [259, single bond, 3,5-diF-Ph, H], [260, single bond, 2-CF₃-Ph, H], [261, single bond, 3-CF₃-Ph, H], [262, single bond, 4-CF₃-Ph, H], [263, single bond, 2-CF₂CF₃-Ph, H], [264, single bond, 3-CF₂CF₃-Ph, H], [265, single bond, 2-CF(CF₃)₂-Ph, H], [266, single bond, 3-CF(CF₃)₂, H], [267, single bond, 2-OCF₃-Ph, H], [268, single bond, 3-OCF₃-Ph, H], [269, single bond, 2-CH₂CF₃-Ph, H], [270, single bond, 3-CH₂CF₃-Ph, H], [271, single bond, 2-OCH₂CF₃-Ph, H], [272, single bond, 3-OCH₂CF₃-Ph, H], [273, single bond, 2-CF₃-3F-Ph, H], [274, single bond, 2-CF₃-4F-Ph, H], [275, single bond, 2-CF₃-5F-Ph, H], [276, single bond, 2-CF₃-6F-Ph, H], [277, single bond, 3-CF₃-2F-Ph, H], [278, single bond, 3-CF₃-4-F-Ph, H], [279, single bond, 3-CF₃-5-F-Ph, H], [280, single bond, 3-CF₃-6-F-Ph, H], [281, single bond, 4-CF₃-2-F-Ph, H], [282, single bond, 4-CF₃-3-F-Ph, H], [283, single bond, 2-CF₃-6Cl-Ph, H], [284, single bond, 2,4-bisCF₃-Ph, H], [285, single bond, 2,3-bisCF₃-Ph, H], [286, single bond, 2,6-bisCF₃-Ph, H], [287, single bond, 3,5-bisCF₃-Ph, H], [288, single bond, 2-Cl-Ph, H], [289, single bond, 2-CH₃-Ph, H], [290, CH₂, Ph, H], [291, CH₂, 2-F-Ph, H], [292, CH₂, 3-F-Ph, H], [293, CH₂, 2-CF₃-Ph, H], [294, CH₂, 3-CF₃-Ph, H], [295, CH₂, 2-CF₂CF₃-Ph, H], [296, CH₂, 3-CF₂CF₃-Ph, H], [297, CH₂, 2-OCF₃-Ph, H], [298, CH₂, 3-OCF₃-Ph, H], [299, CH₂, 2-OCH₂CF₃-Ph, H], [300, CH₂, 3-OCH₂CF₃-Ph, H],
[301, CH₂, 2-CF₃-3F-Ph, H], [302, CH₂, 2-CF₃-5F-Ph, H], [303, CH₂, 2-CF₃-6F-Ph, H], [304, CH₂, 3-CF₃-2F-Ph, H], [305, CH₂, 3-CF₃-4-F-Ph, H], [306, CH₂, 3-CF₃-5-F-Ph, H], [307, CH₂, 3-CF₃-6-F-Ph, H], [308, CH₂, Ph, 2-CH₃], [309, CH₂, 2-F-Ph, 2-CH₃], [310, CH₂, 3-F-Ph, 2-CH₃], [311, CH₂, 2-CF₃-Ph, 2-CH₃], [312, CH₂, 3-CF₃-Ph, 2-CH₃], [313, CH₂, 2-CF₂CF₃-Ph, 2-CH₃], [314, CH₂, 3-CF₂CF₃-Ph, 2-CH₃], [315, CH₂, 2-OCF₃-Ph, 2-CH₃], [316, CH₂, 3-OCF₃-Ph, 2-CH₃], [317, CH₂, 2-OCH₂CF₃-Ph, 2-CH₃], [318, CH₂, 3-OCH₂CF₃-Ph, 2-CH₃], [319, CH₂, Ph, 2-cyclopropyl], [320, CH₂, 2-F-Ph, 2-cyclopropyl], [321, CH₂, 3-F-Ph, 2-cyclopropyl], [322, CH₂, 2-CF₃-Ph, 2-cyclopropyl], [323, CH₂, 3-CF₃-Ph, 2-cyclopropyl], [324, CH₂, 2-CF₂CF₃-Ph, 2-cyclopropyl], [325, CH₂, 3-CF₂CF₃-Ph, 2-cyclopropyl], [326, CH₂, 2-OCF₃-Ph, 2-cyclopropyl], [327, CH₂, 3-OCF₃-Ph, 2-cyclopropyl], [328, CH₂, 2-OCH₂CF₃-Ph, 2-cyclopropyl], [329, CH₂, 3-OCH₂CF₃-Ph, 2-cyolopropyl], [330, CH₂, Ph, 2-CH₂CH₃], [331, CH₂, 2-F-Ph, 2-CH₂CH₃], [332, CH₂, 3-F-Ph, 2-CH₂CH₃], [333, CH₂, 2-CF₃-Ph, 2-CH₂CH₃], [334, CH₂, 3-CF₃-Ph, 2-CH₂CH₃], [335, CH₂, 2-CF₂CF₃-Ph, 2-CH₂CH₃], [336, CH₂, 3-CF₂CF₃-Ph, 2-CH₂CH₃], [337, CH₂, 2-OCF₃-Ph, 2-CH₂CH₃], [338, CH₂, 3-OCF₃-Ph, 2-CH₂CH₃], [339, CH₂, 2-OCH₂CF₃-Ph, 2-CH₂CH₃], [340, CH₂, 3-OCH₂CF₃-Ph, 2-CH₂CH₃], [341, CH₂, Ph, 2-CF₃], [342, CH₂, 2-F-Ph, 2-CF₃], [343, CH₂, 3-F-Ph, 2-CF₃], [344, CH₂, 2-CF₃-Ph, 2-CF₃], [345, CH₂, 3-CF₃-Ph, 2-CF₃], [346, CH₂, 2-CF₂CF₃-Ph, 2-CF₃], [347, CH₂, 3-CF₂CF₃-Ph, 2-CF₃], [348, CH₂, 2-OCF₃-Ph, 2-CF₃], [349, CH₂, 3-OCF₃-Ph, 2-CF₃], [350, CH₂, 2-OCH₂CF₃-Ph, 2-CF₃], [351, CH₂, 3-OCH₂CF₃-Ph, 2-CF₃], [352, CH₂, Ph, 2-OCH₃], [353, CH₂, 2-F-Ph, 2-OCH₃], [354, CH₂, 3-F-Ph, 2-OCH₃], [355, CH₂, 2-CF₃-Ph, 2-OCH₃], [356, CH₂, 3-CF₃-Ph, 2-OCH₃], [357, CH₂, 2-CF₂CF₃-Ph, 2-OCH₃], [358, CH₂, 3-CF₂CF₃-Ph, 2-OCH₃], [359, CH₂, 2-OCF₃-Ph, 2-OCH₃], [360, CH₂, 3-OCF₃-Ph, 2-OCH₃], [361, CH₂, 2-OCH₂CF₃-Ph, 2-OCH₃], [362, CH₂, 3-OCH₂CF₃-Ph, 2-OCH₃], [363, CH₂, Ph, 5-CH₃], [364, CH₂, 2-F-Ph, 5-CH₃], [365, CH₂, 3-F-Ph, 5-CH₃], [366, CH₂, 2-CF₃-Ph, 5-CH₃], [367, CH₂, 3-CF₃-Ph, 5-CH₃], [368, CH₂, 2-CF₂CF₃-Ph, 5-CH₃], [369, CH₂, 3-CF₂CF₃-Ph, 5-CH₃], [370, CH₂, 2-OCF₃-Ph, 5-CH₃], [371, CH₂, 3-OCF₃-Ph, 5-CH₃], [372, CH₂, 2-OCH₂CF₃-Ph, 5-CH₃], [373, CH₂, 3-OCH₂CF₃-Ph, 5-CH₃], [374, O, Ph, H], [375, O, 2-F-Ph, H], [376, O, 3-F-Ph, H], [377, O, 2-CF₃-Ph, H], [378, O, 3-CF₃-Ph, H], [379, O, 2-CF₂CF₃-Ph, H], [380, O, 3-CF₂CF₃-Ph, H], [381, O, 2-OCF₃-Ph, H], [382, O, 3-OCF₃-Ph, H], [383, O, 2-OCH₂CF₃-Ph, H], [384, O, 3-OCH₂CF₃-Ph, H], [385, O, Ph, 2-cyclopropyl], [386, O, 2-F-Ph, 2-cyclopropyl], [387, O, 3-F-Ph, 2-cyclopropyl], [388, O, 2-CF₃-Ph, 2-cyclopropyl], [389, O, 3-CF₃-Ph, 2-cyclopropyl], [390, O, 2-CF₂CF₃-Ph, 2-cyclopropyl], [391, O, 3-CF₂CF₃-Ph, 2-cyclopropyl], [392, O, 2-OCF₃-Ph, 2-cyclopropyl], [393, O, 3-OCF₃-Ph, 2-cyclopropyl], [394, O, 2-OCH₂CF₃-Ph, 2-cyclopropyl], [395, O, 3-OCH₂CF₃-Ph, 2-cyclopropyl], [396, O, Ph, 2-OCH₃], [397, O, 2-F-Ph, 2-OCH₃], [398, O, 3-F-Ph, 2-OCH₃], [399, O, 2-CF₃-Ph, 2-OCH₃], [400, O, 3-CF₃-Ph, 2-OCH₃],
[401, O, 2-CF₂CF₃-Ph, 2-OCH₃], [402, O, 3-CF₂CF₃-Ph, 2-OCH₃], [403, O, 2-OCF₃-Ph, 2-OCH₃], [404, O, 3-OCF₃-Ph, 2-OCH₃], [405, O, 2-OCH₂CF₃-Ph, 2-OCH₃], [406, O, 3-OCH₂CF₃-Ph, 2-OCH₃], [407, NCH₃, Ph, H], [408, NCH₃, 2-F-Ph, H], [409, NCH₃, 3-F-Ph, H], [410, NCH₃, 2-CF₃-Ph, H], [411, NCH₃, 3-CF₃-Ph, H], [412, NCH₃, 2-CF₂CF₃-Ph, H], [413, NCH₃, 3-CF₂CF₃-Ph, H], [414, NCH₃, 2-OCF₃-Ph, H], [415, NCH₃, 3-OCF₃-Ph, H], [416, NCH₃, 2-OCH₂CF₃-Ph, H], [417, NCH₃, 3-OCH₂CF₃-Ph, H], [418, NCH₃, Ph, 2-cyclopropyl], [419, NCH₃, 2-F-Ph, 2-cyclopropyl], [420, NCH₃, 3-F-Ph, 2-cyclopropyl], [421, NCH₃, 2-CF₃-Ph, 2-cyclopropyl], [422, NCH₃, 3-CF₃-Ph, 2-cyclopropyl], [423, NCH₃, 2-CF₂CF₃-Ph, 2-cyclopropyl], [424, NCH₃, 3-CF₂CF₃-Ph, 2-cyclopropyl], [425, NCH₃, 2-OCF₃-Ph, 2-cyclopropyl], [426, NCH₃, 3-OCF₃-Ph, 2-cyclopropyl], [427, NCH₃, 2-OCH₂CF₃-Ph, 2-cyclopropyl], [428, NCH₃, 3-OCH₂CF₃-Ph, 2-cyclopropyl], [429, NCH₃, Ph, 2-OCH₃], [430, NCH₃, 2-F-Ph, 2-OCH₃], [431, NCH₃, 3-F-Ph, 2-OCH₃], [432, NCH₃, 2-CF₃-Ph, 2-OCH₃], [433, NCH₃, 3-CF₃-Ph, 2-OCH₃], [434, single bond, Ph, 2-CH₃], [435, single bond, 2-F-Ph, 2-CH₃], [436, single bond, 3-F-Ph, 2-CH₃], [437, single bond, 2-CF₃-Ph, 2-CH₃], [438, single bond, 3-CF₃-Ph, 2-CH₃], [439, single bond, 2-CF₂CF₃-Ph, 2-CH₃], [440, single bond, 3-CF₂CF₃-Ph, 2-CH₃], [441, single bond, 2-OCF₃-Ph, 2-CH₃], [442, single bond, 3-OCF₃-Ph, 2-CH₃], [443, single bond, 2-OCH₂CF₃-Ph, 2-CH₃], [444, single bond, 3-OCH₂CF₃-Ph, 2-CH₃], [445, single bond, Ph, 2-cyclopropyl], [446, single bond, 2-F-Ph, 2-cyclopropyl], [447, single bond, 3-F-Ph, 2-cyclopropyl], [448, single bond, 2-CF₃-Ph, 2-cyclopropyl], [449, single bond, 3-CF₃-Ph, 2-cyclopropyl], [450, single bond, 2-CF₂CF₃-Ph, 2-cyclopropyl], [451, single bond, 3-CF₂CF₃-Ph, 2-cyclopropyl], [452, single bond, 2-OCF₃-Ph, 2-cyclopropyl], [453, single bond, 3-OCF₃-Ph, 2-cyclopropyl], [454, single bond, 2-OCH₂CF₃-Ph, 2-cyclopropyl], [455, single bond, 3-OCH₂CF₃-Ph, 2-cyclopropyl], [456, single bond, Ph, 2-CH₂CH₃], [457, single bond, 2-F-Ph, 2-CH₂CH₃], [458, single bond, 3-F-Ph, 2-CH₂CH₃], [459, single bond, 2-CF₃-Ph, 2-CH₂CH₃], [460, single bond, 3-CF₃-Ph, 2-CH₂CH₃], [461, single bond, 2-CF₂CF₃-Ph, 2-CH₂CH₃], [462, single bond, 3-CF₂CF₃-Ph, 2-CH₂CH₃], [463, single bond, 2-OCF₃-Ph, 2-CH₂CH₃], [464, single bond, 3-OCF₃-Ph, 2-CH₂CH₃], [465, single bond, 2-OCH₂CF₃-Ph, 2-CH₂CH₃], [466, single bond, 3-OCH₂CF₃-Ph, 2-CH₂CH₃], [467, single bond, Ph, 2-CF₃], [468, single bond, 2-F-Ph, 2-CF₃], [469, single bond, 3-F-Ph, 2-CF₃], [470, single bond, 2-CF₃-Ph, 2-CF₃], [471, single bond, 3-CF₃-Ph, 2-CF₃], [472, single bond, 2-CF₂CF₃-Ph, 2-CF₃], [473, single bond, 3-CF₂CF₃-Ph, 2-CF₃], [474, single bond, 2-OCF₃-Ph, 2-CF₃], [475, single bond, 3-OCF₃-Ph, 2-CF₃], [476, single bond, 2-OCH₂CF₃-Ph, 2-CF₃], [477, single bond, 3-OCH₂CF₃-Ph, 2-CF₃], [478, single bond, Ph, 2-OCH₃], [479, single bond, 2-F-Ph, 2-OCH₃], [480, single bond, 3-F-Ph, 2-OCH₃], [481, single bond, 2-CF₃-Ph, 2-OCH₃], [482, single bond, 3-CF₃-Ph, 2-OCH₃], [483, single bond, 2-CF₂CF₃-Ph, 2-OCH₃], [484, single bond, 3-CF₂CF₃-Ph, 2-OCH₃], [485, single bond, 2-OCF₃-Ph, 2-OCH₃], [486, single bond, 3-OCF₃-Ph, 2-OCH₃], [487, single bond, 2-OCH₂CF₃-Ph, 2-OCH₃], [488, single bond, 3-OCH₂CF₃-Ph, 2-OCH₃], [489, single bond, Ph, 5-CH₃], [490, single bond, 2-F-Ph, 5-CH₃], [491, single bond, 3-F-Ph, 5-CH₃], [492, single bond, 2-CF₃-Ph, 5-CH₃], [493, single bond, 3-CF₃-Ph, 5-CH₃], [494, single bond, 2-CF₂CF₃-Ph, 5-CH₃], [495, single bond, 3-CF₂CF₃-Ph, 5-CH₃], [496, single bond, 2-OCF₃-Ph, 5-CH₃], [497, single bond, 3-OCF₃-Ph, 5-CH₃], [498, single bond, 2-OCH₂CF₃-Ph, 5-CH₃], [499, single bond, 3-OCH₂CF₃-Ph, 5-CH₃], [500, OCH₂, Ph, H],
[501, OCH₂, 2-F-Ph, H], [502, OCH₂, 3-F-Ph, H], [503, OCH₂, 2-CF₃-Ph, H], [504, OCH₂, 3-CF₃-Ph, H], [505, OCH₂, Ph, 2-CH₃], [506, OCH₂, 2-F-Ph, 2-CH₃], [507, OCH₂, 3-F-Ph, 2-CH₃], [508, OCH₂, 2-CF₃-Ph, 2-CH₃], [509, OCH₂, 3-CF₃-Ph, 2-CH₃], [510, OCH₂, Ph, 2-cyclopropyl], [511, OCH₂, 2-CF₃-Ph, 2-cyclopropyl], [512, OCH₂, 3-CF₃-Ph, 2-cyclopropyl], [513, OCH₂, 2-OCF₃-Ph, 2-cyclopropyl], [514, OCH₂, 3-OCF₃-Ph, 2-cyclopropyl], [515, OCH₂, Ph, 2-CF₃], [516, OCH₂, 3-F-Ph, 2-CF₃], [517, OCH₂, 2-CF₃-Ph, 2-CF₃], [518, OCH₂, 3-CF₃-Ph, 2-CF₃], [519, OCH₂, Ph, 2-OCH₃], [520, OCH₂, 2-F-Ph, 2-OCH₃], [521, OCH₂, 3-F-Ph, 2-OCH₃], [522, OCH₂, 2-CF₃-Ph, 2-OCH₃], [523, OCH₂, 3-CF₃-Ph, 2-OCH₃], [524, OCH₂, Ph, 5-CH₃], [525, OCH₂, 2-F-Ph, 5-CH₃], [526, OCH₂, 3-F-Ph, 5-CH₃], [527, OCH₂, 2-CF₃-Ph, 5-CH₃], [528, OCH₂, 3-CF₃-Ph, 5-CH₃], [529, single bond, 2-py, H], [530, single bond, 3-CF₃-2-py, H], [531, single bond, 6-CF₃-2-py, H], [532, single bond, 2-py, 2-CH₃], [533, single bond, 2-py, 2-cyclopropyl], [534, single bond, 2-py, 2-CF₃], [535, single bond, 2-py, 2-OCH₃], [536, single bond, 2-py, 5-CH₃], [537, single bond, 3-py, H], [538, single bond, 2-CF₃-3-py, H], [539, single bond, 6-CF₃-3-py, H], [540, single bond, 3-py, 2-CH₃], [541, single bond, 3-py, 2-cyclopropyl], [542, single bond, 3-py, 2-CF₃], [543, single bond, 3-py, 2-OCH₃], [544, single bond, 3-py, 5-CH₃], [545, single bond, 4-py, H], [546, single bond, 2-CF₃-4-py, H], [547, single bond, 4-py, 2-CH₃], [548, single bond, 4-py, 2-cyclopropyl], [549, single bond, 4-py, 2-CF₃], [550, single bond, 4-py, 2-OCH₃], [551, single bond, 4-py, 5-CH₃].

Combinations of A, R¹ and R³, when n is 2, in compounds represented by formulas (4-1) to (4-42), are shown below. In the following combinations, a branch number in parenthesis [ ], a group represented by A, a group represented by R¹ and two groups represented by R³ are sequentially described. For example, a combination is shown as follows: [552, a single bond, CF₃, 2-CH₃, 5-CH₃], which means a combination in which A is a single bond, R¹ is a CF₃ group, and R³ is a CH₃ group substituted at the 2-position of a pyrimidine ring and a CH₃ group substituted at the 5-position.
[552, single bond, CF₃, 2-CH₃, 5-CH₃], [553, single bond, CF₂CF₃, 2-CH₃, 5-CH₃], [554, single bond, CH₂CF₃, 2-CH₃, 5-CH₃], [555, single bond, CH(CH₂)CF₃, 2-CH₃, 5-CH₃], [556, single bond, CF(CF₃)₂, 2-CH₃, 5-CH₃], [557, single bond, CF₂CF₂CF₃, 2-CH₃, 5-CH₃], [558, single bond, CF₂CF₂CF₂CF₃, 2-CH₃, 5-CH₃], [559, single bond, CH₂CH₂CF₃, 2-CH₃, 5-CH₃], [560, single bond, CH(OCH₂)CF₃, 2-CH₃, 5-CH₃], [561, single bond, CF₃, 2-CF₃, 5-CH₃], [562, single bond, CF₂CF₃, 2-CF₃, 5-CH₃], [563, single bond, CH₂CF₃, 2-CF₃, 5-CH₃], [564, single bond, CH(CH₂)CF₃, 2-CF₃, 5-CH₃], [565, single bond, CF(CF₃)₂, 2-CF₃, 5-CH₃], [566, single bond, CF₂CF₂CF₃, 2-CF₃, 5-CH₃], [567, single bond, CF₂CF₂CF₂CF₃, 2-CF₃, 5-CH₃], [568, single bond, CH₂CH₂CF₃, 2-CF₃, 5-CH₃], [569, single bond, CH(OCH₂)CF₃, 2-CF₃, 5-CH₃], [570, single bond, CF₃, 2-CH₃, 5-CF₃], [571, single bond, CF₂CF₃, 2-CH₃, 5-CF₃], [572, single bond, CH₂CF₃, 2-CH₃, 5-CF₃], [573, single bond, CH(CH₂)CF₃, 2-CH₃, 5-CF₃], [574, single bond, CF(CF₃)₂, 2-CH₃, 5-CF₃], [575, single bond, CF₂CF₂CF₃, 2-CH₃, 5-CF₃], [576, single bond, CF₂CF₂CF₂CF₃, 2-CH₃, 5-CF₃], [577, single bond, CH₂CH₂CF₃, 2-CH₃, 5-CF₃], [578, single bond, CH(OCH₂)CF₃, 2-CH₃, 5-CF₃], [579, single bond, CF₃, 2-CH₃, 5-F], [580, single bond, CF₂CF₃, 2-CH₃, 5-F], [581, single bond, CH₂CF₃, 2-CH₃, 5-F], [582, single bond, CH(CH₂)CF₃, 2-CH₃, 5-F], [583, single bond, CF(CF₃)₂, 2-CH₃, 5-F], [584, single bond, CF₂CF₂CF₃, 2-CH₃, 5-F], [585, single bond, CF₂CF₂CF₂CF₃, 2-CH₃, 5-F], [586, single bond, CH₂CH₂CF₃, 2-CH₃, 5-F], [587, single bond, CH(OCH₂)CF₃, 2-CH₃, 5-F], [588, single bond, CF₃, 2-CH₃, 5-Cl], [589, single bond, CF₂CF₃, 2-CH₃, 5-Cl], [590, single bond, CH₂CF₃, 2-CH₃, 5-Cl], [591, single bond, CH(CH₂)CF₃, 2-CH₃, 5-Cl], [592, single bond, CF(CF₃)₂, 2-CH₃, 5-Cl], [593, single bond, CF₂CF₂CF₃, 2-CH₃, 5-Cl], [594, single bond, CF₂CF₂CF₂CF₃, 2-CH₃, 5-Cl], [595, single bond, CH₂CH₂CF₃, 2-CH₃, 5-Cl], [596, single bond, CH(OCH₂)CF₃, 2-CH₃, 5-Cl], [597, single bond, CF₃, 2-cyclopropyl, 5-CH₃], [598, single bond, CF₂CF₃, 2-cyclopropyl, 5-CH₃], [599, single bond, CH₂CF₃, 2-cyclopropyl, 5-CH₃], [600, single bond, CH(CH₂)CF₃, 2-cyclopropyl, 5-CH₃], [601, single bond, CF(CF₃)₂, 2-cyclopropyl, 5-CH₃], [602, single bond, CF₂CF₂CF₃, 2-cyclopropyl, 5-CH₃], [603, single bond, CF₂CF₂CF₂CF₃, 2-cyclopropyl, 5-CH₃], [604, single bond, CH₂CH₂CF₃, 2-cyclopropyl, 5-CH₃], [605, single bond, CH(OCH₂)CF₃, 2-cyclopropyl, 5-CH₃], [606, O, CF₃, 2-CH₃, 5-CH₃], [607, O, CH₂CF₃, 2-CH₃, 5-CH₃], [608, O, CH(CH₂)CF₃, 2-CH₃, 5-CH₃], [609, O, CF₃, 2-CF₃, 5-CH₃][610, O, CH₂CF₃, 2-CF₃, 5-CH₃], [611, O, CH(CH₂)CF₃, 2-CF₃, 5-CH₃], [612, O, CF₃, 2-CH₃, 5-CF₃], [613, O, CH₂CF₃, 2-CH₃, 5-CF₃], [614, O, CH(CH₂)CF₃, 2-CH₃, 5-CF₃], [615, O, CF₃, 2-CH₃, 5-F], [616, O, CH₂CF₃, 2-CH₃, 5-F], [617, O, CH(CH₂)CF₃, 2-CH₃, 5-F], [618, O, CF₃, 2-CH₃, 5-Cl], [619, O, CH₂CF₃, 2-CH₃, 5-Cl], [620, O, CH(CH₂)CF₃, 2-CH₃, 5-Cl], [621, O, CF₃, 2-cyclopropyl, 5-CH₃], [622, O, CH₂CF₃, 2-cyclopropyl, 5-CH₃], [623, O, CH(CH₂)CF₃, 2-cyclopropyl, 5-CH₃], [624, single bond, Ph, 2-CH₃, 5-CH₃], [625, single bond, 2-F-Ph, 2-CH₃, 5-CH₃], [626, single bond, 3-F-Ph, 2-CH₃, 5-CH₃], [627, single bond, 2-CF₃-Ph, 2-CH₃, 5-CH₃], [628, single bond, 3-CF₃-Ph, 2-CH₃, 5-CH₃], [629, single bond, 2-CF₂CF₃-Ph, 2-CH₃, 5-CH₃], [630, single bond, 3-CF₂CF₃-Ph, 2-CH₃, 5-CH₃], [631, single bond, 2-OCF₃-Ph, 2-CH₃, 5-CH₃], [632, single bond, 3-OCF₃-Ph, 2-CH₃, 5-CH₃], [633, single bond, 2-OCH₂CF₃-Ph, 2-CH₃, 5-CH₃], [634, single bond, 3-OCH₂CF₃-Ph, 2-CH₃, 5-CH₃], [635, single bond, Ph, 2-CF₃, 5-CH₃], [636, single bond, 2-F-Ph, 2-CF₃, 5-CH₃], [637, single bond, 3-F-Ph, 2-CF₃, 5-CH₃], [638, single bond, 2-CF₃-Ph, 2-CF₃, 5-CH₃], [639, single bond, 3-CF₃-Ph, 2-CF₃, 5-CH₃], [640, single bond, 2-CF₂CF₃-Ph, 2-CF₃, 5-CH₃], [641, single bond, 3-CF₂CF₃-Ph, 2-CF₃, 5-CH₃], [642, single bond, 2-OCF₃-Ph, 2-CF₃, 5-CH₃], [643, single bond, 3-OCF₃-Ph, 2-CF₃, 5-CH₃], [644, single bond, 2-OCH₂CF₃-Ph, 2-CF₃, 5-CH₃], [645, single bond, 3-OCH₂CF₃-Ph, 2-CF₃, 5-CH₃], [646, single bond, Ph, 2-CH₃, 5-Cl], [647, single bond, 2-F-Ph, 2-CH₃, 5-Cl], [648, single bond, 3-F-Ph, 2-CH₃, 5-Cl], [649, single bond, 2-CF₃-Ph, 2-CH₃, 5-Cl], [650, single bond, 3-CF₃-Ph, 2-CH₃, 5-Cl], [651, single bond, 2-CF₂CF₃-Ph, 2-CH₃, 5-Cl], [652, single bond, 3-CF₂CF₃-Ph, 2-CH₃, 5-Cl].
[653, single bond, 2-OCF₃-Ph, 2-CH₃, 5-Cl], [654, single bond, 3-OCF₃-Ph, 2-CH₃, 5-Cl], [655, single bond, 2-OCH₂CF₃-Ph, 2-CH₃, 5-Cl][656, single bond, 3-OCH₂CF₃-Ph, 2-CH₃, 5-Cl], [657, single bond, Ph, 2-CH₃, 5-F], [658, single bond, 2-F-Ph, 2-CH₃, 5-F], [659, single bond, 3-F-Ph, 2-CH₃, 5-F], [660, single bond, 2-CF₃-Ph, 2-CH₃, 5-F], [661, single bond, 3-CF₃-Ph, 2-CH₃, 5-F], [662, single bond, 2-CF₂CF₃-Ph, 2-CH₃, 5-F], [663, single bond, 3-CF₂CF₃-Ph, 2-CH₃, 5-F], [664, single bond, 2-OCF₃-Ph, 2-CH₃, 5-F], [665, single bond, 3-OCF₃-Ph, 2-CH₃, 5-F], [666, single bond, 2-OCH₂CF₃-Ph, 2-CH₃, 5-F], [667, single bond, 3-OCH₂CF₃-Ph, 2-CH₃, 5-F], [668, single bond, Ph, 2-cyclopropyl, 5-CH₃], [669, single bond, 2-F-Ph, 2-cyclopropyl, 5-CH₃], [670, single bond, 3-F-Ph, 2-cyclopropyl, 5-CH₃], [671, single bond, 2-CF₃-Ph, 2-cyclopropyl, 5-CH₃], [672, single bond, 3-CF₃-Ph, 2-cyclopropyl, 5-CH₃], [673, single bond, 2-CF₂CF₃-Ph, 2-cyclopropyl, 5-CH₃], [674, single bond, 3-CF₂CF₃-Ph, 2-cyclopropyl, 5-CH₃], [675, single bond, 2-OCF₃-Ph, 2-cyclopropyl, 5-CH₃], [676, single bond, 3-OCF₃-Ph, 2-cyclopropyl, 5-CH₃], [677, single bond, 2-OCH₂CF₃-Ph, 2-cyclopropyl, 5-CH₃], [678, single bond, 3-OCH₂CF₃-Ph, 2-cyclopropyl, 5-CH₃], [679, O, Ph, 2-CH₃, 5-CH₃], [680, O, 2-F-Ph, 2-CH₃, 5-CH₃], [681, O, 3-F-Ph, 2-CH₃, 5-CH₃], [682, O, 2-CF₃-Ph, 2-CH₃, 5-CH₃], [683, O, 3-CF₃-Ph, 2-CH₃, 5-CH₃], [684, O, 2-CF₂CF₃-Ph, 2-CH₃, 5-CH₃], [685, O, 3-CF₂CF₃-Ph, 2-CH₃, 5-CH₃], [686, O, 2-OCF₃-Ph, 2-CH₃, 5-CH₃], [687, O, 3-OCF₃-Ph, 2-CH₃, 5-CH₃], [688, O, 2-OCH₂CF₃-Ph, 2-CH₃, 5-CH₃], [689, O, 3-OCH₂CF₃-Ph, 2-CH₃, 5-CH₃], [690, O, Ph, 2-CF₃, 5-CH₃], [691, O, 2-F-Ph, 2-CF₃, 5-CH₃], [692, O, 3-F-Ph, 2-CF₃, 5-CH₃], [693, O, 2-CF₃-Ph, 2-CF₃, 5-CH₃], [694, O, 3-CF₃-Ph, 2-CF₃, 5-CH₃], [695, O, 2-CF₂CF₃-Ph, 2-CF₃, 5-CH₃], [696, O, 3-CF₂CF₃-Ph, 2-CF₃, 5-CH₃], [697, O, 2-OCF₃-Ph, 2-CF₃, 5-CH₃], [698, O, 3-OCF₃-Ph, 2-CF₃, 5-CH₃], [699, O, 2-OCH₂CF₃-Ph, 2-CF₃, 5-CH₃], [700, O, 3-OCH₂CF₃-Ph, 2-CF₃, 5-CH₃], [701, O, Ph, 2-CH₃, 5-Cl], [702, O, 2-F-Ph, 2-CH₃, 5-Cl], [703, O, 3-F-Ph, 2-CH₃, 5-Cl], [704, O, 2-CF₃-Ph, 2-CH₃, 5-Cl], [705, O, 3-CF₃-Ph, 2-CH₃, 5-Cl], [706, O, 2-CF₂CF₃-Ph, 2-CH₃, 5-Cl], [707, O, 3-CF₂CF₃-Ph, 2-CH₃, 5-Cl] [708, O, 2-OCF₃-Ph, 2-CH₃, 5-Cl], [709, O, 3-OCF₃-Ph, 2-CH₃, 5-Cl], [710, O, 2-OCH₂CF₃-Ph, 2-CH₃, 5-Cl][711, O, 3-OCH₂CF₃-Ph, 2-CH₃, 5-Cl], [712, O, Ph, 2-CH₃, 5-F], [713, O, 2-F-Ph, 2-CH₃, 5-F], [714, O, 3-F-Ph, 2-CH₃, 5-F], [715, O, 2-CF₃-Ph, 2-CH₃, 5-F], [716, O, 3-CF₃-Ph, 2-CH₃, 5-F], [717, O, 2-CF₂CF₃-Ph, 2-CH₃, 5-F], [718, O, 3-CF₂CF₃-Ph, 2-CH₃, 5-F], [719, O, 2-OCF₃-Ph, 2-CH₃, 5-F], [720, O, 3-OCF₃-Ph, 2-CH₃, 5-F], [721, O, 2-OCH₂CF₃-Ph, 2-CH₃, 5-F], [722, O, 3-OCH₂CF₃-Ph, 2-CH₃, 5-F], [723, O, Ph, 2-cyclopropyl, 5-CH₃], [724, O, 2-F-Ph, 2-cyclopropyl, 5-CH₃], [725, O, 3-F-Ph, 2-cyclopropyl, 5-CH₃], [726, O, 2-CF₃-Ph, 2-cyclopropyl, 5-CH₃], [727, O, 3-CF₃-Ph, 2-cyclopropyl, 5-CH₃], [728, O, 2-CF₂CF₃-Ph, 2-cyclopropyl, 5-CH₃], [729, O, 3-CF₂CF₃-Ph, 2-cyclopropyl, 5-CH₃], [730, O, 2-OCF₃-Ph, 2-cyclopropyl, 5-CH₃], [731, O, 3-OCF₃-Ph, 2-cyclopropyl, 5-CH₃], [732, O, 2-OCH₂CF₃-Ph, 2-cyclopropyl, 5-CH₃], [733, O, 3-OCH₂CF₃-Ph, 2-cyclopropyl, 5-CH₃], [734, single bond, 2-py, 2-CH₃, 5-CH₃], [735, single bond, 3-CF₃-2-py, 2-CH₃, 5-CH₃], [736, single bond, 2-py, 2-CF₃, 5-CH₃], [737, single bond, 3-CF₃-2-py, 2-CF₃, 5-CH₃], [738, single bond, 2-py, 2-CH₃, 5-Cl], [739, single bond, 3-CF₃-2-py, 2-CH₃, 5-Cl], [740, single bond, 2-py, 2-CH₃, 5-F], [741, single bond, 3-CF₃-2-py, 2-CH₃, 5-F], [742, single bond, 2-py, 2-cyclopropyl, 5-CH₃], [743, single bond, 3-CF₃-2-py, 2-cyclopropyl, 5-CH₃], [744, single bond, 6-CF₃-2-py, 2-CH₃, 5-CH₃], [745, single bond, 6-CF₃-2-py, 2-CF₃, 5-CH₃], [746, single bond, 6-CF₃-2-py, 2-CH₃, 5-Cl], [747, single bond, 6-CF₃-2-py, 2-CH₃, 5-F], [748, single bond, 6-CF₃-2-py, 2-cyclopropyl, 5-CH₃], [749, single bond, 3-py, 2-CH₃, 5-CH₃], [750, single bond, 3-py, 2-CF₃, 5-CH₃], [751, single bond, 3-py, 2-CH₃, 5-Cl], [752, single bond, 3-py, 2-CH₃, 5-F].
[753, single bond, 3-py, 2-cyclopropyl, 5-CH₃], [754, single bond, 2-CF₃-3-py, 2-CH₃, 5-CH₃], [755, single bond, 2-CF₃-3-py, 2-CF₃, 5-CH₃], [756, single bond, 2-CF₃-3-py, 2-CH₃, 5-Cl], [757, single bond, 2-CF₃-3-py, 2-CH₃, 5-F], [758, single bond, 2-CF₃-3-py, 2-cyclopropyl, 5-CH₃], [759, single bond, 6-CF₃-3-py, 2-CH₃, 5-CH₃], [760, single bond, 6-CF₃-3-py, 2-CF₃, 5-CH₃], [761, single bond, 6-CF₃-3-py, 2-CH₃, 5-Cl], [762, single bond, 6-CF₃-3-py, 2-CH₃, 5-F], [763, single bond, 6-CF₃-3-py, 2-cyclopropyl, 5-CH₃][764, single bond, 4-py, 2-CH₃, 5-CH₃], [765, single bond, 4-py, 2-CF₃, 5-CH₃], [766, single bond, 4-py, 2-CH₃, 5-Cl], [767, single bond, 4-py, 2-CH₃, 5-F], [768, single bond, 4-py, 2-cyclopropyl, 5-CH₃], [769, single bond, 2-CF₃-4-py, 2-CH₃, 5-CH₃], [770, single bond, 2-CF₃-4-py, 2-CF₃, 5-CH₃], [771, single bond, 2-CF₃-4-py, 2-CH₃, 5-Cl], [772, single bond, 2-CF₃-4-py, 2-CH₃, 5-F], [773, single bond, 2-CF₃-4-py, 2-cyclopropyl, 5-CH₃].

Pests against which the present compound has an activity include, for example, noxious arthropods such as noxious insects and noxious acarines, and nematodes. Specific examples of these pests include the following.

Hemiptera:

Planthoppers (Delphacidae) such as small brown planthopper (Laodelphax striatellus), brown rice planthopper (Nilaparvata lugens), and white-backed rice planthopper (Sogatella furcifera); leafhoppers (Deltocephalidae) such as green rice leafhopper (Nephotettix cincticeps), green rice leafhopper (Nephotettix virescens), and tea green leafhopper (Empoasca onukii); aphids (Aphididae) such as cotton aphid (Aphis gossypii), green peach aphid (Myzus persicae), cabbage aphid (Brevicoryne brassicae), piraea aphid (Aphis spiraecola), potato aphid (Macrosiphum euphorbiae), foxglove aphid (Aulacorthum solani), oat bird-cherry aphid (Rhopalosiphum padi), tropical citrus aphid (Toxoptera citricidus), and mealy plum aphid (Hyalopterus pruni); stink bugs (Pentatomidae) such as green stink bug (Nezara antennata), bean bug (Riptortus clavetus), rice bug (Leptocorisa chinensis), white spotted spined bug (Eysarcoris parvus), and stink bug (Halyomorpha mista); whiteflies (Aleyrodidae) such as greenhouse whitefly (Trialeurodes vaporariorum), sweetpotato whitefly (Bemisia tabaci), citrus whitefly (Dialeurodes citri), and citrus spiny white fly (Aleurocanthus spiniferus); scales (Coccidae) such as Calfornia red scale (Aonidiella aurantii), San Jose scale (Comstockaspis pernicios), citrus north scale (Unaspis citri), red wax scale (Ceroplastes rubens), cottonycushion scale (Icerya purchasi), Japanese mealybug (Planococcus kraunhiae), Cosmstock mealybug (Pseudococcus longispinis), and white peach scale (Pseudaulacaspis pentagona); lace bugs (Tingidae); cimices such as Cimex lectularius; psyllids (Psyllidae).

Lepidoptera:

Pyralid moths (Pyralidae) such as rice stem borer (Chile suppressalis), yellow rice borer (Tryporyza incertulas), rice leafroller (Cnaphalocrocis medinalis), cotton leafroller (Notarcha derogata), Indian meal moth (Plodia interpunctella), oriental corn borer (Ostrinia furnacalis), cabbage webworm (Hellula undalis), and bluegrass webworm (Pediasia teterrellus); owlet moths (Noctuidae) such as common cutworm (Spodoptera litura), beet armyworm (Spodoptera exigua), armyworm (Pseudaletia separata), cabbage armyworm (Mamestra brassicae), black cutworm (Agrotis ipsilon), beet semi-looper (Plusia nigrisigna), Thoricoplusia spp., Heliothis spp., and Helicoverpa spp.; white butterflies (Pieridae) such as common white (Pieris rapae); tortricid moths (Tortricidae) such as Adoxophyes spp., oriental fruit moth (Grapholita molesta), soybean pod borer (Leguminivora glycinivorella), azuki bean podworm (Matsumuraeses azukivora), summer fruit tortrix (Adoxophyes orana fasciata), smaller tea tortrix (Adoxophyes honmai), oriental tea tortrix (Homona magnanima), apple tortrix (Archips fuscocupreanus), and codling moth (Cydia pomonella); leafblotch miners (Gracillariidae) such as tea leafroller (Caloptilia theivora), and apple leafminer (Phyllonorycter ringoneella); Carposinidae such as peach fruit moth (Carposina niponensis); lyonetiid moths (Lyonetiidae) such as Lyonetia spp.; tussock moths (Lymantriidae) such as Lymantria spp., and Euproctis spp.; yponomeutid moths (Yponomeutidae) such as diamondback (Plutella xylostella); gelechiid moths (Gelechiidae) such as pink bollworm (Pectinophora gossypiella), and potato tubeworm (Phthorimaea operculella); tiger moths and allies (Arctiidae) such as fall webworm (Hyphantria cunea); tineid moths (Tineidae) such as casemaking clothes moth (Tinea translucens), and webbing clothes moth (Tineola bisselliella).

Thysanoptera:

Thrips (Thripidae) such as yellow citrus thrips (Frankliniella occidentalis), melon thrips (Thrips palmi), yellow tea thrips (Scirtothrips dorsalis), onion thrips (Thrips tabaci), flower thrips (Frankliniella intonsa).

Diptera:

Culices such as common mosquito (Culex pipiens pallens), Cluex tritaeniorhynchus, and Cluex quinquefasciatus; Aedes spp. such as yellow fever mosquito (Aedes aegypti), and Asian tiger mosquito (Aedes albopictus); Anopheles spp. such as Anopheles sinensis; chironomids (Chironomidae); house flies (Muscidae) such as Musca domestica, and Muscina stabulans; blow flies (Calliphoridae); flesh flies (Sarcophagidae); little house flies (Fanniidae); anthomyiid flies (Anthomyiidae) such as seedcorn fly (Delia platura), and onion fly (Delia antique); leafminer flies (Agromyzidae) such as rice leafminer (Agromyza oryzae), little rice leafminer (Hydrellia griseola), tomato leafminer (Liriomyza sativae), legume leafminer (Liriomyza trifolii), and garden pea leafminer (Chromatomyia horticola); gout flies (Chloropidae) such as rice stem maggot (Chlorops oryzae); fruit flies (Tephritidae) such as melon fly (Dacus cucurbitae), and Meditteranean fruit fly (Ceratitis capitata); Drosophilidae; humpbacked flies (Phoridae) such as Megaselia spiracularis; moth flies (Psychodidae) such as Clogmia albipunctata; Simuliidae; Tabanidae such as horsefly (Tabanus trigonus); stable flies.

Coleoptera:

Corn root worms (Diabrotica spp.) such as Western corn root worm (Diabrotica virgifera virgifera), and Sourthern corn root worm (Diabrotica undecimpunctata howardi); scarabs (Scarabaeidae) such as cupreous chafer (Anomala cuprea), soybean beetle (Anomala rufocuprea), and Japanese beetle (Popillia japonica); weevils such as maize weevil (Sitophilus zeamais), rice water weevil (Lissorhoptrus oryzophilus), azuki bean weevil (Callosobruchus chinensis), rice curculio (Echinocnemus squameus), boll weevil (Anthonomus grandis), and hunting billbug (Sphenophorus venatus); darkling beetles (Tenebrionidae) such as yellow mealworm (Tenebrio molitor), and red flour beetle (Tribolium castaneum); leaf beetles (Chrysomelidae) such as rice leaf beetle (Oulema oryzae), cucurbit leaf beetle (Aulacophora femoralis), striped flea beetle (Phyllotreta striolata), and Colorado potato beetle (Leptinotarsa decemlineata); dermestid beetles (Dermestidae) such as varied carper beetle (Anthrenus verbasci), and hide beetle (Dermestes maculates); deathwatch beetles (Anobiidae) such as cigarette beetle (Lasioderma serricorne); Epilachna such as Twenty-eight-spotted ladybird (Epilachna vigintioctopunctata); bark beetles (Scolytidae) such as powder-post beetle (Lyctus brunneus), and pine shoot beetle (Tomicus piniperda); false powder-post beetles (Bostrychidae); spider beetles (Ptinidae); longhorn beetles (Cerambycidae) such as white-spotted longicorn beetle (Anoplophora malasiaca); click beetles (Agriotes spp.); Paederus fuscipens.

Orthoptera:

Asiatic locust (Locusta migratoria), African mole cricket (Gryllotalpa africana), rice grasshopper (Oxya yezoensis), rice grasshopper (Oxya japonica), Gryllidae.

Hymenoptera:

Ants (Formicidae) such as pharaoh ant (Monomorium pharaosis), negro ant (Formica fusca japonica), black house ant (Ochetellus glaber), Pristomyrmex pungens, Pheidole noda, leaf-cutting ant (Acromyrmex spp.), and fire ant (Solenopsis spp.); hornets (Vespidae); bethylid wasps (Betylidae); sawflies (Tenthredinidae) such as cabbage sawfly (Athalia rosae), and Athalia japonica.

Nematoda:

Aphelenchoides besseyi, Nothotylenchus acris, Meloidogyne incognita, Meloidogyne hapla, Meloidogyne javanica, Heterodera glycines, Globodera rostochiensis, Pratylenchus coffeae, Pratylenchus neglectus.

Blattodea:

German cockroach (Blattella germanica), smokybrown cockroach (Periplaneta fuliginosa), American cockroach (Periplaneta americana), Periplaneta brunnea, oriental cockroach (Blatta orientalis);

Acarina:

Spider mites (Tetranychidae) such as two-spotted spider mite (Tetranychus urticae), Kanzawa spider mite (Tetranychus kanzawai), citrus red mite (Panonychus citri), European red mite (Panonychus ulmi), and Oligonychus spp.; eriophyid mites (Eriophyidae) such as pink citrus rust mite (Aculops pelekassi), Phyllocoptruta citri, tomato rust mite (Aculops lycopersici), purple tea mite (Calacarus carinatus), pink tea rust mite (Acaphylla theavagran), Eriophyes chibaensis, and apple rust mite (Aculus schlechtendali); tarosonemid mites (Tarsonemidae) such as broad mite (Polyphagotarsonemus latus); false spider mites (Tenuipalpidae) such as Brevipalpus phoenicis; Tuckerellidae; ticks (Ixodidae) such as Haemaphysalis longicornis, Haemaphysalis flava, Dermacentor taiwanicus, Ixodes ovatus, Ixodes persulcatus, black legged tick (Ixodes scapularis), Boophilus microplus, and Rhipicephalus sanguineus; acarid mites (Acaridae) such as mold mite (Tyrophagus putrescentiae), and Tyrophagus similis; house dust mites (Pyroglyphidae) such as Dermatophagoides farinae, and Dermatophagoides ptrenyssnus; cheyletide mites (Cheyletidae) such as Cheyletus eruditus, Cheyletus malaccensis, and Cheyletus moorei; parasitoid mites (Dermanyssidae) such as tropical rat mite (Ornithonyssus bacoti), northern fowl mite (Ornithonyssus sylviarum), and poultry red mite (Dermanyssus gallinae); chiggers (Trombiculidae) such as Leptotrombidium akamushi; spiders (Araneae) such as Japanese foliage spider (Chiracanthium japonicum), redback spider (Latrodectus hasseltii).

The pest controlling agent of the present invention contains the present compound and an inert carrier. Generally, the pest controlling agent of the present invention is a formulation obtained by mixing the present compound and an inert carrier such as a solid carrier, a liquid carrier and a gaseous carrier, and further adding a surfactant and other adjuvant for formulation, if necessary. The formulation includes, for example, an emulsion, an oil solution, a powder, a granule, a wettable powder, a flowable formulation, a microcapsule, an aerosol, a smoking agent, a poison bait, and a resin formulation. In the pest controlling agent of the present invention, the present compound is usually contained in an amount of 0.01% to 95% by weight.

The solid carrier used for formulation includes, for example, a fine power and a granule of clays (e.g., kaolin clay, diatomite, bentonite, Fubasami clay, and acid clay), synthetic hydrated silicon oxide, talc, ceramic, other inorganic minerals (e.g., sericite, quartz, sulfur, activated carbon, calcium carbonate, hydrated silica) or chemical fertilizers (e.g., ammonium sulfate, ammonium phosphate, ammonium nitrate, urea, and ammonium chloride).

The liquid carrier includes, for example, water, alcohols (e.g., methanol, ethanol, 2-propanol, butanol, hexanol, benzyl alcohol, ethylene glycol, propylene glycol, phenoxyethanol), ketones (e.g., acetone, methyl ethyl ketone, cyclohexanone), aromatic hydrocarbons (e.g., toluene, xylene, ethylbenzene, dodecylbenzene, phenylxylylethane, methylnaphthalene), aliphatic hydrocarbons (e.g., hexane, cyclohexane, kerosine, light oil), esters (e.g., ethyl acetate, butyl acetate, isopropyl mylistate, ethyl oleate, diisopropyl adipate, diisobutyl adipate, propyleneglycol monomethyl ether acetate), nitriles (e.g., acetonitrile, isobutyronitrile), ethers (e.g., diisopropyl ether, 1,4-dioxane, ethylene glycol dimethyl ether, diethylene glycol dimethyl ether, diethylene glycol monomethyl ether, propylene glycol monomethyl ether, dipropylene glycol monomethyl ether, 3-methoxy-3-methyl-1-butanol), acid amides (e.g., N,N-dimethylformamide, N,N-dimethylacetamide), halogenated hydrocarbons (e.g., dichloromethane, trichloroethane, tetrachlorocarbon), sulfoxides (e.g., dimethylsulfoxide), propylene carbonate, and vegetable oils (e.g., soy bean oil, cotton seed oil).

The gaseous carrier includes, for example, fluorocarbons, butane gas, liquefied petroleum gas (LPG), dimethyl ether, and carbon dioxide.

The surfactant includes, for example, nonionic surfactant, such as polyoxyethylene alkyl ether, polyoxyethylene alkylaryl ether, polyethyleneglycol fatty acid ester; and anionic surfactant, such as alkylsulfonic acid salts, alkylbenzenesulfonic acid salts, and alkylsurfic acid salts.

The other adjuvant for formulation includes, for example, binders, dispersants, colorants and stabilizers, and specifically for example, casein, gelatin, polysaccharides (e.g., starch, gum arabic, cellulose derivatives, alginic acid), lignin derivatives, synthetic water-soluble polymers (e.g., polyvinyl alcohol, polyvinylpyrrolidone, polyacrylic acid), PAP (isopropyl acid phosphate), BHT (2,6-di-t-butyl-4-methylphenol), BHA (a mixture of 2-t-butyl-4-methoxyphenol and 3-t-butyl-4-methoxyphenol).

The method for controlling pests of the present invention is applying an effective amount of the present compound to pests directly and/or habitats of pests (e.g., plant, soil, indoor, and in-body of animals). The present compound is usually used as the pest controlling agent of the present invention for the method for controlling pests of the present invention.

When the pest controlling agent of the present invention is used for a control of pests in agriculture, the application amount is usually 1 to 10,000 g as the present compound per 10,000 m². When the pest controlling agent of the present invention is a formulation of emulsions, wettable powders or flowables, they are usually applied after a dilution with water to have an active ingredient concentration of 0.01 to 10000 ppm. When the pest controlling agent of the present invention is a formulation of granules or powders, they are usually applied as such.

These formulations and the dilute aqueous solution of the formulation may be sprayed directly to the plant to be protected from pests, and may be applied to the soil to control the pests living in a soil.

Furthermore, the resin formulations of sheets or strip form can be applied by a method such as winding around plants, stretching in the vicinity of plants and laying on the soil surface at the plant bottom.

When a pest controlling agent according to the present invention is used to control pests (for example, fly, mosquito, cockroach) which inhabit in the house, the application amount is usually from 0.01 to 1,000 mg in terms of the amount of the present compound per 1 m² of an area to be treated in the case of treating on a surface, while the application amount is usually from 0.01 to 500 mg in terms of the amount of the present compound per 1 m² of an area to be treated in the case of treating on a space. When the pest controlling agent of the present invention is formulated into emulsifiable concentrates, wettable powders and flowable preparations, they are usually applied after dilution so as to adjust the concentration of an active ingredient within a range from 0.1 to 1,000 ppm, while oil preparations, aerosol preparations, fumigants and poison baits are applied without dilution.

The pest controlling agent of the present invention could be used in farmlands on which “crops” shown below are cultivated.

### “Crops”

Agricultural crops: corn, rice, wheat, barley, rye, oat, sorghum, cotton, soybean, peanut, sarrazin, sugar beet, rapeseed, sunflower, sugar cane, tobacco;

Vegetables: Solanaceae vegetables (eggplant, tomato, green pepper, hot pepper, and potato), Cucurbitaceae vegetables (cucumber, pumpkin, zucchini, watermelon, and melon), Cruciferae vegetables (Japanese radish, turnip, horseradish, kohlrabi, Chinese cabbage, cabbage, brown mustard, broccoli, and cauliflower), Compositae vegetables (burdock, garland chrysanthemum, artichoke, and lettuce), Liliaceae vegetables (Welsh onion, onion, garlic, and asparagus), Umbelliferae vegetables (carrot, parsley, celery, and parsnip), Chenopodiaceae vegetables (spinach, and Swiss chard), Labiatae vegetables (Japanese basil, mint, and basil), strawberry, sweat potato, yam, aroid;

Fruit trees: pomaceous fruits (apple, common pear, Japanese pear, Chinese quince, and quince), stone fleshy fruits (peach, plum, nectarine, Japanese plum, cherry, apricot, and prune), citrus plants (Satsuma mandarin, orange, lemon, lime, and grapefruit), nuts (chestnut, walnut, hazel nut, almond, pistachio, cashew nut, and macadamia nut), berry fruits (blueberry, cranberry, blackberry, and raspberry), grape, persimmon, olive, loquat, banana, coffee, date, coconut palm, and oil palm;

Trees other fruit trees: tea, mulberry, flowering trees (azalea, japonica, hydrangea, sasanqua, Illicium anisatum, cherry tree, tulip poplar, crepe myetle, and orange osmanthus), street trees (ash tree, birch, dogwood, eucalyptus, ginkgo, lilac, maple tree, oak, poplar, cercis, Chinese sweet gum, plane tree, zelkova, Japanese arborvitae, fir tree, Japanese hemlock, needle juniper, pine, spruce, yew, elm, and horse-chestnut), sweet viburnum, Podocarpus macrophyllus, Japanese cedar, Japanese cypress, croton, spindle tree, Chainese hawthorn.

Lawn: zoysia (Japanese lawn grass, mascarene grass), Bermuda grass (Cynodon dactylon), bent grass (creeping bent grass, Agrostis stolonifera, Agrostis tenuis), bluegrass (Kentucky bluegrass, rough bluegrass), fescue (tall fescue, chewing fescue, creeping fescue), ryegrass (darnel, perennial ryegrass), cocksfoot, timothy grass;

Others: flowers (rose, carnation, chrysanthemum, Eustoma grandiflorum Shinners (prairie gentian), gypsophila, gerbera, pot marigold, salvia, petunia, verbena, tulip, aster, gentian, lily, pansy, cyclamen, orchid, lily of the valley, lavender, stock, ornamental kale, primula, poinsttia, gladiolus, cattleya, daisy, verbena, cymbidium, begonia), biofuel plants (Jatropha, curcas, safflower, Camelina alyssum, switchgrass, miscanthus, reed canary grass, Arundo donax, kenaf, cassava, willow, algae), foliage plant.

The “crops” include genetically modified crops.

The pest controlling agents of the present invention can be a admixture with or together with other insecticides, acaricides, nematocides, fungicides, plant growth regulators, herbicides, and synergists.

Examples of active ingredients of the insecticide, the acaricide, the nematocide, the fungicide, the plant growth regulator, the herbicide, and the synergist are shown below.

Active ingredients of the insecticides:

### (1) Organic Phosphorus Compounds:

Acephate, Aluminium phosphide, butathiofos, cadusafos, chlorethoxyfos, chlorfenvinphos, chlorpyrifos, chlorpyrifos-methyl, cyanophos: CYAP, diazinon, DCIP (dichlorodiisopropyl ether), dichlofenthion: ECP, dichlorvos (DDVP), dimethoate, dimethylvinphos, disulfoton, EPN, ethion, ethoprophos, etrimfos, fenthion: MPP, fenitrothion: MEP, fosthiazate, formothion, hydrogen phosphide, isofenphos, isoxathion, malathion, mesulfenfos, methidathion: DMTP, monocrotophos, naled: BRP, oxydeprofos: ESP, parathion, phosalone, phosmet: PMP, pirimiphos-methyl, pyridafenthion, guinalphos, phenthoate: PAP, profenofos, propaphos, prothiofos, pyraclorfos, salithion, sulprofos, tebupirimfos, temephos, tetrachlorvinphos, terbufos, thiometon, trichlorphon: DEP, vamidothion, phorate, cadusafos;

### (2) Carbamate Compounds:

Alanycarb, bendiocarb, benfuracarb, BPMC, carbaryl, carbofuran, carbosulfan, cloethocarb, ethiofencarb, fenobucarb, fenothiocarb, fenoxycarb, furathiocarb, isoprocarb: MIPC, metolcarb, methomyl, methiocarb, NAC, oxamyl, pirimicarb, propoxur: PHC, XMC, thiodicarb, xylylcarb, aldicarb;

### (3) Pyrethroid Compounds:

Acrinathrin, allethrin, benfluthrin, beta-cyfluthrin, bifenthrin, cycloprothrin, cyfluthrin, cyhalothrin, cypermethrin, deltamethrin, esfenvalerate, ethofenprox, fenpropathrin, fenvalerate, flucythrinate, flufenoprox, flumethrin, fluvalinate, halfenprox, imiprothrin, permethrin, prallethrin, pyrethrins, resmethrin, sigma-cypermethrin, silafluofen, tefluthrin, tralomethrin, transfluthrin, tetramethrin, phenothrin, cyphenothrin, alpha-cypermethrin, zeta-cypermethrin, lambda-cyhalothrin, gamma-cyhalothrin, furamethrin, tau-fluvalinate, metofluthrin, profluthrin, dimefluthrin, 2,3,5,6-tetrafluoro-4-(methoxymethyl)benzyl (EZ)-(1RS,3RS;1RS,3SR)-2,2-dimethyl-3-prop-1-enylcyclopropanecarboxylate, 2,3,5,6-tetrafluoro-4-methylbenzyl (EZ)-(1RS,3RS;1RS,3SR)-2,2-dimethyl-3-prop-1-enylcyclopropanecarboxylate, and 2,3,5,6-tetrafluoro-4-(methoxymethyl)benzyl(1RS,3RS;1RS,3SR)-2,2-dimethyl-3-(2-methyl-1-propenyl)cyclopropanecarboxylate;

### (4) Nereistoxin Compounds:

Cartap, bensultap, thiocyclam, monosultap, bisultap;

### (5) Neonicotinoid Compounds:

Imidacloprid, nitenpyram, acetamiprid, thiamethoxam, thiacloprid, dinotefuran, clothianidin;

### (6) Benzoylurea Compounds:

Chlorfluazuron, bistrifluron, diafenthiuron, diflubenzuron, fluazuron, flucycloxuron, flufenoxuron, hexaflumuron, lufenuron, novaluron, noviflumuron, teflubenzuron, triflumuron, triazuron;

### (7) Phenylpyrazole Compounds:

Acetoprole, ethiprole, fipronil, vaniliprole, pyriprole, pyrafluprole;

### (8) Bt Toxins:

Live spores derived from and crystal toxins produced from Bacillus thuringiesis and a mixture thereof;

### (9) Hydrazine Compounds:

Chromafenozide, halofenozide, methoxyfenozide, tebufenozide;

### (10) Organic Chlorine Compounds:

Aldrin, dieldrin, dienochlor, endosulfan, methoxychlor;

### (11) Other Insecticidal Active Ingredients:

Machine oil, nicotine-sulfate; avermectin-B, bromopropylate, buprofezin, chiorphenapyr, cyantraniliprole, cyromazine, D-D(1,3-Dichloropropene, emamectin-benzoate, fenazaquin, flupyrazofos, hydroprene, methoprene, indoxacarb, metoxadiazone, milbemycin-A, pymetrozine, pyridalyl, pyriproxyfen, spinosad, sulfluramid, tolfenpyrad, triazamate, flubendiamide, lepimectin, Arsenic acid, benclothiaz, Calcium cyanamide, Calcium polysulfide, chlordane, DDT, DSP, flufenerim, flonicamid, flurimfen, formetanate, metam-ammonium, metam-sodium, Methyl bromide, Potassium oleate, protrifenbute, spiromesifen, sulfoxaflor, Sulfur, metaflumizone, spirotetramat, pyrifluquinazone, spinetoram, chlorantraniliprole, tralopyril, cyantraniliprole, any compound represented by the following formula (K):

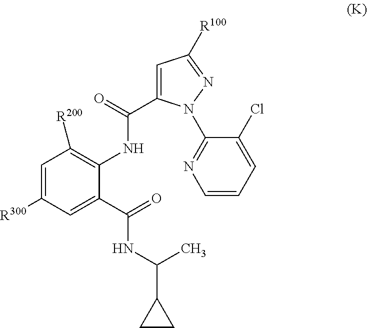

wherein
R¹⁰⁰ represents chlorine, bromine or a trifluoromethyl group,
R²⁰⁰ represents chlorine, bromine or a methyl group,
R³⁰⁰ represents chlorine, bromine or a cyano group
and, any compound represented by the following formula (L):

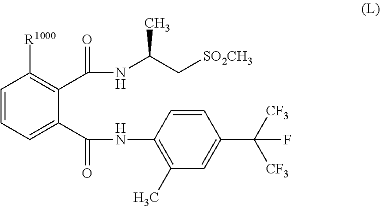

wherein
R¹⁰⁰⁰ represents chlorine, bromine or iodide.
   Active ingredients of the Acardides:

Acequinocyl, amitraz, benzoximate, bifenaate, bromopropylate, chinomethionat, chlorobenzilate, CPCBS(chlorfenson), clofentezine, cyflumetofen, dicofol, etoxazole, fenbutatin oxide, fenothiocarb, fenpyroximate, fluacrypyrim, fluproxyfen, hexythiazox, propargite: BPPS, polynactins, pyridaben, Pyrimidifen, tebufenpyrad, tetradifon, spirodiclofen, spiromesifen, spirotetramat, amidoflumet, cyenopyrafen.

Active Ingredients of the Nematocides:

DCIP, fosthiazate, levamisol, methyisothiocyanate, morantel tartarate, imicyafos.

Active Ingredients of the Fungicides:

Azole fungicidal compounds such as propiconazole, prothioconazole, triadimenol, prochloraz, penconazole, tebuconazole, flusilazole, diniconazole, bromuconazole, epoxiconazole, difenoconazole, cyproconazole, metconazole, triflumizole, tetraconazole, myclobutanil, fenbuconazole, hexaconazole, fluquinconazole, triticonazole, bitertanol, imazalil, and flutriafol;

Cyclic amine fungicidal compouds such as fenpropimorph, tridemorph, and fenpropidin;

Benzimidazole fungicidal compounds such as carbendezim, benomyl, thiabendazole, and thiophanate-methyl;

Procymidone; cyprodinil; pyrimethanil; diethofencarb; thiuram; fluazinam; mancozeb; iprodione; vinclozolin; chlorothalonil; captan; mepanipyrim; fenpiclonil; fludioxonil; dichlofluanid; folpet; kresoxim-methyl; azoxystrobin; trifloxystrobin; fluoxastrobin; picoxystrobin; pyraclostrobin; dimoxystrobin; pyribencarb; spiroxamine; quinoxyfen; fenhexamid; famoxadone; fenamidone; zoxamide; ethaboxam; amisulbrom; iprovalicarb; benthiavalicarb); cyazofamid; mandipropamid; boscalid; penthiopyrad; metrafenone; fluopiran; bixafen; cyflufenamid; proquinazid; isotianil, tiadinil.

Active Ingredients of the Herbicides:

### (1) Phenoxyfatty Acid Herbicidal Compounds

2,4-PA, MCP, MCPB, phenothiol, mecoprop, fluroxypyr, triclopyr, clomeprop, and naproanilide.

### (2) Benzoic Acid Herbicidal Compounds

2,3,6-TBA, dicamba, clopyralid, picloram, aminopyralid, quinclorac, and quinmerac.

### (3) Urea Herbicidal Compounds

diuron, linuron, chlortoluron, isoproturon, fluometuron, isouron, tebuthiuron, methabenzthiazuron, cumyluron, daimuron, and methyl-daimuron.

### (4) Triazine Herbicidal Compounds

atrazine, ametoryn, cyanazine, simazine, propazine, simetryn, dimethametryn, prometryn, metribuzin, indaziflam, and triaziflam.

### (5) Bipyridinium Herbicidal Compounds

paraquat, and diquat.

### (6) Hydroxybenzonitrile Herbicidal Compounds

bromoxynil and ioxynil.

### (7) Dinitroaniline Herbicidal Compounds

pendimethalin, prodiamine, and trifluralin.

### (8) Organic Phosphorus Herbicidal Compounds

amiprofos-methyl, butamifos, bensulide, piperophos, anilofos, glyphosate, glufosinate, and bialaphos.

### (9) Carbamate Herbicidal Compounds

di-allate, tri-allate, EPTC, butylate, benthiocarb, esprocarb, molinate, dimepiperate, swep, chlorpropham, phenmedipham, phenisopham, pyributicarb, and asulam;

### (10) Acid Amide Herbicidal Compounds

propanil, propyzamide, bromobutide, and etobenzanid.

### (11) Chloroacetanilide Herbicidal Compounds

acetochlor, alachlor, butachlor, dimethenamid, propachlor, metazachlor, metolachlor, pretilachlor, thenylchlor, and pethoxamid.

### (12) Diphenylether Herbicidal Compounds

acifluorfen-sodium, bifenox, oxyfluorfen, lactofen, fomesafen, chlomethoxynil, and aclonifen.

### (13) Cyclic Imide Herbicidal Compounds

oxadiazon, cinidon-ethyl, carfentrazone-ethyl, surfentrazone, flumiclorac-pentyl, flumioxazin, pyraflufen-ethyl, oxadiargyl, pentoxazone, fluthiacet-methyl, butafenacil, benzfendizone, and saflufenacil.

### (14) Pyrazole Herbicidal Compounds

benzofenap, pyrazolate, pyrazoxyfen, topramezone, and pyrasulfotole.

### (15) Triketone Herbicidal Compounds

isoxaflutole, benzobicyclon, sulcotrione, mesotrione, tembotrione, and tefuryltrione.

### (16) Aryloxyphenoxypropionic Acid Herbicidal Compounds

clodinafop-propargyl, cyhalofop-butyl, diclofop-methyl, fenoxaprop-ethyl, fluazifop-butyl, haloxyfop-methyl, and quizalofop-ethyl and metamifol.

### (17) Trioneoxime Herbicidal Compounds

alloxydim-sodium, sethoxydim, butroxydim, clethodim, cloproxydim, cycloxydim, tepraloxydim, tralkoxydim, and profoxydim.

### (18) Sulfonylurea Herbicidal Compounds

chlorsulfuron, sulfometuron-methyl, metsulfuron-methyl, chlorimuron-ethyl, tribenuron-methyl, triasulfuron, bensulfuron-methyl, thifensulfuron-methyl, pyrazosulfuron-ethyl, primisulfuron-methyl, nicosulfuron, amidosulfuron, cinosulfuron, imazosulfuron, rimsulfuron, halosulfuron-methyl, prosulfuron, ethametsulfuron-methyl, triflusulfuron-methyl, flazasulfuron, cyclosulfamuron, flupyrsulfuron, sulfosulfuron, azimsulfuron, ethoxysulfuron, oxasulfuron, iodosulfuron-methyl-sodium, foramsulfuron, mesosulfuron-methyl, trifloxysulfuron, tritosulfuron, orthosulfamuron, flucetosulfuron, and propyrisulfuron.

### (19) Imidazolinone Herbicidal Compounds

imazamethabenz-methyl, imazamethapyr, imazamox, imazapyr, imazaquin, and imazethapyr.

### (20) Sulfonamide Herbicidal Compounds

flumetsulam, metosulam, diclosulam, florasulam, cloransulam-methyl, penoxsulam, and pyroxsulam.

### (21) Pyrimidinyloxybenzoic Acid Herbicidal Compounds

pyrithiobac-sodium, bispyribac-sodium, pyriminobac-methyl, pyribenzoxim, pyriftalid, and pyrimisulfan.

### (22) Other Herbicidal Compounds

Bentazon, bromacil, terbacil, chlorthiamid, isoxaben, dinoseb, amitrole, cinmethylin, tridiphane, dalapon, diflufenzopyr-sodium, dithiopyr, thiazopyr, flucarbazone-sodium, propoxycarbazone-sodium, mefenacet, flufenacet, fentrazamide, cafenstrole, indanofan, oxaziclomefone, benfuresate, ACN, pyridate, chloridazon, norflurazon, flurtamone, diflufenican, picolinafen, beflubutamid, clomazone, amicarbazone, pinoxaden, pyraclonil, pyroxasulfone, thiencarbazone-methyl, aminocyclopyrachlor, ipfencarbazone, and methiozolin.

Active Ingredients of the Synergists:

Piperonyl butoxide, sesamex, sulfoxide, N-(2-ethylhexyl)-8,9,10-trinorborn-5-ene-2,3-dicarboxyimide (MGK 264), N-declyimidazole, WARF-antiresistant, TBPT, TPP, IBP, PSCP, methyl iodide (CH3I), t-phenylbutenone, diethylmaleate, DMC, FDMC, ETP, and ETN.

### EXAMPLES

The present invention will be described in more detail below by way of Production Examples, Formulation Examples and Test Examples, but the present invention is not limited to these Examples.

In Production Examples and Reference Production Examples, ¹H-NMR data is shown as a data which is measured by using tetramethylsilane as an internal standard in a deutero chloroform solvent, unless otherwise stated.

First, Production Examples of the present compound are shown below.

### Production Example 1

To 14 ml of tetrahydrofuran, 1.5 g of 6-trifluoromethylpyrimidine-4-carboxamide oxime and 1.7 g of 1,1′-carbonyldiimidazole were added. This mixture was stirred at room temperature for 1 hour and a half. To the mixture, 1.2 g of 1,8-diazabicyclo[5.4.0]undec-7-ene was added, followed by stirring at room temperature for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1.4 g of 3-(6-trifluoromethylpyrimidin-4-yl)-1,2,4-oxadiazol-5-one (hereinafter referred to as the present compound (1)).

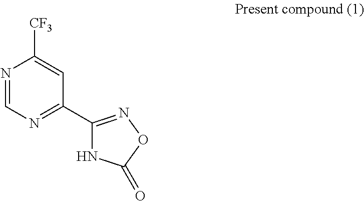

¹H-NMR (DMSO-d₆): 8.41(s,1H), 9.69(s,1H)

### Production Example 2

To a mixture of 2 ml of pyridine, 0.23 g of 1,8-diazabicyclo[5.4.0]undec-7-ene and 0.25 g of 3-(6-trifluoromethylpyrimidin-4-yl)-1,2,4-oxadiazol-5-one, 0.2 g of 1-pyrrolidinecarbonyl chloride was added at room temperature. This mixture was stirred at 50° C. for 10 hours. The reaction mixture was left stand to cool to room temperature, and then concentrated. The residue was subjected to silica gel column chromatography to obtain 0.08 g of 4-(1-pyrrolidinecarbonyl)-3-(6-trifluoromethylpyrimidin-4-yl)-1,2,4-oxadiazol-5-one (hereinafter referred to as the present compound (2)).

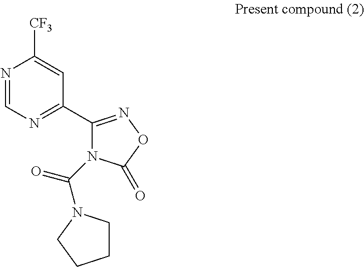

¹H-NMR: 2.06-2.10(m,4H), 3.63-3.73(m,4H), 8.24(s,1H), 9.45(s,1H)

### Production Example 3

To 18 ml of tetrahydrofuran, 2.4 g of 2-(1,1-dimethylethyl)-6-trifluoromethylpyrimidine-4-carboxamide oxime and 2.1 g of 1,1′-carbonyldiimidazole were added. This mixture was stirred at room temperature for 1 hour and a half. To this mixture, 1.95 g of 1,8-diazabicyclo[5.4.0]undec-7-ene was added, followed by stirring at room temperature for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1.9 g of 3-[2-(1,1-dimethylethyl)-6-trifluoromethylpyrimidin-4-yl]-1,2,4-oxadiazol-5-one (hereinafter referred to as the present compound (3)).

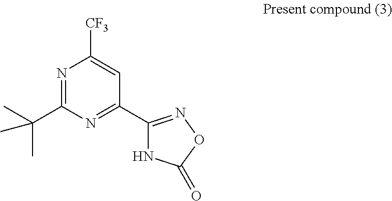

¹H-NMR: 1.49(s,9H), 8.08(s,1H)

### Production Example 4

To a mixture of 2 ml of pyridine, 0.22 g of 1,8-diazabicyclo[5.4.0]undec-7-ene and 0.3 g of 3-[2-(1,1-dimethylethyl)-6-trifluoromethylpyrimidin-4-yl]-1,2,4-oxadiazol-5-one, 0.2 g of 1-pyrrolidinecarbonyl chloride was added at room temperature. This mixture was stirred at 50° C. for 6 hours. The reaction mixture was cooled to room temperature and then concentrated. The residue was subjected to silica gel column chromatography to obtain 0.15 g of 3-[2-(1,1-dimethylethyl)-6-trifluoromethylpyrimidin-4-yl]-4-(1-pyrrolidinecarbonyl)-1,2,4-oxadiazol-5-one (hereinafter referred to as the present compound (4)).

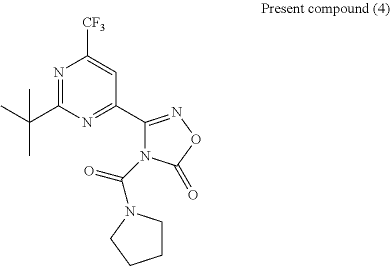

¹H-NMR: 1.42(s,9H), 2.05-2.10(m,4H), 3.64-3.76(m,4H), 8.01(s,1H)

### Production Example 5

To 20 ml of tetrahydrofuran, 2.0 g of 6-difluoromethylpyrimidine-4-carboxamide oxime and 2.4 g of 1,1′-carbonyldiimidazole were added. This mixture was stirred at room temperature for 1 hour and a half. To this mixture, 2.27 g of 1,8-diazabicyclo[5.4.0]undec-7-ene was added, followed by stirring at room temperature for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 2.2 g of 3-(6-difluoromethylpyrimidin-4-yl)-1,2,4-oxadiazol-5-one (hereinafter referred to as the present compound (5)).

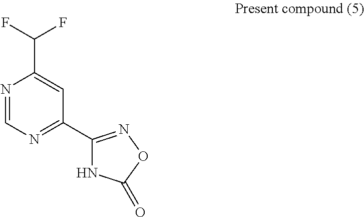

¹H-NMR (DMSO-d₆): 7.14(t,1H), 8.21(s,1H), 9.58(s,1H), 13.59(bs,1H)

### Production Example 6

To a mixture of 2 ml of pyridine, 0.2 g of 1,8-diazabicyclo[5.4.0]undec-7-ene and 0.2 g of 3-(6-difluoromethylpyrimidin-4-yl)-1,2,4-oxadiazol-5-one, 0.18 g of 1-pyrrolidinecarbonyl chloride was added at room temperature. This mixture was stirred at 50° C. for 6 hours. The reaction mixture was left standing to cool to room temperature and then concentrated. The residue was subjected to silica gel column chromatography to obtain 0.12 g of 3-(6-difluoromethylpyrimidin-4-yl)-4-(1-pyrrolidinecarbonyl)-1,2,4-oxadiazol-5-one (hereinafter referred to as the present compound (6)).

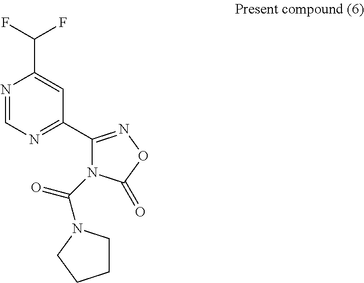

¹H-NMR: 2.05-2.10(m,4H), 3.62-3.73(m,4H), 6.63(t,1H), 8.21(s,1H), 9.36(s,1H)

### Production Example 7

0.05 g of sodium hydride (60% in oil) was suspended in 2 ml of N,N-dimethylformamide, and 0.2 g of 3-(6-trifluoromethylpyrimidin-4-yl)-1,2,4-oxadiazol-5-one was added thereto at room temperature. This mixture was stirred for 10 minutes. To the mixture, 0.18 g of chloromethyl pivalate was added. This mixture was stirred at 80° C. for 4 hours. The reaction mixture was left standing to cool to room temperature. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 0.17 g of 4-[(2,2-dimethyl-1-oxopropoxy)methyl]-3-(6-trifluoromethylpyrimidin-4-yl)-1,2,4-oxadiazol-5-one (hereinafter referred to as the present compound (7)).

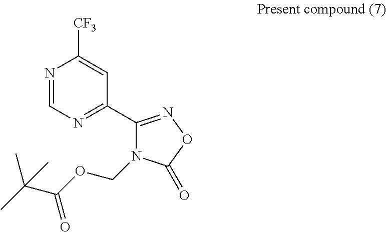

¹H-NMR: 1.12(s,9H), 6.17(s,2H), 8.35(s,1H), 9.52(s,1H)

### Production Example 8

0.05 g of sodium hydride (60% in oil) was suspended in 2 ml of N,N-dimethylformamide, and 0.2 g of 3-(6-difluoromethylpyrimidin-4-yl)-1,2,4-oxadiazol-5-one was added thereto at room temperature. This mixture was stirred at for 10 minutes. To the mixture, 0.2 g of chloromethyl pivalate was added. This mixture was stirred at 80° C. for 4 hours. The reaction mixture was left standing to cool to room temperature. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 0.18 g of 4-[(2,2-dimethyl-1-oxopropoxy)methyl]-3-(6-difluoromethylpyrimidin-4-yl)-1,2,4-oxadiazol-5-one (hereinafter referred to as the present compound (8)).

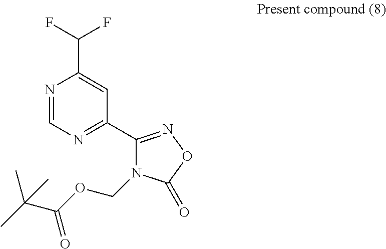

¹H-NMR: 1.10(s,9H), 6.16(s,2H), 6.66(t,1H), 8.30(s,1H), 9.41(s,1H)

### Production Example 9

To 6 ml of tetrahydrofuran, 0.85 g of 6-(1,1,2,2,2-pentafluoroethyl)pyrimidine-4-carboxamide oxime and 0.75 g of 1,1′-carbonyldiimidazole were added. This mixture was stirred at room temperature for 3 hours and a half. To this mixture, 0.71 g of 1,8-diazabicyclo[5.4.0]undec-7-ene was added, followed by stirring at room temperature for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.77 g of 3-[6-(1,1,2,2,2-pentafluoroethyl)pyrimidin-4-yl]-1,2,4-oxadiazol-5-one (hereinafter referred to as the present compound (9)).

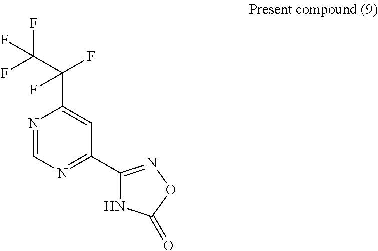

¹H-NMR (DMSO-d₆): 8.42(s,1H), 9.72(s,1H)

### Production Example 10

To 25 ml of tetrahydrofuran, 3.15 g of 2-cyclopropyl-6-trifluoromethylpyrimidine-4-carboxamide oxime and 2.9 g of 1,1′-carbonyldiimidazole were added. This mixture was stirred at room temperature for 1 hour and a half. To this mixture, 2.73 g of 1,8-diazabicyclot5.4.0]undec-7-ene was added, followed by stirring at room temperature for 4 hours. To the reaction mixed solution, water and 10% hydrochloric acid were added, followed by extraction three times with ethyl acetate. The organic layer was dried over anhydrous magnesium sulfate and then concentrated. The residue was subjected to silica gel column chromatography to obtain 3.2 g of 3-(2-cyclopropyl-6-trifluoromethylpyrimidin-4-yl)-1,2,4-oxadiazol-5-one (hereinafter referred to as the present compound (10)).

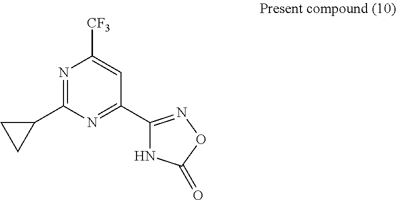

¹H-NMR (DMSO-d₆): 1.22-1.25(m,4H), 2.36-2.43(m,1H), 8.06(s,1H), 13.47(bs,1H)

### Production Example 11

To a mixture of 2 ml of pyridine, 0.15 g of 1,8-diazabicyclo[5.4.0]undec-7-ene and 0.2 g of 3-[6-(1,1,2,2,2-pentafluoroethyl)pyrimidin-4-yl]-1,2,4-oxadiazol-5-one, 0.18 g of 1-pyrrolidinecarbonyl chloride was added at room temperature. This mixture was stirred at 60° C. for 6 hours. The reaction mixture was left standing to cool to room temperature and then concentrated. The residue was subjected to silica gel column chromatography to obtain 0.14 g of 4-(1-pyrrolidinecarbonyl)-3-[6-(1,1,2,2,2-pentafluoroethyl)pyrimidin-4-yl]-1,2,4-oxadiazol-5-one (hereinafter referred to as the present compound (11)).

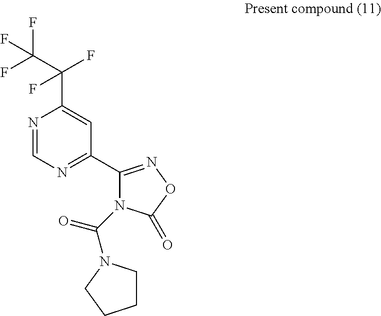

¹H-NMR: 2.05-2.10(m,4H), 3.64-3.73(m,4H), 8.28(s,1H), 9.46 (m, 1H)

### Production Example 12

To a mixture of 2 ml of pyridine, 0.16 g of 1,8-diazabicyclo[5.4.0]undec-7-ene and 0.2 g of 3-(2-cyclopropyl-6-trifluoromethylpyrimidin-4-yl)-1,2,4-oxadiazol-5-one, 0.16 g of 1-pyrrolidinecarbonyl chloride was added at room temperature. This mixture was stirred at 60° C. for 7 hours. The reaction mixture was left standing to cool to room temperature and then concentrated. The residue was subjected to silica gel column chromatography to obtain 0.17 g of 3-(2-cyclopropyl-6-trifluoromethylpyrimidin-4-yl)-4-(1-pyrrolidinecarbonyl)-1,2,4-oxadiazol-5-one (hereinafter referred to as the present compound (12)).

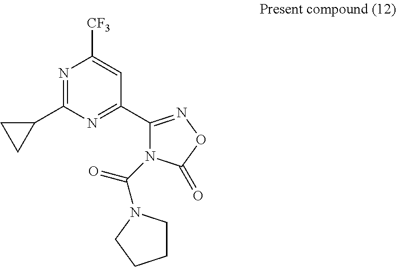

¹H-NMR: 1.10-1.16(m,2H), 1.22-1.27(m,2H), 2.07-2.12(m,4H), 2.37-2.41(m,1H), 3.65-3.70(m,4H), 7.94(s,1H)

### Production Example 13

0.04 g of sodium hydride (60% in oil) was suspended in 2 ml of N,N-dimethylformamide, and 0.2 g of 3-(2-cyclopropyl-6-trifluoromethylpyrimidin-4-yl)-1,2,4-oxadiazol-5-one was added thereto at room temperature. This mixture was stirred at room temperature for 10 minutes. To the mixture, 0.16 g of chloromethyl pivalate was added. This mixture was stirred at 80° C. for 6 hours. The reaction mixture was left standing to cool to room temperature. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 0.12 g of 4-[(2,2-dimethyl-1-oxopropoxy)methyl]-3-(2-cyclopropyl-6-trifluoromethylpyrimidin-4-yl)-1,2,4-oxadiazol-5-one (hereinafter referred to as the present compound (13)).

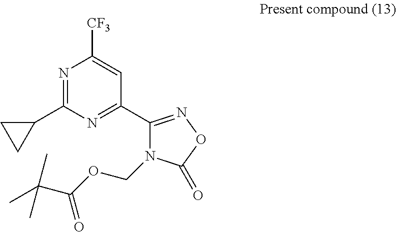

¹H-NMR: 1.11(s,9H), 1.20-1.28(m,4H), 2.38-2.46(m,1H), 6.15(s,2H), 8.02(s,1H)

### Production Example 14

To 1.5 ml of ethanol, 0.11 g of triethylamine, 0.2 g of 3-(2-cyclopropyl-6-trifluoromethylpyrimidin-4-yl)-1,2,4-oxadiazol-5-one and 0.07 g of acrolein were added. This mixture was stirred at room temperature for 18 hours. The reaction mixture was concentrated. The residue was subjected to silica gel column chromatography to obtain 0.07 g of 3-[3-(2-cyclopropyl-6-trifluoromethylpyrimidin-4-yl)-1,2,4-oxadiazol-5-one-4-yl]propionaldehyde (hereinafter referred to as the present compound (14)).

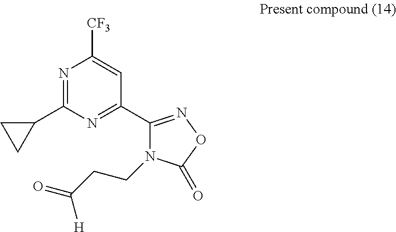

¹H-NMR: 1.18-1.30(m,4H), 2.37-2.42(m,1H), 3.08(t,2H), 4.52(t,2H), 8.02(s,1H), 9.81(s,1H)

### Production Example 15

To 12 ml of tetrahydrofuran, 1.5 g of 2-isopropyl-6-trifluoromethylpyrimidine-4-carboxamide oxime and 1.4 g of 1,1′-carbonyldiimidazole were added. This mixture was stirred at room temperature for 2 hours. To this mixture, 1.3 g of 1,8-diazabicyclo[5.4.0]undec-7-ene was added, followed by stirring at room temperature for 4 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1.5 g of 3-(2-isopropyl-6-trifluoromethylpyrimidin-4-yl)-1,2,4-oxadiazol-5-one (hereinafter referred to as the present compound (15)).

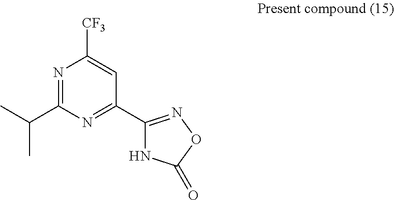

¹H-NMR (DMSO-d₆): 1.36(d,6H), 3.31-3.34(m,1H), 8.18(s,1H), 13.51(bs,1H)

### Production Example 16

To a mixture of 1.5 ml of pyridine, 0.16 g of 1,8-diazabicyclo[5.4.0]undec-7-ene and 0.2 g of 3-(2-isopropyl-6-trifluoromethylpyrimidin-4-yl)-1,2,4-oxadiazol-5-one, 0.14 g of 1-pyrrolidinecarbonyl chloride was added at room temperature. This mixture was stirred at 60° C. for 30 minutes and then stirred at 100° C. for 2 hours. The reaction mixture was left standing to cool to room temperature and then concentrated. The residue was subjected to silica gel column chromatography to obtain 0.2 g of 4-(1-pyrrolidinecarbonyl)-3-(2-isopropyl-6-trifluoromethylpyrimidin-4-yl)-1,2,4-oxadiazoi-5-one (hereinafter referred to as the present compound (16)).

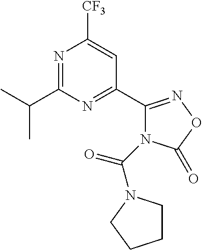

¹H-NMR: 1.21(d,6H), 2.07-2.10(m,4H), 3.29-3.39(m,1H), 3.63-3.74(m,4H), 8.03(s,1H)

### Production Example 17

To 1.5 ml of ethanol, 0.12 g of triethylamine, 0.2 g of 3-(2-isopropyl-6-trifluoromethylpyrimidin-4-yl)-1,2,4-oxadiazol-5-one and 0.06 g of acrolein were added. This mixture was stirred at room temperature for 18 hours. The reaction mixture was concentrated. The residue was subjected to silica gel column chromatography to obtain 0.12 g of 3-[3-(2-isopropyl-6-trifluoromethylpyrimidin-4-yl)-1,2,4-oxadiazol-5-one-4-yl]propionaldehyde (hereinafter referred to as the present compound (17)).

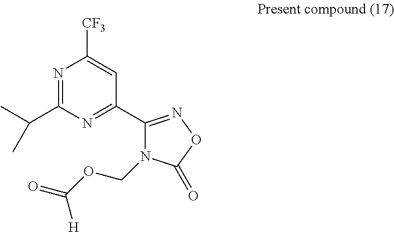

¹H-NMR: 1.39(d,6H), 3.09(t,2H), 3.36-3.43(m,1H), 4.58(t,2H), 8.13(s,1H), 9.79(s,1H)

### Production Example 18

To 3 ml of tetrahydrofuran, 0.51 g of 6-(1,1,2,2,3,3,3-heptafluoropropyl)pyrimidine-4-carboxamide oxime and 0.39 g of 1,1′-carbonyldiimidazole were added. This mixture was stirred at room temperature for 2 hours. To this mixture, 0.36 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.45 g of 3-[6-(1,1,2,2,3,3,3-heptafluoropropyl)pyrimidin-4-yl]-1,2,4-oxadiazol-5-one (hereinafter referred to as the present compound (18)).

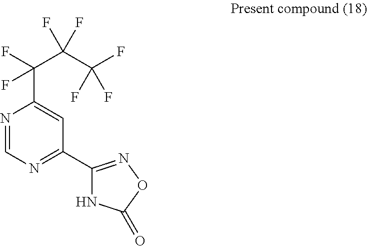

¹H-NMR (DMSO-d₆): 8.40(s,1H), 9.73(s,1H)

### Production Example 19

To 10 ml of tetrahydrofuran, 1.4 g of 6-trifluoromethyl-5-bromopyrimidine-4-carboxamide oxime and 1.1 g of 1,1′-carbonyldiimidazole were added. This mixture was stirred at room temperature for 2 hours and a half. To this mixture, 1.1 g of 1,8-diazabicyclo[5.4.0]undec-7-ene was added, followed by stirring at room temperature for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1.5 g of 3-(6-trifluoromethyl-5-bromopyrimidin-4-yl)-1,2,4-oxadiazol-5-one (hereinafter referred to as the present compound (19)).

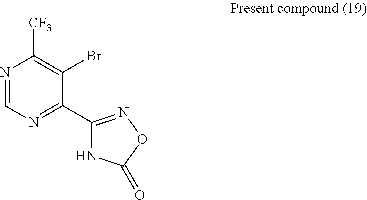

¹H-NMR (DMSO-d₆): 9.57(s,1H)

### Production Example 20

To 8 ml of tetrahydrofuran, 1 g of 6-(4-trifluoromethylphenyl)pyrimidine-4-carboxamide oxime and 0.85 g of 1,1′-carbonyldiimidazole were added. This mixture was stirred at room temperature for 2 hours and a half. To this mixture, 0.79 g of 1,8-diazabicyclo[5.4.0]undec-7-ene was added, followed by stirring at room temperature for 10 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.8 g of 3-[6-(4-trifluoromethylphenyl)pyrimidin-4-yl]-1,2,4-oxadiazol-5-one (hereinafter referred to as the present compound (20)).

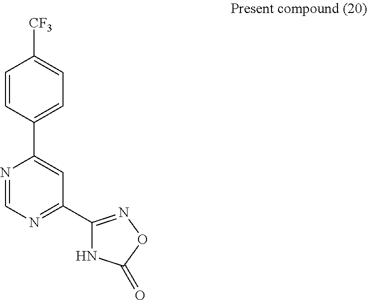

¹H-NMR (DMSO-d₆): 7.98(d,2H), 8.51(d,2H), 8.62(s,1H), 9.53(s,1H), 13.52(bs,1H)

### Production Example 21

To a mixture of 1 ml of pyridine, 0.14 g of 1,8-diazabicyclo[5.4.0]undec-7-ene and 0.2 g of 3-(6-trifluoromethyl-5-bromopyrimidin-4-yl)-1,2,4-oxadiazol-5-one, 0.12 g of 1-pyrrolidinecarbonyl chloride was added at room temperature. This mixture was stirred at 70° C. for 2 hours. The reaction mixture was concentrated. The residue was subjected to silica gel column chromatography to obtain 0.05 g of 4-(1-pyrrolidinecarbonyl)-3-(6-trifluoromethyl-5-bromopyrimidin-4-yl)-1,2,4-oxadiazol-5-one (hereinafter referred to as the present compound (21)).

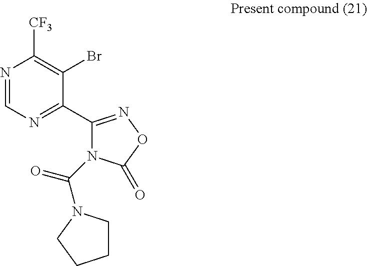

¹H-NMR: 2.02-2.07(m,4H), 3.53-3.56(m,2H), 3.77-3.80(m,2H), 9.28(s,1H)

### Production Example 22

To 2 ml of tetrahydrofuran, 0.3 g of 2,6-bis(trifluoromethyl)pyrimidine-4-carboxamide oxime and 0.25 g of 1,1′-carbonyldiimidazole were added. This mixture was stirred at room temperature for 4 hours and a half. To this mixture, 0.23 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.27 g of 3-[2,6-bis(trifluoromethyl)pyrimidin-4-yl]-1,2,4-oxadiazol-5-one (hereinafter referred to as the present compound (22)).

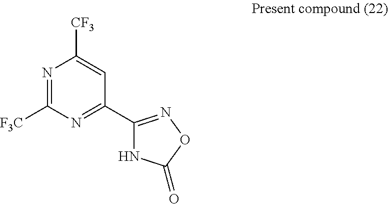

¹H-NMR (DMSO-d₆) 8.69(s,1H)

### Production Example 23

To 1.5 ml of ethanol, 0.08 g of triethylamine, 0.2 g of 3-(6-trifluoromethyl-5-bromopyrimidin-4-yl)-1,2,4-oxadiazol-5-one and 0.05 g of acrolein were added. This mixture was stirred at room temperature for 18 hours. The reaction mixture was concentrated. The residue was subjected to silica gel column chromatography to obtain 0.09 g of 3-[3-(6-trifluoromethyl-5-bromopyrimidin-4-yl)-1,2,4-oxadiazol-5-one-4-yl]propionaldehyde (hereinafter referred to as the present compound (23)).

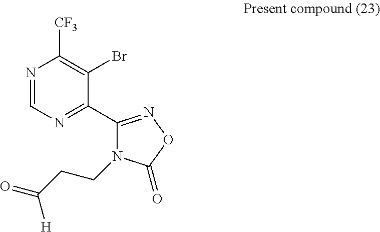

¹H-NMR: 3.04(t,2H), 4.08(t,2H), 9.36(s,1H), 9.65(s,1H)

### Production Example 24

To a mixture of 3 ml of water, 0.34 g of sodium cyanide and 0.07 g of 1,4-diazabicyclo[2.2.2]octane, 9 ml of dimethyl sulfoxide and 1.5 g of 4-chloro-6-(3-trifluoromethylphenyl)pyrimidine were added at room temperature. This mixture was stirred at 3 hours. Water was poured into the reaction mixture, followed by extraction three times with tent-butyl methyl ether. The organic layer was washed with saturated brine, dried over anhydrous magnesium sulfate and then concentrated to obtain a crude product. This crude product was used in the next step without purification.

To 10 ml of ethanol, 1 g of sodium hydrogen carbonate and 0.81 g of hydroxylamine hydrochloride were added. This mixture was heated under reflux for 1 hour. This mixture was left standing to cool to 0° C. To this mixture, the crude product was added, followed by stirring at 0° C. for 30 minutes and further stirring at room temperature for 5 hours. The reaction mixture was concentrated. To the residue, water was added, followed by extraction three times with ethyl acetate. The organic layer was washed with saturated brine, dried over anhydrous magnesium sulfate and then concentrated to obtain a crude product. This crude product was used in the next step without purification.

To 10 ml of N,N-dimethylformamide, the crude product and 1 g of 1,1′-carbonyldiimidazole were added. This mixture was stirred at room temperature for 1 hour and a half. To this mixture, 0.98 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.9 g of 3-[6-(3-trifluoromethylphenyl)pyrimidin-4-yl]-1,2,4-oxadiazol-5-one (hereinafter referred to as the present compound (24)).

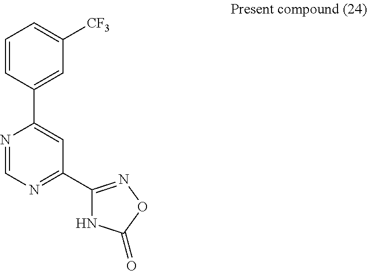

¹H-NMR (DMSO-d₆): 7.85(t,1H), 8.00(d,1H), 8.60-8.63(m,2H), 8.68(s,1H), 9.51(s,1H) 13.54(bs,1H)

### Production Example 25

To a mixture of 2 ml of pyridine, 0.14 g of 1,8-diazabicyclo[5.4.0]undec-7-ene and 0.2 g of 3-[6-(3-trifluoromethylphenyl)pyrimidin-4-yl]-1,2,4-oxadiazol-5-one, 0.12 g of 1-pyrrolidinecarbonyl chloride was added at room temperature. This mixture was stirred at 60° C. for 6 hours. The reaction mixture was concentrated. The residue was subjected to silica gel column chromatography to obtain 0.14 g of 4-(1-pyrrolidinecarbonyl)-3-[6-(3-trifluoromethylphenyl)pyrimidin-4-yl)-1,2,4-oxadiazol-5-one (hereinafter referred to as the present compound (25)).

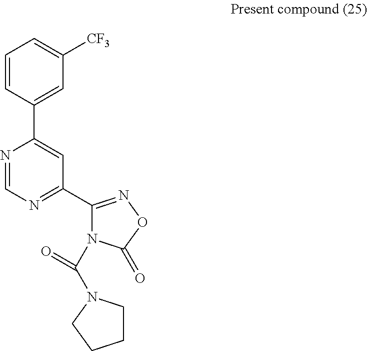

¹H-NMR: 2.07-2.10(m,4H), 3.65-3.81(m,4H), 7.70(t,1H), 7.84(d,1H), 8.31-8.33(m,2H), 8.46(s,1H), 9.33(s,1H)

### Production Example 26

To 5 ml of ethanol, 0.41 g of sodium hydrogen carbonate and 0.34 g of hydroxylamine hydrochloride were added. This mixture was heated under reflux for 1 hour. This mixture was cooled to 0° C. To this mixture, 6-(2,2,2-trifluoroethoxy)pyrimidine-4-carbonitrile was added, followed by stirring at 0° C. for 30 minutes and at room temperature for 2 hours. The reaction mixture was concentrated. To the residue, water was added, followed by extraction three times with ethyl acetate. The organic layer was washed with saturated brine, dried over anhydrous magnesium sulfate and then concentrated to obtain a crude product. This crude product was used in the next step without purification.

To 2 ml of tetrahydrofuran, the crude product and 0.56 g of 1,1′-carbonyldiimidazole were added. This mixture was stirred at room temperature for 4 hours and a half. To this mixture, 0.52 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.46 g of 3-[4-(2,2,2-trifluoroethoxy)pyrimidine-6-yl]-1,2,4-oxadiazol-5-one (hereinafter referred to as the present compound (26)).

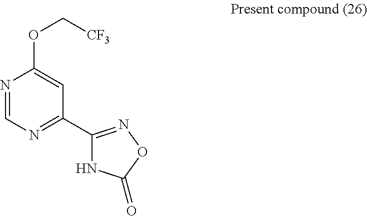

¹H-NMR (DMSO-d₆): 5.19(q,2H), 7.58(s,1H), 9.08(s,1H), 13.40(bs,1H)

### Production Example 27

To 7 ml of tetrahydrofuran, 1.1 g of 2-cyclopropyl-6-(1,1,2,2,2-pentafluoroethyl)pyrimidine-4-carboxamide oxime and 0.61 g of 1,1′-carbonyldiimidazole were added. This mixture was stirred at room temperature for 2 hours. To this mixture, 0.8 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1 g of 3-[2-cyclopropyl-6-(1,1,2,2,2-pentafluoroethyl)pyrimidin-4-yl]-1,2,4-oxadiazol-5-one (hereinafter referred to as the present compound (27)).

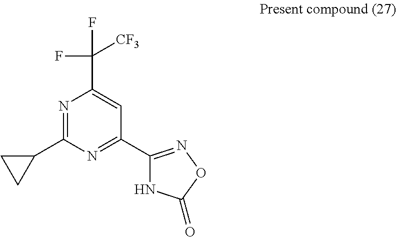

¹H-NMR (DMSO-d₆): 1.21-1.27(m,4H), 2.37-2.43(m,1H), 8.08(s,1H), 13.47(bs,1H)

### Production Example 28

To a mixture of 1 ml of pyridine, 0.13 g of 1,8-diazabicyclo[5.4.0]undec-7-ene and 0.2 g of 3-[2-cyclopropyl-6-(1,1,2,2,2-pentafluoroethyl)pyrimidin-4-yl]-1,2,4-oxadiazol-5-one, 0.12 g of 1-pyrrolidinecarbonyl chloride was added at room temperature. This mixture was stirred at 70° C. for 4 hours. The reaction mixture was concentrated. The residue was subjected to silica gel column chromatography to obtain 0.13 g of 3-[2-cyclopropyl-6-(1,1,2,2,2-pentafluoroethyl)pyrimidin-4-yl]-4-(1-pyrrolidinecarbonyl)-1,2,4-oxadiazol-5-one (hereinafter referred to as the present compound (28)).

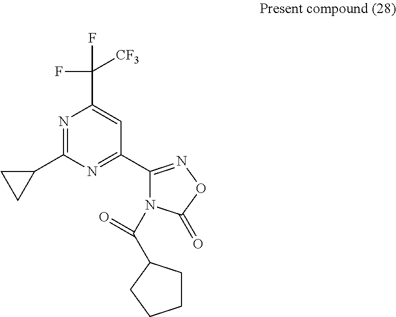

¹H-NMR: 1.12-1.16(m,2H), 1.23-1.28(m,2H), 2.07-2.12(m,4H), 2.35-2.40(m,1H), 3.65-3.71(m,4H), 7.98(s,1H)

### Production Example 29

To a mixture of 2 ml of tetrahydrofuran, 0.2 g of 3-(2-cyclopropyl-6-trifluoromethylpyrimidin-4-yl)-1,2,4-oxadiazol-5-one and 0.25 g of triphenylphosphine, 0.17 g of azodicarboxylic acid diethyl ester was added at 0° C. This mixture was stirred at 0° C. for 10 minutes. To the mixture, 0.07 g of cyclopropanemethanol was added, followed by stirring at 60° C. for 1 hour. The reaction mixture was left standing to cool to room temperature, poured into water and then extracted three times with tert-butyl methyl ether. The organic layer was washed with saturated brine, dried over anhydrous magnesium sulfate and then concentrated. The residue was subjected to silica gel column chromatography to obtain 0.2 g of 4-cyclopropylmethyl-3-(2-cyclopropyl-6-trifluoromethylpyrimidin-4-yl)-1,2,4-oxadiazol-5-one (hereinafter referred to as the present compound (29)).

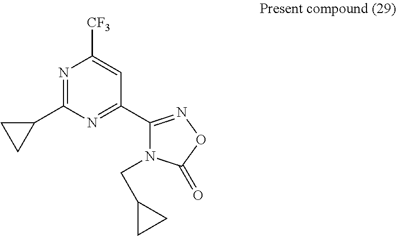

¹H-NMR: 0.43-0.48(m,2H), 0.54-0.59(m,2H), 1.24-1.33(m,5H), 2.44-2.50(m,1H), 4.07(d,2H), 8.06(s,1H)

### Production Example 30

To 15 ml of tetrahydrofuran, 2.2 g of 6-(2-trifluoromethylphenyl)pyrimidine-4-carboxamide oxime and 1.77 g of 1,1′-carbonyldiimidazole were added. This mixture was stirred at room temperature for 2 hours. To this mixture, 1.66 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 2.2 g of 3-[6-(2-trifluoromethylphenyl)pyrimidin-4-yl]-1,2,4-oxadiazol-5-one (hereinafter referred to as the present compound (30)).

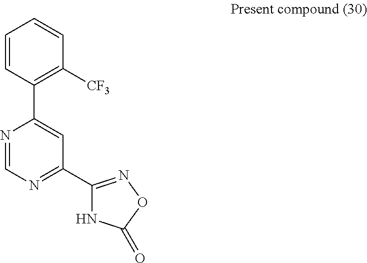

¹H-NMR (DMSO-d₅): 7.71(d,1H), 7.78-7.80(m,1H), 7.85-7.89(m,1H) 7.96(d,1H), 8.15(s,1H), 9.52(s,1H), 13.57(bs,1H)

### Production Example 31

To a mixture of 2 ml of tetrahydrofuran, 0.2 g of 3-(2-cyclopropyl-6-trifluoromethylpyrimidin-4-yl)-1,2,4-oxadiazol-5-one and 0.25 g of triphenylphosphine, 0.17 g of azodicarboxylic acid diethyl ester was added at 0° C. This mixture was stirred at 0° C. for 10 minutes. 0.1 g of cyclopentanemethanol was added thereto, followed by stirring at 60° C. for 3 hours. The reaction mixture was left standing to cool to room temperature, poured into water and then extracted three times with tent-butyl methyl ether. The organic layer was washed with saturated brine, dried over anhydrous magnesium sulfate and then concentrated. The residue was subjected to silica gel column chromatography to obtain 0.16 g of 4-cyclopentylmethyl-3-(2-cyclopropyl-6-trifluoromethylpyrimidin-4-yl)-1,2,4-oxadiazol-5-one (hereinafter referred to as the present compound (31)).

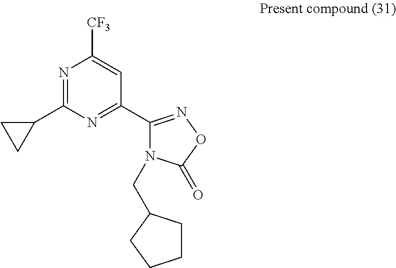

H-NMR: 1.22-1.32(m,6H), 1.52-1.70(m,6H), 2.25-2.32(m,1H), 2.42-2.47(m,1H), 4.15(d,2H), 8.05(s,1H)

### Production Example 33

To 9 ml of tetrahydrofuran, 1 g of 2-methyl-6-trifluoromethylpyrimidine-4-carboxamide oxime and 0.96 g of 1,1′-carbonyldiimidazole were added. This mixture was stirred at room temperature for 2 hours. To this mixture, 0.9 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.9 g of 3-(2-methyl-6-trifluoromethylpyrimidin-4-yl)-1,2,4-oxadiazol-5-one (hereinafter referred to as the present compound (33)).

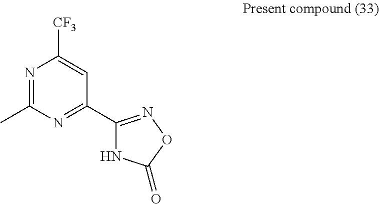

¹H-NMR (DMSO-d₆): 2.84(s,3H), 8.19(s,1H)

### Production Example 34

To 5 ml of tetrahydrofuran, 0.62 g of 2-methoxy-6-trifluoromethylpyrimidine-4-carboxamide oxime and 0.55 g of 1,1′-carbonyldiimidazole were added. This mixture was stirred at room temperature for 3 hours. To this mixture, 0.52 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.54 g of 3-(2-methoxy-6-trifluoromethylpyrimidin-4-yl)-1,2,4-oxadiazol-5-one (hereinafter referred to as the present compound (34)).

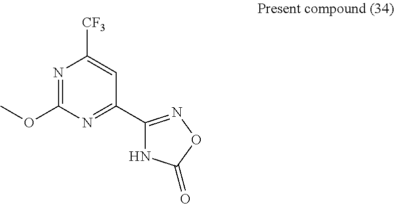

¹H-NMR (DMSO-d₆): 4.10(s,3H), 7.97(s,1H)

### Production Example 35

0.06 g of sodium hydride (60% in oil) was suspended in 2 ml of N,N-dimethylformamide and 0.3 g of 3-(2-cyclopropyl-6-trifluoromethylpyrimidin-4-yl)-1,2,4-oxadiazol-5-one was added at room temperature. This mixture was stirred at for 10 minutes. 0.24 g of chloromethylbenzoate was added thereto. This mixture was stirred at 60° C. for 4 hours. The reaction mixture was left standing to cool to room temperature. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 0.23 g of [3-(2-cyclopropyl-6-trifluoromethylpyrimidin-4-yl)-1,2,4-oxadiazol-5-one-4-yl]methyl=benzoate (hereinafter referred to as the present compound (35)).

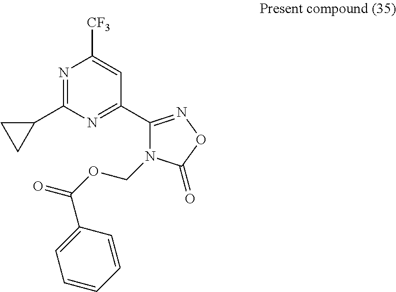

¹H-NMR: 1.19-1.26(m,4H), 2.38-2.44(m,1H), 6.40(s,2H), 7.42(t,2H), 7.59(dd,1H), 7.93(dd,2H), 8.03(s,1H)

### Production Example 36

To 10 ml of tetrahydrofuran, 1.6 g of 2-phenyl-6-trifluoromethylpyrimidine-4-carboxamide oxime and 1.3 g of 1,1′-carbonyldiimidazole were added. This mixture was stirred at room temperature for 2 hours. To this mixture, 1.2 g of 1,8-diazabicyclo[5.4.0]undec-7-ene was added, followed by stirring for 4 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1.5 g of 3-(2-phenyl-6-trifluoromethylpyrimidin-4-yl)-1,2,4-oxadiazol-5-one (hereinafter referred to as the present compound (36)).

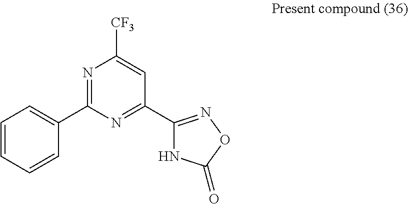

¹H-NMR (DMSO-d₆): 7.61-7.67(m,3H), 8.25(s,1H), 8.62-8.66(m,2H), 13.68(bs,1H)

### Production Example 37

To a mixture of 2 ml of pyridine, 0.19 g of 1,8-diazabicyclo[5.4.0]undec-7-ene and 0.3 g of 3-(2-phenyl-6-trifluoromethylpyrimidin-4-yl)-1,2,4-oxadiazol-5-one, 0.17 g of 1-pyrrolidinecarbonyl chloride was added at room temperature. This mixture was stirred at 70° C. for 6 hours. The reaction mixture was left standing to cool to room temperature and then concentrated. The residue was subjected to silica gel column chromatography to obtain 0.2 g of 3-(2-phenyl-6-trifluoromethylpyrimidin-4-yl)-4-(1-pyrrolidinecarbonyl)-1,2,4-oxadiazol-5-one (hereinafter referred to as the present compound (37)).

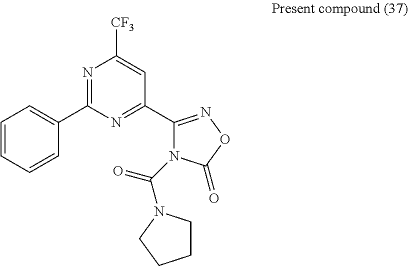

¹H-NMR: 2.10-2.14(m,4H), 3.72-3.79(m,4H), 7.49-7.61(m,3H), 8.08(s,1H), 8.35-8.38(m,2H)

### Production Example 38

To 10 ml of tetrahydrofuran, 1 g of 2-methylthio-6-trifluoromethylpyrimidine-4-carboxamide oxime and 0.96 g of 1,1′-carbonyldiimidazole were added. This mixture was stirred at room temperature for 30 minutes. To this mixture, 0.9 g of 1,8-diazabicyclo[5.4.0]undec-7-ene was added, followed by stirring for 4 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 1 g of 3-(2-methylthio-6-trifluoromethylpyrimidin-4-yl)-1,2,4-oxadiazol-5-one (hereinafter referred to as the present compound (38)).

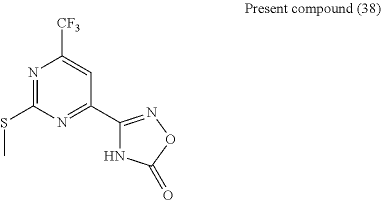

¹H-NMR (DMSO-d₆): 2.67(s,3H), 7.99(s,1H)

### Production Example 39

To 3 ml of tetrahydrofuran, 0.33 g of 6-[N-methyl-N-(2,2,2-trifluoroethyl)amino]pyrimidine-4-carboxamide oxime and 0.3 g of 1,1′-carbonyldiimidazole were added. This mixture was stirred at room temperature for 4 hours and a half. To this mixture, 0.28 g of 1,8-diazabicyclo[5.4.0]undec-7-ene was added, followed by stirring for 4 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.3 g of 3-{6-[N-methyl-N-(2,2,2-trifluoroethyl)amino]pyrimidin-4-yl}-1,2,4-oxadiazol-5-one (hereinafter referred to as the present compound (39)).

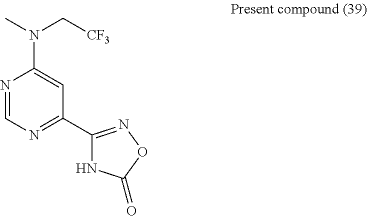

¹H-NMR (DMSO-d₆): 3.21(s,3H), 4.62(q,2H), 7.31(s,1H), 8.76(s,1H)

### Production Example 40

To 5 ml of tetrahydrofuran, 0.58 g of 2-methyl-6-phenylpyrimidine-4-carboxamide oxime and 0.54 g of 1,1′-carbonyldiimidazole were added. This mixture was stirred at room temperature for 1 hour. To this mixture, 0.5 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.59 g of 3-(2-methyl-6-phenylpyrimidin-4-yl)-1,2,4-oxadiazol-5-one (hereinafter referred to as the present compound (40)).

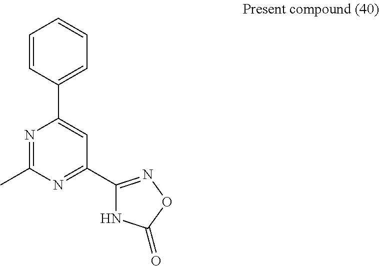

¹H-NMR (DMSO-d₆): 2.80(s,3H), 7.57-7.64(m,3H), 8.25-8.29(m,2H), 8.30(s,1H), 13.36(bs,1H)

### Production Example 41

To 3 ml of tetrahydrofuran, 0.33 g of 2-methyl-6-(3-chlorophenyl)pyrimidine-4-carboxamide oxime and 0.26 g of 1,1′-carbonyldiimidazole were added. This mixture was stirred at room temperature for 1 hour. To this mixture, 0.24 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.32 g of 3-[2-methyl-6-(3-chlorophenyl)pyrimidin-4-yl]-1,2,4-oxadiazol-5-one (hereinafter referred to as the present compound (41)).

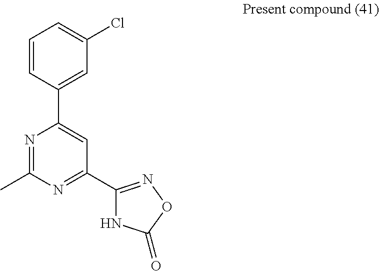

¹H-NMR (DMSO-d₆): 2.81(s,3H), 7.62(t,1H), 7.68-7.70(m,1H), 8.24(d,1H), 8.32-8.33(m,1H), 8.38(s,1H), 13.39(bs,1H)

### Production Example 42

To 2 ml of tetrahydrofuran, 0.21 g of 2-methyl-6-(2-chlorophenyl)pyrimidine-4-carboxamide oxime and 0.17 g of 1,1′-carbonyldiimidazole were added. This mixture was stirred at room temperature for 1 hour. To this mixture, 0.16 g of 1,8-diazabicyclo[5.4.0]undec-7-ene was added, followed by stirring for 2 hours and a half. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.21 g of 3-[2-methyl-6-(2-chlorophenyl)pyrimidin-4-yl]-1,2,4-oxadiazol-5-one (hereinafter referred to as the present compound (42)).

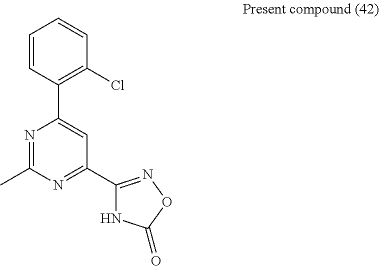

¹H-NMR (DMSO-d₆): 2.80(s,3H), 7.52-7.61(m,2H), 7.66(dd,1H), 7.72(dd,1H), 8.07(s,1H), 13.43(bs,1H)

### Production Example 43

To 4 ml of tetrahydrofuran, 0.43 g of 2-methyl-6-(4-chlorophenyl)pyrimidine-4-carboxamide oxime and 0.39 g of 1,1′-carbonyldiimidazole were added. This mixture was stirred at room temperature for 1 hour. To this mixture, 0.36 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.43 g of 3-[2-methyl-6-(4-chlorophenyl)pyrimidin-4-yl]-1,2,4-oxadiazol-5-one (hereinafter referred to as the present compound (43)).

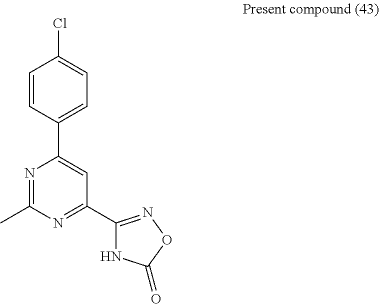

¹H-NMR (DMSO-d₆): 2.79(s,3H), 7.64-7.68(m,2H), 8.28-8.33(m,3H), 13.35(bs,1H)

### Production Example 44

To 3 ml of tetrahydrofuran, 0.29 g of 2-methyl-6-(3-fluorophenyl)pyrimidine-4-carboxamide oxime and 0.25 g of 1,1′-carbonyldiimidazole were added. This mixture was stirred at room temperature for 3 hours. To this mixture, 0.23 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.28 g of 3-[2-methyl-6-(3-fluorophenyl)pyrimidin-4-yl]-1,2,4-oxadiazol-5-one (hereinafter referred to as the present compound (44)).

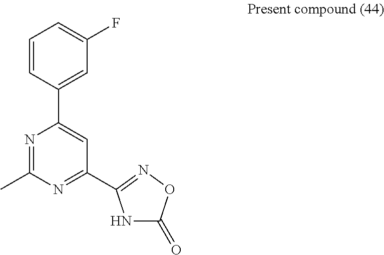

¹H-NMR (DMSO-d₆): 2.80(s,3H), 7.46(td,1H), 7.61-7.67(m,1H), 8.08(d,1H), 8.13(d,1H), 8.35(s,1H)

### Production Example 45

To 3 ml of tetrahydrofuran, 0.28 g of 2-methyl-6-(2-fluorophenyl)pyrimidine-4-carboxamide oxime and 0.24 g of 1,1′-carbonyldiimidazole were added. This mixture was stirred at room temperature for 1 hour. To this mixture, 0.23 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.25 g of 3-[2-methyl-6-(2-fluorophenyl)pyrimidin-4-yl]-1,2,4-oxadiazol-5-one (hereinafter referred to as the present compound (45)).

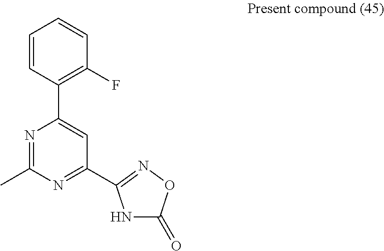

¹H-NMR (DMSO-d₆): 2.81(s,3H), 7.42-7.48(m,2H), 7.63-7.69(m,1H), 8.13-8.18(m,2H), 13.45 (bs, 1H)

### Production Example 46

To 3 ml of tetrahydrofuran, 0.29 g of 2-methyl-6-(4-methylphenyl)pyrimidine-4-carboxamide oxime and 0.25 g of 1,1′-carbonyldiimidazole were added. This mixture was stirred at room temperature for 3 hours. To this mixture, 0.23 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.28 g of 3-[2-methyl-6-(4-methylphenyl)pyrimidin-4-yl]-1,2,4-oxadiazol-5-one (hereinafter referred to as the present compound (46)).

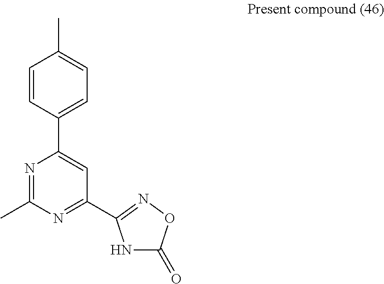

¹H-NMR (DMSO-d₆): 2.40(s,3H), 2.77(s,3H), 7.39(d,2H), 8.17(d,2H), 8.24(s,1H)

### Production Example 47

To 3 ml of tetrahydrofuran, 0.3 g of 2-methyl-6-[3-methylphenyl)pyrimidine-4-carboxamide oxime and 0.26 g of 1,1′-carbonyldiimidazole were added. This mixture was stirred at room temperature for 3 hours. To this mixture, 0.24 g of 1,8-diazabicyclo[5.4.0]undec-7-ene was added, followed by stirring for 30 minutes. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.29 g of 3-[2-methyl-6-(3-methylphenyl)pyrimidin-4-yl]-1,2,4-oxadiazol-5-one (hereinafter referred to as the present compound (47)).

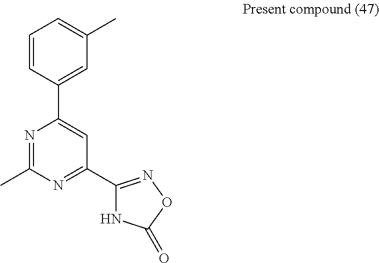

¹H-NMR (DMSO-d₆): 2.40(s,3H), 2.78(s,3H),7.42(d,1H), 7.47(t,1H), 8.05(d,1H), 8.09(s,1H), 8.27(s,1H)

### Production Example 48

To 4 ml of tetrahydrofuran, 0.47 g of 2-methyl-6-(4-fluorophenyl)pyrimidine-4-carboxamide oxime and 0.4 g of 1,1′-carbonyldiimidazole were added. This mixture was stirred at room temperature for 1 hour and a half. To this mixture, 0.38 g of 1,8-diazabicyclo[5.4.0]undec-7-ene was added, followed by stirring for 30 minutes. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.35 g of 3-[2-methyl-6-(4-fluorophenyl)pyrimidin-4-yl]-1,2,4-oxadiazol-5-one (hereinafter referred to as the present compound (48)).

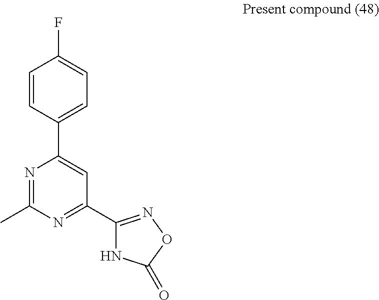

¹H-NMR (DMSO-d₆): 2.79(s,3H), 7.39-7.45(m,2H), 8.30(s,1H), 8 .33-8.37 (m, 2H)

### Production Example 49

To 4 ml of tetrahydrofuran, 0.26 g of 2-methyl-6-(2-methylphenyl)pyrimidine-4-carboxamide oxime and 0.23 g of 1,1′-carbonyldiimidazole were added. This mixture was stirred at room temperature for 3 hours and a half. To this mixture, 0.21 g of 1,8-diazabicyclo[5.4.0]undec-7-ene was added, followed by stirring for 1 hour. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.23 g of 3-[2-methyl-6-(2-methylphenyl)pyrimidin-4-yl]-1,2,4-oxadiazol-5-one (hereinafter referred to as the present compound (49)).

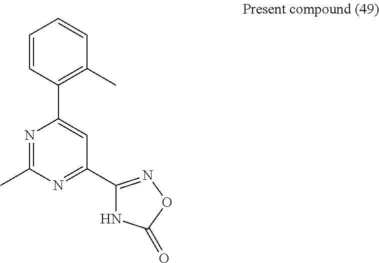

¹H-NMR (DMSO-d₅): 2.42(s,3H), 2.78(s,3H), 7.35-7.47(m,3H), 7.55(d,1H), 7.89(s,1H)

### Production Example 50

To 4 ml of tetrahydrofuran, 0.45 g of 2-methyl-6-benzyloxypyrimidine-4-carboxamide oxime and 0.37 g of 1,1′-carbonyldiimidazole were added. This mixture was stirred at room temperature for 3 hours. To this mixture, 0.35 g of 1,8-diazabicyclo[5.4.0]undec-7-ene was added, followed by stirring for 30 minutes. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.41 g of 3-[2-methyl-6-benzyloxypyrimidin-4-yl]-1,2,4-oxadiazol-5-one (hereinafter referred to as the present compound (50)).

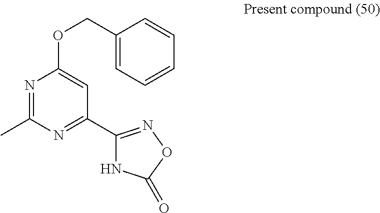

¹H-NMR (DMSO-d₆): 2.63(s,3H), 5.48(s,2H), 7.22(s,1H), 7.34-7.44(m,3H), 7.49(d,2H)

### Production Example 51

To 3 ml of tetrahydrofuran, 0.16 g of 2-cyclopropyl-6-phenylpyrimidine-4-carboxamide oxime and 0.14 g of 1,1′-carbonyldiimidazole were added. This mixture was stirred at room temperature for 3 hours. To this mixture, 0.35 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.14 g of 3-(2-cyclopropyl-6-phenylpyrimidin-4-yl)-1,2,4-oxadiazol-5-one (hereinafter referred to as the present compound (51)).

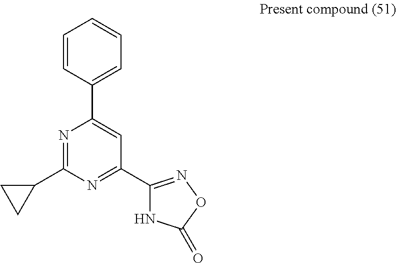

¹H-NMR (DMSO-d₆): 1.12-1.25(m,4H), 2.32-2.38(m,1H), 7.55-7.61(m,3H), 8.19(s,1H), 8.22-8.26(m,2H), 10.28(bs,1H)

### Production Example 52

To 2 ml of tetrahydrofuran, 0.24 g of 2,5-dimethyl-6-phenylpyrimidine-4-carboxamide oxime and 0.21 g of 1,1′-carbonyldiimidazole were added. This mixture was stirred at room temperature for 2 hours. To this mixture, 0.19 g of 1,8-diazabicyclo[5.4.0]undec-7-ene was added, followed by stirring for 1 hour. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.23 g of 3-(2,5-dimethyl-6-phenylpyrimidin-4-yl)-1,2,4-oxadiazol-5-one (hereinafter referred to as the present compound (52)).

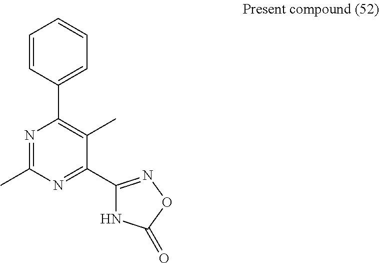

¹H-NMR (DMSO-d₆): 2.46(s,3H), 2.71(s,3H), 7.53-7.56(m,3H), 7.59-7.63(m,2H), 13.02(bs,1H)

### Production Example 53

To 5 ml of tetrahydrofuran, 0.56 g of 5-methyl-6-phenylpyrimidine-4-carboxamide oxime and 0.52 g of 1,1′-carbonyldiimidazole were added. This mixture was stirred at room temperature for 2 hours. To this mixture, 0.49 g of 1,8-diazabicyclo[5.4.0]undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.56 g of 3-(5-methyl-6-phenylpyrimidin-4-yl)-1,2,4-oxadiazol-5-one (hereinafter referred to as the present compound (53)).

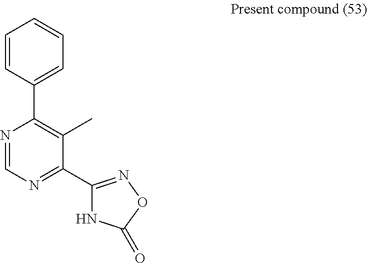

¹H-NMR (DMSO-d₆): 2.53(s,3H), 7.54-7.57(m,3H), 7.63-7.67(m,2H), 9.27(s,1H), 13.24(bs,1H)

### Production Example 54

To 3 ml of ethanol, 0.14 g of sodium hydrogen carbonate and 0.11 g of hydroxylamine hydrochloride were added. This mixture was heated under reflux for 1 hour. After cooling this mixture to 0° C., 0.23 g of 2-methyl-6-benzylpyrimidine-4-carbonitrile was added to this mixture. This mixture was stirred at 0° C. for 30 minutes and then stirred at room temperature for 3 hours. The reaction mixture was concentrated. To the residue, water was added, followed by extraction three times with ethyl acetate. The organic layer was washed with saturated brine, dried over anhydrous magnesium sulfate and then concentrated to obtain a crude product. This crude product was used in the next step without purification.

To 3 ml of tetrahydrofuran, the crude product and 0.26 g of 1,1′-carbonyldiimidazole were added. This mixture was stirred at room temperature for 1 hour and a half. To this mixture, 0.25 g of 1,8-diazabicyclo[5.4.01undec-7-ene was added, followed by stirring for 2 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.24 g of 3-(2-methyl-6-benzylpyrimidin-4-yl)-1,2,4-oxadiazol-5-one (hereinafter referred to as the present compound (54)).

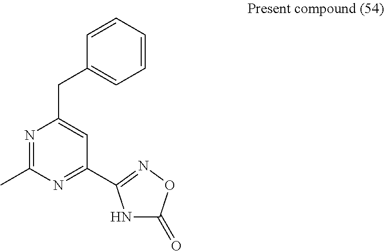

¹H-NMR (DMSO-d₆): 2.69(s,3H), 4.17(s,2H), 7.23-7.36(m,5H), 7.68(s,1H), 13.30(bs,1H)

### Production Example 55

To 4 ml of ethanol, 0.25 g of sodium hydrogen carbonate and 0.14 g of hydroxylamine hydrochloride were added. This mixture was heated under reflux for 1 hour. After cooling this mixture to 0° C., 0.36 g of 2-methyl-6-(2-pyridyl)pyrimidine-4-carbonitrile was added to this mixture.

This mixture was stirred at 0° C. for 30 minutes, followed by stirring at room temperature for 3 hours. The reaction mixture was concentrated. To the residue, water was added, followed by extraction three times with ethyl acetate. The organic layer was washed with saturated brine, dried over anhydrous magnesium sulfate and then concentrated to obtain a crude product. This crude product was used in the next step without purification.

To 4 ml of tetrahydrofuran, the crude product and 0.47 g of 1,1′-carbonyldiimidazole were added. This mixture was stirred at room temperature for 1 hour and a half. To this mixture, 0.45 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.3 g of 3-[2-mettyl-6-(2-pyridyl)pyrimidin-4-yl]-1,2,4-oxadiazol-5-one (hereinafter referred to as the present compound (55)).

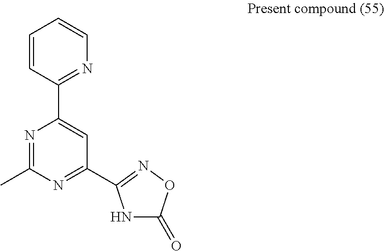

¹H-NMR (DMSO-d₆): 2.83(s,3H), 7.63-7.65(m,1H), 8.05-8.09(m,1H), 8.50(d,1H), 8.64(s,1H), 8.81(d,1H), 13.44(bs,1H)

### Production Example 56

To 2 ml of tetrahydrofuran, 0.2 g of 2-isopropyl-6-phenylpyrimidine-4-carboxamide oxime and 0.16 g of 1,1′-carbonyldiimidazole were added. This mixture was stirred at room temperature for 2 hours. To this mixture, 0.15 g of 1,8-diazabicyclo[5.4.0]undec-7-ene was added, followed by stirring for 3 hours. To the reaction mixture, water and 10% hydrochloric acid were added, followed by extraction three times with ethyl acetate. The organic layer was dried over anhydrous magnesium sulfate and then concentrated. The residue was subjected to silica gel column chromatography to obtain 0.18 g of 3-(2-isopropyl-6-phenylpyrimidin-4-yl)-1,2,4-oxadiazol-5-one (hereinafter referred to as the present compound (56)).

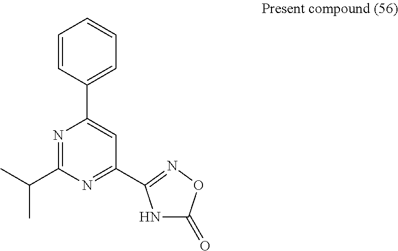

¹H-NMR (DMSO-d₆): 1.39(d,6H), 3.25-3.32(m,1H), 7.57-7.61(m,3H), 8.28-8.30(m,3H), 13.30(bs,1H)

Reference Production Examples will be described below with respect to intermediates in the production of the present compound.

### Reference Production Example 1

To a mixture of 6 ml of toluene, 2 g of 6-trifluoromethyl-3H-pyrimidin-4-one and 0.18 g of N,N-dimethylformamide, 2.9 g of thionyl chloride was slowly added dropwise at 80° C. This mixture was stirred at 80° C. for 4 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To 5 ml of water, 0.66 g of sodium cyanide and 0.14 g of 1,4-diazabicyclo[2.2.2]octane were added. To this mixture, 15 ml of dimethyl sulfoxide and the crude product were added at room temperature. This mixture was stirred at room temperature for 20 minutes. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 24 ml of ethanol, 2.1 g of sodium hydrogen carbonate and 1.7 g of hydroxylamine hydrochloride were added. This mixture was heated under reflux for 1 hour. This mixture was left standing to cool to 0° C., the crude product was added to the mixture, followed by stirring at 0° C. for 1 hour.

The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1.5 g of 6-trifluoromethylpyrimidine-4-carboxamide oxime.

### 6-trifluoromethylpyrimidine-4-carboxamide oxime

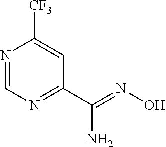

¹H-NMR (DMSO-d₆): 5.62(s,2H), 8.14(d,1H), 9.45(d,1H), 10.74(s,1H)

### Reference Production Example 2

To 12.58 g of sodium methoxide (28% methanol solution), 2.97 g of 2,2-dimethylpropionamidine hydrochloride and 4 g of 4,4,4-trifluoro-3-oxo-butanoic acid ethyl ester were added. This mixture was stirred at 80° C. for 10 hours and then heated under reflux for 6 hours. The reaction mixture was left standing to cool and then concentrated. To the residue, 10% hydrochloric acid was added, followed by extraction three times with ethyl acetate. The organic layer was dried over anhydrous magnesium sulfate and then concentrated. The residue was washed with hexane to obtain 2.4 g of 2-(1,1-dimethylethyl)-6-trifluoromethylpyrimidin-4-ol.

### 2-(1,1-dimethylethyl)-6-trifluoromethylpyrimidin-4-ol

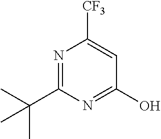

¹H-NMR (DMSO-d₆): 1.30(s,9H), 6.72(s,1H), 12.70(bs,1H)

### Reference Production Example 3

To a mixture of 10 ml of toluene, 2.4 g of 2-(1,1-dimethylethyl)-6-trifluoromethylpyrimidin-4-ol and 0.16 g of N,N-dimethylformamide, 1.6 g of thionyl chloride was slowly added dropwise at 80° C. This mixture was stirred at 80° C. for 7 hours. The reaction mixture was cooled to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To 5 ml of water, 0.59 g of sodium cyanide and 0.11 g of 1,4-diazabicyclo[2.2.2]octane were added. To this mixture, 15 ml of dimethyl sulfoxide and the crude product were added at room temperature. This mixture was stirred at room temperature for 1 hour. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 20 ml of ethanol, 1.8 g of sodium hydrogen carbonate and 1.5 g of hydroxylamine hydrochloride were added. This mixture was heated under reflux for 1 hour. After cooling this mixture to 0° C., the crude product was added, followed by stirring at 0° C. for 2 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 2.4 g of 2-(1,1-dimethylethyl)-6-trifluoromethylpyrimidine-4-carboxamide oxime.

### 2-(1,1-dimethylethyl)-6-trifluoromethylpyrimidine-4-carboxamide oxime

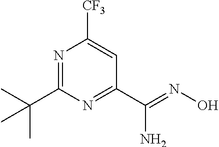

¹H-NMR: 1.45(s,9H), 5.63(bs,2H), 7.38(s,1H), 7.96(s,1H)

### Reference Production Example 4

To 30 ml of ethanol, 10 g of 4,4-difluoro-3-oxo-butanoic acid ethyl ester and 18.56 g of ammonium acetate were added. This mixture was stirred at 70° C. for 1 hour and a half. The reaction mixture was left standing to cool and then concentrated. To the residue, water was added, followed by extraction three times with ethyl acetate. The organic layer was washed with saturated brine, dried over anhydrous magnesium sulfate and then concentrated to obtain a crude product. This crude product was used in the next step without purification.

To 116 g of sodium methoxide (28% methanol solution), 10.58 g of formamide and the crude product were added. This mixture was stirred at 80° C. for 6 hours. The reaction mixture was left standing to cool and then concentrated. To the residue, 10% hydrochloric acid was added, followed by extraction five times with ethyl acetate. The organic layer was washed with saturated brine, dried over anhydrous magnesium sulfate and then concentrated. The residue was washed with hexane and tert-butyl methyl ether to obtain 2.4 g of 6-difluoromethylpyrimidin-4-ol.

### 6-difluoromethylpyrimidin-4-ol

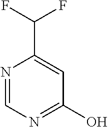

¹H-NMR (DMSO-d₆): 6.59(s,1H), 6.72(t,1H), 8.30(s,1H), 12.87 (bs, 1H)

### Reference Production Example 5

To a mixture of 20 ml of toluene, 2.4 g of 6-difluoromethylpyrimidin-4-ol and 0.24 g of N,N-dimethylformamide, 3.91 g of thionyl chloride was slowly added dropwise at 80° C. This mixture was stirred at 80° C. for 6 hours. The reaction mixture was left standing to cool to room temperature, poured into water and then extracted three times with tert-butyl methyl ether. The organic layer was washed with an aqueous saturated sodium hydrogen carbonate solution and saturated brine, dried over anhydrous magnesium sulfate and then concentrated to obtain a crude product. This crude product was used in the next step without purification.

To 5 ml of water, 0.97 g of sodium cyanide and 0.18 g of 1,4-diazabicyclo[2.2.2]octane were added. To this mixture, 15 ml of dimethyl sulfoxide and the crude product were added at room temperature. This mixture was stirred for 2 hours. The reaction mixture was poured into water and then extracted three times with tert-butyl methyl ether. The organic layer was washed with saturated brine, dried over anhydrous magnesium sulfate and then concentrated. The residue was subjected to silica gel column chromatography to obtain 2 g of 6-difluoromethylpyrimidine-4-carbonitrile. 6-difluoromethylpyrimidine-4-carbonitrile

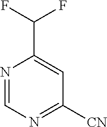

¹H-NMR: 6.33(t,1H), 7.96(s,1H), 9.49(s,1H)

### Reference Production Example 6

To 22 ml of ethanol, 2.2 g of sodium hydrogen carbonate and 1.8 g of hydroxylamine hydrochloride were added. This mixture was heated under reflux for 1 hour. After cooling this mixture to 0° C., 6-difluoromethylpyrimidine-4-carbonitrile was added. This mixture was stirred for 1 hour and a half. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 2 g of 6-difluoromethylpyrimidine-4-carboxamide oxime.

### 6-difluoromethylpyrimidine-4-carboxamide oxime

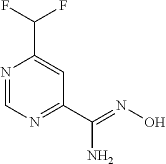

¹H-NMR (DMSO-d₆): 6.08(s,2H), 7.04(t,1H), 8.03(s,1H), 9.35(s,1H), 10.59(s,1H)

### Reference Production Example 7

To 30 ml of ethanol, 10 g of 4,4,5,5,5-pentafluoro-3-oxo-pentanoic acid ethyl ester and 13.16 g of ammonium acetate were added. This mixture was stirred at 70° C. for 4 hours and a half. This mixture was left standing to cool and then concentrated. To the residue, water was added, followed by extraction three times with ethyl acetate. The organic layer was washed with saturated brine, dried over anhydrous magnesium sulfate and then concentrated to obtain a crude product. This crude product was used in the next step without purification.

To 82.4 g of sodium methoxide (28% methanol solution), 7.7 g of formamide and the crude product were added. This mixture was heated under reflux for 8 hours. The reaction mixture was left standing to cool and then concentrated. To the residue, 10% hydrochloric acid was added, followed by extraction three times with ethyl acetate. The organic layer was washed with saturated brine, dried over anhydrous magnesium sulfate and then concentrated. The residue was washed with hexane to obtain 1.3 g of 6-(1,1,2,2,2-pentafluoroethyl)pyrimidin-4-ol.

### 6-(1,1,2,2,2-pentafluoroethyl)pyrimidin-4-ol

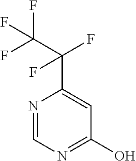

¹H-NMR (DMSO-d₆): 6.91(s,1H), 8.41(s,1H), 13.22(bs,1H)

### Reference Production Example 8

To a mixture of 10 ml of toluene, 1.3 g of 6-(1,1,2,2,2-pentafluoroethyl)pyrimidin-4-ol and 0.09 g of N,N-dimethylformamide, 1.44 g of thionyl chloride was slowly added dropwise at 80° C. This mixture was stirred at 80° C. for 6 hours. The reaction mixture was left standing to cool to room temperature, poured into water and then extracted three times with tert-butyl methyl ether. The organic layer was washed with an aqueous saturated sodium hydrogen carbonate solution and saturated brine, dried over anhydrous magnesium sulfate and then concentrated to obtain a crude product. This crude product was used in the next step without purification.

To a mixture of 2 ml of water, 0.3 g of sodium cyanide and 0.13 g of 1,4-diazabicyclo[2.2.2]octane, 7 ml of dimethyl sulfoxide and the crude product were added at 0° C. This mixture was stirred for 1 hour. The reaction mixture was poured into water and then extracted three times with tert-butyl methyl ether. The organic layer was washed with saturated brine, dried over anhydrous magnesium sulfate and then concentrated. The residue was subjected to silica gel column chromatography to obtain 1 g of 6-(1,1,2,2,2-pentafluoroethyl)pyrimidine-4-carbonitrile.

### 6-(1,1,2,2,2-pentafluoroethyl)pyrimidine-4-carbonitrile

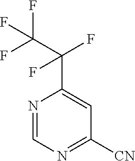

¹H-NMR: 8.02(s,1H), 9.57(s,1H)

### Reference Production Example 9

To 9 ml of ethanol, 0.75 g of sodium hydrogen carbonate and 0.62 g of hydroxylamine hydrochloride were added. This mixture was heated under reflux for 1 hour. After cooling this mixture to 0° C., 6-(1,1,2,2,2-pentafluoroethyl)pyrimidine-4-carbonitrile was added, followed by stirring for 2 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 0.85 g of 6-(1,1,2,2,2-pentafluoromethyl)pyrimidine-4-carboxamide oxime.

### 6-(1,1,2,2,2-pentafluoroethyl)pyrimidine-4-carboxamide oxime

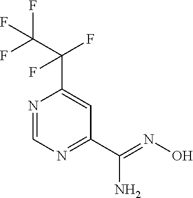

¹H-NMR (DMSO-d₆): 6.17(s,2H), 8.17(s,1H), 9.48(s,1H), 10.75(s,1H)

### Reference Production Example 10

To 40 g of sodium methoxide (28% methanol solution), 5 g of cyclopropanecarboxamidine hydrochloride and 7.63 g of 4,4,4-trifluoro-3-oxo-butanoic acid ethyl ester were added. This mixture was stirred at 80° C. for 10 hours and then heated under reflux for 12 hours. The reaction mixture was left standing to cool and then concentrated. To the residue, 10% hydrochloric acid was added and then a precipitated crystal was collected by filtration. This crystal was washed with water and then dissolved in ethyl acetate. The ethyl acetate solution was dried over anhydrous magnesium sulfate and then concentrated. The residue was washed with hexane to obtain 4.6 g of 2-cyclopropyl-6-trifluoromethylpyrimidin-4-ol.

### 2-cyclopropyl-6-trifluoromethylpyrimidin-4-ol

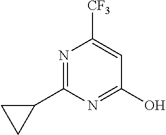

¹H-NMR (DMSO-d₆): 1.02-1.14(m,4H), 1.96-2.03(m,1H), 6.58(s,1H), 13.21(bs,1H)

### Reference Production Example 11

To a mixture of 30 ml of toluene, 3 g of 2-cyclopropyl-6-trifluoromethylpyrimidin-4-ol and 0.22 g of N,N-dimethylformamide, 3.5 g of thionyl chloride was slowly added dropwise at 80° C. This mixture was stirred at 80° C. for 4 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 5 ml of water, 0.87 g of sodium cyanide and 0.33 g of 1,4-diazabicyclo[2.2.2]octane, 7 ml of dimethyl sulfoxide and the crude product were added at 0° C. This mixture was stirred for 2 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 28 ml of ethanol, 2.36 g of sodium hydrogen carbonate and 1.96 g of hydroxylamine hydrochloride were added. This mixture was heated under reflux for 1 hour. After cooling this mixture to 0° C., the crude product was added to this mixture, followed by stirring for 2 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 3.15 g of 2-cyclopropyl-6-trifluoromethylpyrimidine-4-carboxamide oxime.

### 2-cyclopropyl-6-trifluoromethylpyrimidine-4-carboxamide oxime

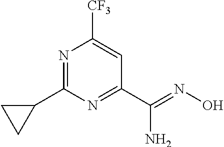

¹H-NMR (DMSO-d₆): 1.14-1.22(m,4H), 2.30-2.37 (m,1H), 6.07(s,2H), 7.84(s,1H), 10.63(s,1H)

### Reference Production Example 12

To 21 g of sodium methoxide (28% methanol solution), 4 g of 2-methylpropionamidine hydrochloride and 4 g of 4,4,4-trifluoro-3-oxo-butanoic acid ethyl ester were added. This mixture was heated under reflux for 17 hours. The reaction mixture was left standing to cool and then concentrated. To the residue, 10% hydrochloric acid was added. A precipitated crystal was collected by filtration. This crystal was washed with water. This crystal was dissolved in ethyl acetate. This ethyl acetate solution was dried over anhydrous magnesium sulfate and then concentrated. The residue was washed with hexane to obtain 2.8 g of 2-isopropyl-6-trifluoromethylpyrimidin-4-ol.

### 2-isopropyl-6-trifluoromethylpyrimidin-4-ol

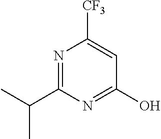

¹H-NMR: 1.40(d,6H), 2.98-3.05(m,1H), 6.71(s,1H), 13.03 (bs, 1H)

### Reference Production Example 13

To a mixture of 20 ml of toluene, 2 g of 2-isopropyl-6-trifluoromethylpyrimidin-4-o1 and 0.14 g of N,N-dimethylformamide, 2.31 g of thionyl chloride was slowly added dropwise at 80° C. This mixture was stirred at 80° C. for 3 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 3 ml of water, 0.57 g of sodium cyanide and 0.11 g of 1,4-diazabicyclo[2.2.2]octane, 9 ml of dimethyl sulfoxide and the crude product were added at 0° C. This mixture was stirred for 40 minutes. The reaction mixture was poured into water and then extracted three times with tent-butyl methyl ether. The organic layer was washed with saturated brine, dried over anhydrous magnesium sulfate and then concentrated. The residue was subjected to silica gel column chromatography to obtain 1.9 g of 2-isopropyl-6-trifluoromethylpyrimidine-4-carbonitrile.

### 2-isopropyl-6-trifluoromethylpyrimidine-4-carbonitrile

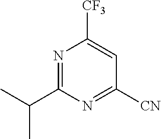

¹ H-NMR: 1.40(d,6H), 3.35-3.42(m,1H), 7.75(s,1H)

### Reference Production Example 14

To 17 ml of ethanol, 1.48 g of sodium hydrogen carbonate and 1.23 g of hydroxylamine hydrochloride were added. This mixture was heated under reflux for 1 hour. After cooling the mixture to 0° C., 1.9 g of 2-isopropyl-6-trifluoromethylpyrimidine-4-carbonitrile was added to this mixture, followed by stirring at 0° C. for 30 minutes and at room temperature for 3 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1.9 g of 2-isopropyl-6-trifluoromethylpyrimidine-4-carboxamide oxime.

### 2-isopropyl-6-trifluoromethylpyrimidine-4-carboxamide oxime

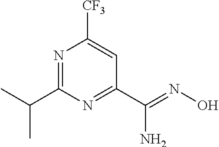

¹H-NMR: 1.40(d,6H), 3.30-3.38(m,1H), 5.73(s,2H), 8.01(s,1H), 8.80(bs,1H)

### Reference Production Example 15

To 7 ml of ethanol, 2.31 g of sodium acetate, 4 g of formamidine hydrochloride and 2 g of 4,4,5,5,6,6,6-heptafluoro-3-oxo-hexanoic acid ethyl ester were added. This mixture was stirred at 80° C. for 1 hour and then heated under reflux for 20 hours. The reaction mixture was left standing to cool and then concentrated. To the residue, 10% hydrochloric acid was added. A precipitated crystal was collected by filtration. This crystal was washed with water and then dissolved in ethyl acetate. This ethyl acetate solution was dried over anhydrous magnesium sulfate and then concentrated. The residue was washed with hexane to obtain 1 g of 6-(1,1,2,2,3,3,3-heptafluoropropyl)pyrimidin-4-ol.

### 6-(1,1,2,2,3,3,3-heptafluoropropyl)pyrimidin-4-ol

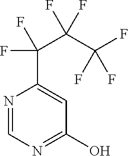

¹H-NMR (DMSO-d₆): 6.90(s,1H), 8.41(s,1H), 13.22(bs,1H)

### Reference Production Example 16

To a mixture of 10 ml of toluene, 1 g of 6-(1,1,2,2,3,3,3-heptafluoropropyl)pyrimidin-4-ol and 0.06 g of N,N-dimethylformamide, 1.35 g of thionyl chloride was slowly added dropwise at 80° C. This mixture was stirred at 80° C. for 6 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 2 ml of water, 0.23 g of sodium cyanide and 0.04 g of 1,4-diazabioyclo[2.2.2]octane, 6 ml of dimethyl sulfoxide and the crude product were added at 0° C. This mixture was stirred for 2 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 8 ml of ethanol, 0.64 g of sodium hydrogen carbonate and 0.53 g of hydroxylamine hydrochloride were added. This mixture was heated under reflux for 1 hour. After cooling this mixture to 0° C., the crude product was added to this mixture, followed by stirring for 1 hour. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 0.51 g of 6-(1,1,2,2,3,3,3-heptafluoropropyl)pyrimidine-4-carboxamide oxime. 6-(1,1,2,2,3,3,3-heptafluoropropyl)pyrimidine-4-carboxamide oxime

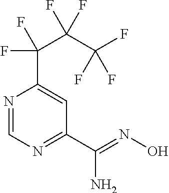

¹H-NMR: 5.64(s,2H), 7.39(s,1H), 8.24(s,1H), 9.36(s,1H)

### Reference Production Example 17

To 15 ml of N,N-dimethylformamide, 1.5 g of 6-trifluoromethylpyrimidin-4-ol and 1.79 g of N-bromosuccinimide were added. This mixture was stirred at room temperature for 12 hours and then stirred at 50° C. for 4 hours. The reaction mixture was poured into water. A precipitated crystal was collected by filtration. This crystal was washed with water to obtain 1.6 g of 6-trifluoromethyl-5-bromopyrimidin-4-ol.

### 6-trifluoromethyl-5-bromopyrimidin-4-ol

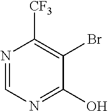

¹H-NMR (DMSO-d₆): 8.39(s,1H), 13.61(bs,1H)

### Reference Production Example 18

To a mixture of 16 ml of toluene, 1.6 g of 6-trifluoromethyl-5-bromopyrimidin-4-ol and 0.1 g of N,N-dimethylformamide, 1.57 g of thionyl chloride was slowly added dropwise at 80° C. This mixture was stirred at 80° C. for 1 hour and a half. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 3 ml of water, 0.39 g of sodium cyanide and 0.07 g of 1,4-diazabicyclo[2.2.2]octane, 9 ml of dimethyl sulfoxide and the crude product were added at 0° C. This mixture was stirred at room temperature for 30 minutes. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 20 ml of ethanol, 1.11 g of sodium hydrogen carbonate and 0.91 g of hydroxylamine hydrochloride were added. This mixture was heated under reflux for 1 hour. After cooling the mixture to 0° C., the crude product was added to this mixture, followed by stirring for 3 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1.5 g of 6-trifluoromethyl-5-bromopyrimidine-4-carboxamide oxime.

### 6-trifluoromethyl-5-bromopyrimidine-4-carboxamide oxime

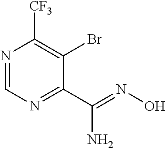

¹H-NMR (DMSO-d₆): 6.09(s,2H), 9.39(s,1H), 10.13(s,1H)

### Reference Production Example 19

To 40 ml of 1,4-dioxane, 3 g of 4,6-dichloropyrimidine, 5.57 g of potassium carbonate, 0.7 g of tetrakis(triphenylphosphine)palladium and 4.2 g of 4-trifluoromethylphenylboronic acid were added. This mixture was stirred at 100° C. for 6 hours. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 1.6 g of 4-chloro-6-(4-trifluoromethylphenyl)pyrimidine.

### 4-chloro-6-(4-trifluoromethylphenyl)pyrimidine

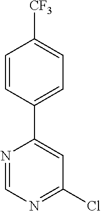

¹H-NMR: 7.74-7.77(m,3H), 8.15-8.18(m,2H), 9.04(s,1H)

### Reference Production Example 20

To a mixture of 3 ml of water, 0.36 g of sodium cyanide and 0.07 g of 1,4-diazabicyclo[2.2.2]octane, 9 ml of dimethyl sulfoxide and 1.6 g of 4-chloro-6-(4-trifluoromethylphenyl)pyrimidine were added at 0° C. This mixture was stirred for 2 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 12 ml of ethanol, 1 g of sodium hydrogen carbonate and 0.86 g of hydroxylamine hydrochloride were added. This mixture was heated under reflux for 1 hour. After cooling this mixture to 0° C., the crude product was added to this mixture, followed by stirring for 2 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1 g of 6-(4-trifluoromethylphenyl)pyrimidine-4-carboxamide oxime.

### 6-(4-trifluoromethylphenyl)pyrimidine-4-carboxamide oxime

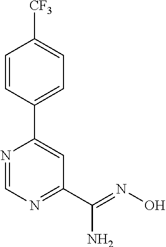

¹H-NMR (DMSO-d₆): 6.06(s,2H), 7.93(d,2H), 8.36(s,1H), 8.39(d,2H), 9.33(s,1H), 10.45(s,1H)

### Reference Production Example 21

A mixture of 12.6 g of sodium methoxide (28% methanol solution), 2.86 g of 2,2,2-trifluoroacetoamidine and 4 g of 4,4,4-trifluoro-3-oxo-butanoic acid ethyl ester was heated under reflux for 20 hours. The reaction mixture was left standing to cool and then concentrated. To the residue, 10% hydrochloric acid was added. A precipitated crystal was collected by filtration. This crystal was washed with water and then dissolved in ethyl acetate. This ethyl acetate solution was dried over anhydrous magnesium sulfate and then concentrated. The residue was washed with hexane to obtain 1.2 g of 2,6-bis(trifluoromethyl)pyrimidin-4-ol.

### 2,6-bis(trifluoromethyl)pyrimidin-4-ol

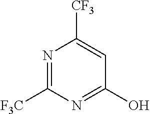

¹H-NMR (DMSO-d₆): 7.46(s,1H)

### Reference Production Example 22

To a mixture of 10 ml of toluene, 1 g of 2,6-bis(trifluoromethyl)pyrimidin-4-ol and 0.06 g of N,N-dimethylformamide, 1 g of thionyl chloride was slowly added dropwise at 80° C. This mixture was stirred at 80° C. for 3 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 2 ml of water, 0.25 g of sodium cyanide and 0.05 g of 1,4-diazabicyclo[2.2.2]octane, 6 ml of dimethyl sulfoxide and the crude product were added at 0° C. This mixture was stirred for 1 hour. The reaction mixture was poured into water and then extracted three times with tent-butyl methyl ether. The organic layer was washed with saturated brine, dried over anhydrous magnesium sulfate and then concentrated to obtain a crude product. This crude product was used in the next step without purification.

To 4 ml of ethanol, 0.39 g of sodium hydrogen carbonate and 0.29 g of hydroxylamine hydrochloride were added, and the mixture was heated under reflux for 1 hour. After cooling this mixture to 0° C., the crude product was added to this mixture, followed by stirring for 2 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1.5 g of 2,6-bis(trifluoromethyl)pyrimidine-4-carboxamide oxime.

### 2,6-bis(trifluoromethyl)pyrimidine-4-carboxamide oxime

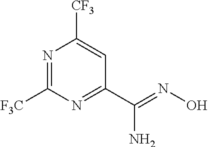

¹H-NMR (DMSO-d₆): 6.19(s,2H), 8.35(s,1H), 11.04(s,1H)

### Reference Production Example 23

To 30 ml of 1,4-dioxane, 3 g of 4,6-dichloropyrimidine, 5.57 g of potassium carbonate, 0.7 g of tetrakis(triphenylphosphine)palladium and 4.2 g of 3-trifluoromethylphenylboronic acid were added. This mixture was stirred at 90° C. for 8 hours. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 1.5 g of 4-chloro-6-(3-trifluoromethylphenyl)pyrimidine.

### 4-chloro-6-(3-trifluoromethylphenyl)pyrimidine

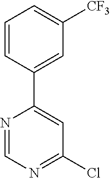

¹H-NMR: 7.67(t,1H), 7.80-7.82(m,2H), 8.26(d,1H), 8.37(s,1H), 9.08(s,1H)

### Reference Production Example 24

0.7 g of sodium hydride (60% in oil) was suspended in 30 ml of toluene and 1.34 g of 2,2,2-trifluoroethanol was added dropwise. After stirring the mixture for 10 minutes, 2 g of 4,6-dichloropyrimidine was added to this mixture at 0° C. This mixture was stirred at 0° C. for 2 hours and then stirred at room temperature for 3 hours. The reaction mixture was poured into an aqueous saturated ammonium chloride solution and then extracted three times with tert-butyl methyl ether. The organic layer was washed with saturated brine, dried over anhydrous magnesium sulfate and then concentrated to obtain a crude product. This crude product was used in the next step without purification.

To a mixture of 2 ml of water, 0.79 g of sodium cyanide and 0.16 g of 1,4-diazabicyclo[2.2.2]octane, 9 ml of dimethyl sulfoxide and the crude product were added at 0° C. This mixture was stirred for 3 hours. The reaction mixture was poured into water and then extracted three times with tert-butyl methyl ether. The organic layer was washed with saturated brine, dried over anhydrous magnesium sulfate and then concentrated. The residue was subjected to silica gel column chromatography to obtain 0.5 g of 4-(2,2,2-trifluoroethoxy)pyrimidine-6-carbonitrile.

### 4-(2,2,2-trifluoroethoxy)pyrimidine-6-carbonitrile

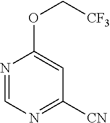

¹H-NMR: 4.89(q,2H), 7.28(d,1H), 8.90(d,1H)

### Reference Production Example 25

To 25 ml of ethanol, 2 g of cyclopropanecarboxamidine hydrochloride and 2.32 g of sodium hydrogen carbonate were added. This mixture was heated under reflux for 1 hour. After the mixture was left standing to cool, 3.2 g of 4,4,5,5,5-pentafluoro-3-oxo-pentanoic acid ethyl ester was added to this mixture, followed by stirring at 60° C. for 4 hours, and the mixture was heated under reflux for 20 hours. The reaction mixture was left standing to cool and then concentrated. 10% hydrochloric acid was added to the residue, followed by extraction three times with ethyl acetate. The organic layer was washed with saturated brine, dried over anhydrous magnesium sulfate and then concentrated. The residue was washed with hexane to obtain 1.1 g of 2-cyclopropyl-6-(1,1,2,2,2-pentafluoroethyl)pyrimidin-4-ol.

### 2-cyclopropyl-6-(1,1,2,2,2-pentafluoroethyl)pyrimidin-4-ol

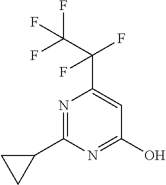

¹H-NMR (DMSO-d₆): 0.98-1.02(m,2H), 1.09-1.14(m,2H), 1.96-2.01(m,1H), 6.62(s,1H), 13.29(bs,1H)

### Reference Production Example 26

To a mixture of 10 ml of toluene, 1.1 g of 2-cyclopropyl-6-(1,1,2,2,2-pentafluoroethyl)pyrimidin-4-ol, and 0.07 g of N,N-dimethylformamide, 1 g of thionyl chloride was slowly added dropwise at 80° C. This mixture was stirred at 80° C. for 4 hours. The reaction mixture was left standing to cool to room temperature, poured into water and then extracted three times with tert-butyl methyl ether. The organic layer was washed with an aqueous saturated sodium hydrogen carbonate solution and saturated brine, dried over anhydrous magnesium sulfate and then concentrated to obtain a crude product. This crude product was used in the next step without purification.

To a mixture of 2 ml of water, 0.26 g of sodium cyanide and 0.05 g of 1,4-diazabicyclo[2.2.2]octane, 6 ml of dimethyl sulfoxide and the crude product were added at 0° C. This mixture was stirred at for 2 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 8 ml of ethanol, 0.73 g of sodium hydrogen carbonate and 0.6 g of hydroxylamine hydrochloride were added. This mixture was heated under reflux for 1 hour. After cooling this mixture to 0° C., the crude product was added to the mixture, followed by stirring for 1 hour. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1.1 g of 2-cyclopropyl-6-(1,1,2,2,2-pentafluoroethyl)pyrimidine-4-carboxamide oxime.

### 2-cyclopropyl-6-(1,1,2,2,2-pentafluoroethyl)pyrimidine-4-carboxamide oxime

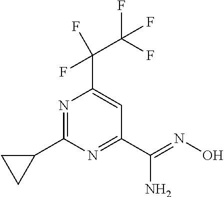

¹H-NMR (DMSO-d₆): 1.16-1.19(m,4H), 2.30-2.37(m,1H), 6.10(s,2H), 7.87(s,1H), 10.65(s,1H)

### Reference Production Example 27

To 30 ml of 1,4-dioxane, 3 g of 4,6-dichloropyrimidine, 5.57 g of potassium carbonate, 0.7 g of tetrakis(triphenylphosphine)palladium and 4.2 g of 2-trifluoromethylphenylboronic acid were added. This mixture was stirred at 90° C. for 8 hours. After the reaction mixture was left standing to cool to room temperature, the reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 2.3 g of 4-chloro-6-(2-trifluoromethylphenyl)pyrimidine.

### 4-chloro-6-(2-trifluoromethylphenyl)pyrimidine

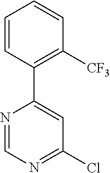

¹H-NMR: 7.49-7.53(m,2H), 7.61-7.70(m,2H), 7.83(d,1H), 9.08(s,1H)

### Reference Production Example 28

To a mixture of 3 ml of water, 0.52 g of sodium cyanide and 0.11 g of 1,4-diazabicyclo[2.2.2]octane, 12 ml of dimethyl sulfoxide and 2.3 g of 4-chloro-6-(2-trifluoromethylphenyl)pyrimidine were added at 0° C. This mixture was stirred at room temperature for 3 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 15 ml of ethanol, 1.5 g of sodium hydrogen carbonate and 1.24 g of hydroxylamine hydrochloride were added. This mixture was heated under reflux for 1 hour. After cooling this mixture to 0° C., the crude product was added to this mixture, followed by stirring for 6 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 2.3 g of 6-(2-trifluoromethylphenyl)pyrimidine-4-carboxamide oxime.

### 6-(2-trifluoromethylphenyl)pyrimidine-4-carboxamide oxime

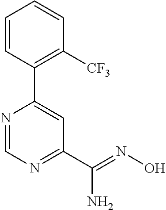

¹H-NMR (DMSO-d₆): 6.06(3,2H), 7.66(d,1H), 7.76(t,1H), 7.83(t,1H), 7.91-7.93(m,2H), 9.30(s,1H), 10.48(s,1H)

### Reference Production Example 29

To 12.6 g of sodium methoxide (28% methanol solution), 3.1 g of acetoamidine hydrochloride and 3 g of 4,4,4-trifluoro-3-oxo-butanoic acid ethyl ester were added. This mixture was stirred at 80° C. for 20 hours. The reaction mixture was left standing to cool and then concentrated. To the residue, 10% hydrochloric acid was added, followed by extraction three times with ethyl acetate. The organic layer was dried over anhydrous magnesium sulfate and then concentrated. The residue was washed with hexane to obtain 2.4 g of 2-methyl-6-trifluoromethylpyrimidin-4-ol.

### 2-methyl-6-trifluoromethylpyrimidin-4-ol

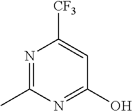

¹H-NMR (DMSO-d₆): 2.35(s,3H), 6.68(s,1H), 13.02(bs,1H)

### Reference Production Example 30

To a mixture of 20 ml of toluene, 2.2 g of 2-methyl-6-trifluoromethylpyrimidin-4-ol and 0.19 g of N,N-dimethylformamide, 2.94 g of thionyl chloride was slowly added dropwise at 80° C. This mixture was stirred at 80° C. for 2 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 3 ml of water, 0.73 g of sodium cyanide and 0.15 g of 1,4-diazabicyclo[2.2.2]octane, 9 ml of dimethyl sulfoxide and the crude product were added at 0° C. This mixture was stirred for 2 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 20 ml of ethanol, 2.1 g of sodium hydrogen carbonate and 1.7 g of hydroxylamine hydrochloride were added. This mixture was heated under reflux for 1 hour. After cooling this mixture to 0° C., the crude product was added to this mixture, followed by stirring for 3 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1.8 g of 2-methyl-6-trifluoromethylpyrimidine-4-carboxamide oxime.

### 2-methyl-6-trifluoromethylpyrimidine-4-carboxamide oxime

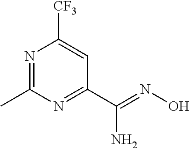

¹H-NMR (DMSO-d₆): 2.8(s,3H), 5.53(s,2H), 8.02(s,1H), 10.13(s,1H)

### Reference Production Example 31

To 12.6 g of sodium methoxide (28% methanol solution), 5.61 g of O-methylisouronium sulfate and 3 g of 4,4,4-trifluoro-3-oxo-butanoic acid ethyl ester were added. This mixture was stirred at 80° C. for 20 hours. The reaction mixture was left standing to cool and then concentrated. To the residue, 10% hydrochloric acid was added. A precipitated crystal was collected by filtration and then washed with water to obtain 1.5 g of 2-methoxy-6-trifluoromethylpyrimidin-4-ol.

### 2-methoxy-6-trifluoromethylpyrimidin-4-ol

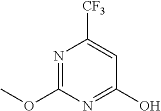

¹H-NMR (DMSO-d₆): 3.93(s,3H), 6.50(s,1H), 13.00(bs,1H)

### Reference Production Example 32

To a mixture of 14 ml of toluene, 1.4 g of 2-methoxy-6-trifluoromethylpyrimidin-4-ol and 0.11 g of N,N-dimethylformamide, 1.72 g of thionyl chloride was slowly added dropwise at 80° C. This mixture was stirred at 80° C. for 4 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 2 ml of water, 0.42 g of sodium cyanide and 0.09 g of 1,4-diazabicyclo[2.2.2]octane, 6 ml of dimethyl sulfoxide and the crude product were added at 0° C. This mixture was stirred for 1 hour. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 14 ml of ethanol, 1.22 g of sodium hydrogen carbonate and 1 g of hydroxylamine hydrochloride were added. This mixture was heated under reflux for 1 hour. After cooling this mixture to 0° C., the crude product was added to this mixture, followed by stirring for 4 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 0.7 g of 2-methoxy-6-trifluoromethylpyrimidine-4-carboxamide oxime.

### 2-methoxy-6-trifluoromethylpyrimidine-4-carboxamide oxime

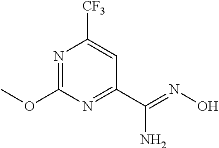

¹H-NMR (DMSO-d₆): 4.06(s,3H), 6.10(s,2H), 7.73(s,1H), 10.72(s,1H)

### Reference Production Example 33

To 16.8 g of sodium methoxide (28% methanol solution), 4.1 g of benzamidine hydrochloride and 4 g of 4,4,4-trifluoro-3-oxo-butanoic acid ethyl ester were added. This mixture was stirred at 100° C. for 20 hours. The reaction mixture was left standing to cool and then concentrated. To the residue, 10% hydrochloric acid was added. A precipitated crystal was collected by filtration and then washed with water to obtain 1.8 g of 2-phenyl-6-trifluoromethylpyrimidin-4-ol.

### 2-phenyl-6-trifluoromethylpyrimidin-4-ol

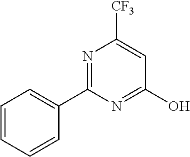

¹H-NMR (DMSO-d₆): 6.86(s,1H), 7.57(t,2H), 7.63(d,1H), 8.13(d,2H), 13.32(bs,1H)

### Reference Production Example 34

To a mixture of 17 ml of toluene, 1.7 g of 2-phenyl-6-trifluoromethylpyrimidin-4-ol and 0.25 g of N,N-dimethylformamide, 1.68 g of thionyl chloride was added slowly added dropwise at 80° C. This mixture was stirred at 80° C. for 4 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 2 ml of water, 0.42 g of sodium cyanide and 0.09 g of 1,4-diazabicyclo[2.2.2]octane, 8 ml of dimethyl sulfoxide and the crude product were added at 0° C. This mixture was stirred for 40 minutes. The reaction mixture was poured into water and then extracted three times with tert-butyl methyl ether. The organic layer was washed with saturated brine, dried over anhydrous magnesium sulfate and then concentrated. The residue was subjected to silica gel column chromatography to obtain 1.6 g of 2-phenyl-6-trifluoromethylpyrimidine-4-carbonitrile.

### 2-phenyl-6-trifluoromethylpyrimidine-4-carbonitrile

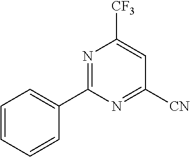

¹H-NMR: 7.53-7.61(m,3H), 7.78(s,1H), 8.51-8.54(m,2H)

### Reference Production Example 35

To 13 ml of ethanol, 1.1 g of sodium hydrogen carbonate and 0.89 g of hydroxylamine hydrochloride were added. This mixture was heated under reflux for 1 hour. After cooling this mixture to 0° C., to this mixture, 1.6 g of 2-phenyl-6-trifluoromethylpyrimidine-4-carbonitrile was added, followed by stirring at 0° C. for 30 minutes and further stirring at room temperature for 2 hours. The reaction mixture was concentrated and water was added to the residue, followed by extraction three times with ethyl acetate. The organic layer was washed with saturated brine, dried over anhydrous magnesium sulfate and then concentrated. The residue was subjected to silica gel column chromatography to obtain 1.6 g of 2-phenyl-6-trifluoromethylpyrimidine-4-carboxamide oxime.

### 2-phenyl-6-trifluoromethylpyrimidine-4-carboxamide oxime

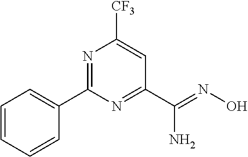

¹H-NMR (DMSO-d₆): 6.38(s,2H), 7.57-7.63(m,3H), 8.02(s,1H), 8.61-8.64(m,2H), 10.73(s,1H)

### Reference Production Example 36

To a mixture of 20 ml of toluene, 5 g of 2-methylthio-6-trifluoromethylpyrimidin-4-ol and 0.37 g of N,N-dimethylformamide, 5.7 g of thionyl chloride was slowly added dropwise at 80° C. This mixture was stirred at 80° C. for 1 hour. The reaction mixture was left standing to cool to room temperature, poured into water and then extracted three times with tent-butyl methyl ether. The organic layer was washed in turn with an aqueous saturated sodium hydrogen carbonate solution and saturated brine, dried over anhydrous magnesium sulfate and then concentrated to obtain a crude product. This crude product was used in the next step without purification.

To a mixture of 4 ml of water, 1.4 g of sodium cyanide and 0.29 g of 1,4-diazabicyclo[2.2.2]octane, 20 ml of dimethyl sulfoxide and the crude product were added at 0° C. This mixture was stirred for 1 hour. The reaction mixture was poured into water and then extracted three times with tert-butyl methyl ether. The organic layer was washed with saturated brine, dried over anhydrous magnesium sulfate and then concentrated. The residue was subjected to silica gel column chromatography to obtain 3.2 g of 2-methylthio-6-trifluoromethylpyrimidine-4-carbonitrile.

### 2-methylthio-6-trifluoromethylpyrimidine-4-carbonitrile

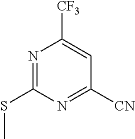

¹H-NMR: 2.63(s,3H), 7.53(s,1H)

### Reference Production Example 37

To 17 ml of ethanol, 0.77 g of sodium hydrogen carbonate and 0.63 g of hydroxylamine hydrochloride were added. This mixture was heated under reflux for 1 hour. After cooling this mixture to 0° C., 1 g of 2-methylthio-6-trifluoromethylpyrimidine-4-carbonitrile was added to this mixture, followed by stirring for 30 minutes and further stirring at room temperature for 2 hours. The reaction mixture was concentrated. To the residue, water was added, followed by extraction three times with ethyl acetate. The organic layer was washed with saturated brine, dried over anhydrous magnesium sulfate and then concentrated. The residue was subjected to silica gel column chromatography to obtain 1 g of 2-methylthio-6-trifluoromethylpyrimidine-4-carboxamide oxime.

### 2-methylthio-6-trifluoromethylpyrimidine-4-carboxamide oxime

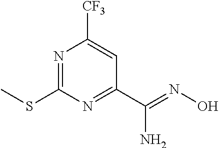

¹H-NMR (DMSO-d₆): 2.64(s,3H), 6.10(s,2H), 7.75(s,1H), 10.76(s,1H)

### Reference Production Example 38

To 50 ml of N-methyl pyrrolidone, 4 g of 4,6-dichloropyrimidine, 5.57 g of potassium carbonate and 5.46 g of 2,2,2-trifluoroethylamine hydrochloride were added. This mixture was stirred at 60° C. for 8 hours. The reaction mixture was left standing to cool, poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 3.2 g of 6-chloro-4-(2,2,2-trifluoroethylamino)pyrimidine.

### 6-chloro-4-(2,2,2-trifluoroethylamino)pyrimidine

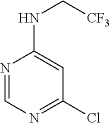

¹H-NMR: 4.11-4.20(m,2H), 5.16(bs,1H), 6.51(s,1H), 8.45(s,1H)

### Reference Production Example 39

0.12 g of sodium hydride (60% in oil) was suspended in 4 ml of N,N-dimethylformamide, and 0.5 g of 6-chloro-4-(2,2,2-trifluoroethylamino)pyrimidine was added thereto at 0° C. This mixture was stirred for 10 minutes. To the mixture, 0.44 g of iodomethane was added. This mixture was stirred at 0° C. for 20 minutes and then stirred at room temperature for 4 hours. The reaction mixture was poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 0.5 g of 6-chloro-4-[N-methyl-N-(2,2,2-trifluoroethyl)amino]pyrimidine.

### 6-chloro-4-[N-methyl-N-(2,2,2-trifluoroethyl)amino]pyrimidine

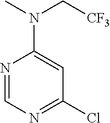

¹H-NMR: 3.15(s,3H), 4.35(q,2H), 6.55(s,1H), 8.44(s,1H)

### Reference Production Example 40

To 12 ml of N,N-dimethylformamide, 1.5 g of 6-chloro-4-[N-methyl-N-(2,2,2-trifluoroethyl)amino]pyrimidine, 1.6 g of zinc cyanide and 0.31 g of tetrakis(triphenylphosphine)palladium were added. This mixture was stirred at 90° C. for 18 hours. The reaction mixture was left standing to cool, poured into water and then extracted three times with diethylether. The organic layer was washed with saturated brine, dried over anhydrous magnesium sulfate and then concentrated. The residue was subjected to silica gel column chromatography to obtain 0.65 g of 6-[N-methyl-N-(2,2,2-trifluoroethyl)amino]pyrimidine-4-carbonitrile.

### 6-[N-methyl-N-(2,2,2-trifluoroethyl)amino]pyrimidine-4-carbonitrile

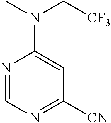

¹H-NMR: 3.20(s,3H), 4.38(bs,2H), 6.89(s,1H), 8.70(s,1H)

### Reference Production Example 41

To 4 ml of ethanol, 0.18 g of sodium hydrogen carbonate and 0.15 g of hydroxylamine hydrochloride were added. This mixture was heated under reflux for 1 hour. After cooling the reaction mixture to 0° C., 0.3 g of 6-[N-methyl-N-(2,2,2-trifluoroethyl)amino]pyrimidine-4-carbonitrile was added to this mixture. This mixture was stirred at room temperature for 4 hours. The reaction mixture was concentrated and water was added to the residue, followed by extraction three times with ethyl acetate. The organic layer was washed with saturated brine, dried over anhydrous magnesium sulfate and then concentrated. The residue was subjected to silica gel column chromatography to obtain 0.33 g of 6-[N-methyl-N-(2,2,2-trifluoroethyl)amino]pyrimidine-4-carboxamide oxime.

### 6-[N-methyl-N-(2,2,2-trifluoroethyl)amino]pyrimidine-4-carboxamide oxime

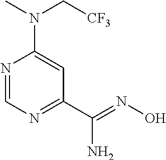

¹H-NMR: 3.17(s,3H), 4.37(q,2H), 5.57(s,2H), 6.58(s,1H), 7.10(s,1H), 8.63(s,1H)

### Reference Production Example 42

To 12 ml of 1,4-dioxane, 0.75 g of phenylboronic acid, 1.67 g of potassium carbonate, 0.13 g of dichlorobis(triphenylphosphine)palladium and 1 g of 4,6-dichloro-2-methylpyrimidine were added. This mixture was stirred at 60° C. for 3 hours and then stirred at 80° C. for 6 hours. The reaction mixture was left standing to cool to room temperature, poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 0.64 g of 4-chloro-2-methyl-6-phenylpyrimidine.

### 4-chloro-2-methyl-6-phenylpyrimidine

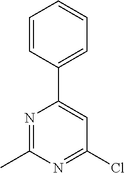

¹H-NMR: 2.78(s,3H), 7.49-7.56(m,4H), 8.04-8.07(m,2H)

### Reference Production Example 43

To 11 ml of N,N-dimethylformamide, 1 g of 4-chloro-2-methyl-6-phenylpyrimidine, 1.25 g of zinc cyanide and 0.18 g of tetrakis(triphenylphosphine)palladium were added. This mixture was stirred at 90° C. for 10 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.6 g of 2-methyl-6-phenylpyrimidine-4-carbonitrile.

### 2-methyl-6-phenylpyrimidine-4-carbonitrile

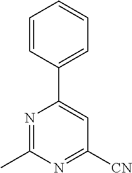

¹H-NMR: 2.85(3,3H), 7.52-7.59(m,3H), 7.84(s,1H), 8.09-8.12(m,2H)

### Reference Production Example 44

To 6 ml of ethanol, 0.51 g of sodium hydrogen carbonate and 0.42 g of hydroxylamine hydrochloride were added. This mixture was heated under reflux for 1 hour. After cooling this mixture to 0° C., 0.6 g of 2-methyl-6-phenylpyrimidine-4-carbonitrile was added to this mixture. This mixture was stirred at room temperature for 2 hours and a half. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.58 g of 2-methyl-6-phenylpyrimidine-4-carboxamide oxime.

### 2-methyl-6-phenylpyrimidine-4-carboxamide oxime

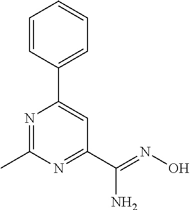

¹H-NMR (DMSO-d₆) 2.73(s,3H), 5.96(s,2H), 7.54-7.57(m,3H), 8.07 (s,1H), 8.13-8.16(m,2H), 10.33(s,1H)

### Reference Production Example 45

To 12 ml of 1,4-dioxane, 0.96 g of 3-chlorophenylboronic acid, 1.67 g of potassium carbonate, 0.13 g of dichlorobis(triphenylphosphine)palladium and 1 g of 4,6-dichloro-2-methylpyrimidine were added. This mixture was stirred at 60° C. for 3 hours and then stirred at 80° C. for 10 hours. The reaction mixture was poured into an aqueous saturated ammonium chloride solution and then extracted three times with text-butyl methyl ether. The organic layer was washed with saturated brine, dried over anhydrous magnesium sulfate and then concentrated to obtain a crude product. This crude product was used in the next step without purification.

To 5 ml of N,N-dimethylformamide, the crude product, 0.6 g of zinc cyanide and 0.09 g of tetrakis(triphenylphosphine)palladium were added. This mixture was stirred at 90° C. for 12 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.33 g of 2-methyl-6-(3-chlorophenyl)pyrimidine-4-carbonitrile.

### 2-methyl-6-(3-chlorophenyl)pyrimidine-4-carbonitrile

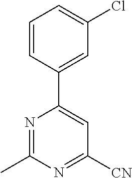

¹H-NMR: 2.86(s,3H), 7.47-7.58(m,2H), 7.81-7.84(m,1H), 7.94-7.99(m,1H), 8.14(s,1H)

### Reference Production Example 46

To 3 ml of ethanol, 0.24 g of sodium hydrogen carbonate and 0.2 g of hydroxylamine hydrochloride were added. This mixture was heated under reflux for 1 hour. After cooling this mixture to 0° C., 0.33 g of 2-methyl-6-(3-chlorophenyl)pyrimidine-4-carbonitrile was added to this mixture. This mixture was stirred at room temperature for 1 hour and a half. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.58 g of 2-methyl-6-(3-chlorophenyl)pyrimidine-4-carboxamide oxime.

### 2-methyl-6-(3-chlorophenyl)pyrimidine-4-carboxamide oxime

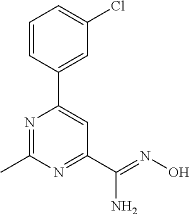

¹H-NMR (DMSO-d₆): 2.74(s,3H), 5.98(s,2H), 7.56-7.65(m,2H), 8.09-8.12(m,2H), 8.19-8.20(m,1H), 10.36(s,1H)

### Reference Production Example 47

To 12 ml of 1,4-dioxane, 0.96 g of 2-chlorophenylboronic acid, 1.67 g of potassium carbonate, 0.13 g of dichlorobis(triphenylphosphine)palladium and 1 g of 4,6-dichloro-2-methylpyrimidine were added. This mixture was stirred at 60° C. for 11 hours. The reaction mixture was poured into an aqueous saturated ammonium chloride solution and then extracted three times with tert-butyl methyl ether. The organic layer was washed with saturated brine, dried over anhydrous magnesium sulfate and then concentrated. This crude product was used in the next step without purification.

To 8 ml of N,N-dimethylformamide, the crude product, 0.8 g of zinc cyanide and 0.12 g of tetrakis(triphenylphosphine)palladium were added. This mixture was stirred at 100° C. for 10 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.22 g of 2-methyl-6-(2-chlorophenyl)pyrimidine-4-carbonitrile.

### 2-methyl-6-(2-chlorophenyl)pyrimidine-4-carbonitrile

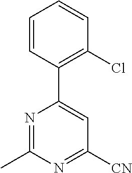

¹H-NMR: 2.87(s,3H), 7.44-7.47(m,2H), 7.52-7.54(m,1H), 7.69-7.71(m,1H), 7.91(s,1H)

### Reference Production Example 48

To 2 ml of ethanol, 0.16 g of sodium hydrogen carbonate and 0.13 g of hydroxylamine hydrochloride were added. This mixture was heated under reflux for 1 hour. After cooling this mixture to 0° C., 0.22 g of 2-methyl-6-(2-chlorophenyl)pyrimidine-4-carbonitrile was added to this mixture. This mixture was stirred at room temperature for 2 hours and a half. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.21 g of 2-methyl-6-(2-chlorophenyl)pyrimidine-4-carboxamide oxime.

### 2-methyl-6-(2-chlorophenyl)pyrimidine-4-carboxamide oxime

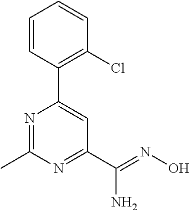

¹H-NMR (DMSO-d₆): 2.73(s,3H), 5.98(s,2H), 7.49-7.56(m,2H), 7.61-7.64(m,1H), 7.70(dd,1H), 7.93(s,1H), 10.41(s,1H)

### Reference Production Example 49

To 12 ml of 1,4-dioxane, 0.96 g of 4-chlorophenylboronic acid, 1.7 g of potassium carbonate, 0.13 g of dichlorobis(triphenylphosphine)palladium and 1 g of 4,6-dichloro-2-methylpyrimidine were added. This mixture was stirred at 60° C. for 4 hours and then stirred at 80° C. for 7 hours. The reaction mixture was left standing to cool to room temperature, poured into an aqueous saturated ammonium chloride solution and then extracted three times with tert-butyl methyl ether. The organic layer was washed with saturated brine, dried over anhydrous magnesium sulfate and then concentrated to obtain a crude product. This crude product was used in the next step without purification.

To 6 ml of N,N-dimethylformamide, the crude product, 0.67 g of zinc cyanide and 0.1 g of tetrakis(triphenylphosphine)palladium were added. This mixture was stirred at 100° C. for 14 hours. The reaction mixture was left standing to cool, poured into water, extracted three times with tert-butyl methyl ether and then washed with saturated brine. The organic layer was dried over anhydrous magnesium sulfate and then concentrated. The residue was subjected to silica gel column chromatography to obtain 0.42 g of 2-methyl-6-(4-chlorophenyl)pyrimidine-4-carbonitrile.

### 2-methyl-6-(4-chlorophenyl)pyrimidine-4-carbonitrile

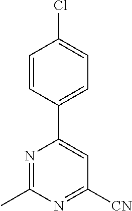

¹H-NMR: 2.84(s,3H),7.51-7.54(m,2H), 7.81(s,1H), 8.05-8.08(m,2H)

### Reference Production Example 50

To 4 ml of ethanol, 0.31 g of sodium hydrogen carbonate and 0.25 g of hydroxylamine hydrochloride were added. This mixture was heated under reflux for 1 hour. After cooling this mixture to 0° C., 0.42 g of 2-methyl-6-(4-chlorophenyl)pyrimidine-4-carbonitrile was added to this mixture. This mixture was stirred at room temperature for 1 hour and a half. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.43 g of 2-methyl-6-(4-chlorophenyl)pyrimidine-4-carboxamide oxime.

### 2-methyl-6-(4-chlorophenyl)pyrimidine-4-carboxamide oxime

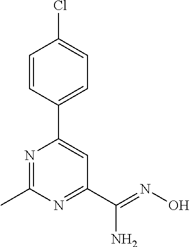

¹H-NMR (DMSO-d₆): 2.73(s,3H), 5.97(s,2H), 7.61(d,2H), 8.07(s,1H), 8.18(d,2H), 10.34(s,1H)

### Reference Production Example 51

To 12 ml of 1,4-dioxane, 0.86 g of 3-fluorophenylboronic acid, 1.7 g of potassium carbonate, 0.13 g of dichlorobis(triphenylphosphine)palladium and 1 g of 4,6-dichloro-2-methylpyrimidine were added. This mixture was stirred at 60° C. for 13 hours. The reaction mixture was poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 0.52 g of 4-chloro-2-methyl-6-(3-fluorophenyl)pyrimidine.

### 4-chloro-2-methyl-6-(3-fluorophenyl)pyrimidine

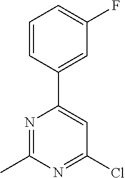

¹H-NMR: 2.78(s,3H), 7.20-7.25(m,1H), 7.45-7.51(m,1H), 7.54(s,1H), 7.80-7.84(m,2H)

### Reference Production Example 52

To 5 ml of N,N-dimethylformamide, 0.5 g of 4-chloro-2-methyl-6-(3-fluorophenyl)pyrimidine, 0.54 g of zinc cyanide and 0.08 g of tetrakis(triphenylphosphine)palladium were added. This mixture was stirred at 90° C. for 5 hours and then stirred at 100° C. for 8 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.29 g of 2-methyl-6-(3-fluorophenyl)pyrimidine-4-carbonitrile.

### 2-methyl-6-(3-fluorophenyl)pyrimidine-4-carbonitrile

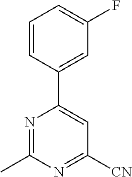

¹H-NMR: 2.86(s,3H), 7.24-7.30(m,1H), 7.49-7.55(m,1H), 7.82(s,1H), 7.84-7.89(m,2H)

### Reference Production Example 53

To 3 ml of ethanol, 0.23 g of sodium hydrogen carbonate and 0.19 g of hydroxylamine hydrochloride were added. This mixture was heated under reflux for 1 hour. After cooling this mixture to 0° C., 0.29 g of 2-methyl-6-(3-fluorophenyl)pyrimidine-4-carbonitrile was added to this mixture. This mixture was stirred at room temperature for 30 minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.29 g of 2-methyl-6-(3-fluorophenyl)pyrimidine-4-carboxamide oxime.

### 2-methyl-6-(3-fluorophenyl)pyrimidine-4-carboxamide oxime

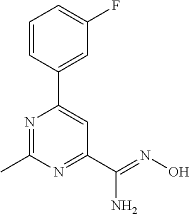

¹H-NMR (DMSO-d₆): 2.74(s,3H), 5.97(s,2H), 7.41(td,1H), 7.60(td,1H), 7.94-8.01(m,2H), 8.09(s,1H), 10.36(s,1H)

### Reference Production Example 54

To 12 ml of 1,4-dioxane, 0.86 g of 2-fluorophenylboronic acid, 1.7 g of potassium carbonate, 0.13 g of dichlorobis(triphenylphosphine)palladium and 1 g of 4,6-dichloro-2-methylpyrimidine were added. This mixture was stirred at 60° C. for 11 hours. The reaction mixture was poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 0.64 g of 4-chloro-2-methyl-6-(2-fluorophenyl)pyrimidine.

### 4-chloro-2-methyl-6-(2-fluorophenyl)pyrimidine

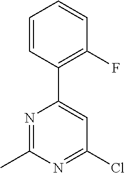

¹H-NMR: 2.78(s,3H), 7.19(dd,1H), 7.30(t,1H), 7.46-7.51(m,1H), 7.70(s,1H), 8.15-8.18(m,1H)

### Reference Production Example 55

To 6 ml of N,N-dimethylformamide, 0.64 g of 4-chloro-2-methyl-6-(2-fluorophenyl)pyrimidine, 0.34 g of zinc cyanide and 0.1 g of tetrakis(triphenylphosphine)palladium were added. This mixture was stirred at 90° C. for 4 hours and then stirred at 100° C. for 12 hours. The reaction mixture was left standing to cool, poured into water and then extracted three times with tert-butyl methyl ether. The organic layer was washed with saturated brine, dried over anhydrous magnesium sulfate and then concentrated to obtain a crude product. This crude product was used in the next step without purification.

To 3 ml of ethanol, 0.17 g of sodium hydrogen carbonate and 0.14 g of hydroxylamine hydrochloride were added. This mixture was heated under reflux for 1 hour. After cooling this mixture to 0° C., the crude product was added to this mixture. This mixture was stirred at room temperature for 2 hours. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.28 g of 2-methyl-6-(2-fluorophenyl)pyrimidine-4-carboxamide oxime.

### 2-methyl-6-(2-fluorophenyl)pyrimidine-4-carboxamide oxime

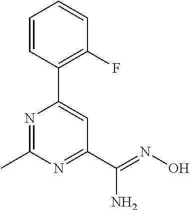

¹H-NMR (DMSO-d₆): 2.74(s,3H), 5.97(5,2H), 7.36-7.44(m,2H), 7.57-7.63(m,1H), 8.09(s,1H), 8.11-8.17(m,1H), 10.42(s,1H)

### Reference Production Example 56

To 12 ml of 1,4-dioxane, 0.83 g of 4-methylphenylboronic acid, 1.7 g of potassium carbonate, 0.13 g of dichlorobis(triphenylphosphine)palladium and 1 g of 4,6-dichloro-2-methylpyrimidine were added. This mixture was stirred at 60° C. for 12 hours. The reaction mixture was left standing to cool to room temperature, poured into an aqueous saturated ammonium chloride solution and then extracted three times with tert-butyl methyl ether. The organic layer was washed with saturated brine, dried over anhydrous magnesium sulfate and then concentrated. This crude product was used in the next step without purification.

To 8 ml of N,N-dimethylformamide, the crude product, 0.81 g of zinc cyanide and 0.12 g of tetrakis(triphenylphosphine)palladium were added. This mixture was stirred at 90° C. for 8 hours and then stirred at 100° C. for 4 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.29 g of 2-methyl-6-(4-methylphenyl)pyrimidine-4-carbonitrile.

### 2-methyl-6-(4-methylphenyl)pyrimidine-4-carbonitrile

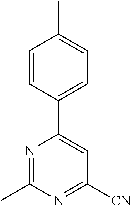

¹H-NMR: 2.45(s,3H), 2.83(s,3H), 7.34(d,2H), 7.80(s,1H), 8.00(d,2H)

### Reference Production Example 57

To 3 ml of ethanol, 0.23 g of sodium hydrogen carbonate and 0.19 g of hydroxylamine hydrochloride were added. This mixture was heated under reflux for 1 hour. After cooling this mixture to 0° C., 0.29 g of 2-methyl-6-(4-methylphenyl)pyrimidine-4-carbonitrile was added to this mixture. This mixture was stirred at room temperature for 30 minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.29 g of 2-methyl-6-(4-methylphenyl)pyrimidine-4-carboxamide oxime.

### 2-methyl-6-(4-methylphenyl)pyrimidine-4-carboxamide oxime

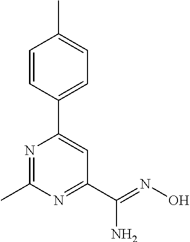

¹H-NMR (DMSO-d₆): 2.39(s,3H), 2.71(s,3H), 5.94(s,2H), 7.36(d,2H), 8.03(s,1H), 8.05(d,2H), 10.30(s,1H)

### Reference Production Example 58

To 12 ml of 1,4-dioxane, 0.83 g of 3-methylphenylboronic acid, 1.7 g of potassium carbonate, 0.13 g of dichlorobis(triphenylphosphine)palladium and 1 g of 4,6-dichloro-2-methylpyrimidine were added. This mixture was stirred at 60° C. for 8 hours and then stirred at 80° C. for 4 hours. The reaction mixture was poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 0.66 g of 4-chloro-2-methyl-6-(3-methylphenyl)pyrimidine.

### 4-chloro-2-methyl-6-(3-methylphenyl)pyrimidine

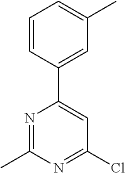

¹H-NMR: 2.45(s,3H), 2.78(s,3H), 7.34(d,1H), 7.40(t,1H), 7.54(s,1H), 7.83(d,1H), 7.88(s,1H)

### Reference Production Example 59

To 6 ml of N,N-dimethylformamide, 0.66 g of 4-chloro-2-methyl-6-(3-methylphenyl)pyrimidine, 0.71 g of zinc cyanide and 0.11 g of tetrakis(triphenylphosphine)palladium were added. This mixture was stirred at 90° C. for 5 hours and then stirred at 100° C. for 6 hours. The reaction mixture was left standing to cool, poured into water and then extracted three times with tert-butyl methyl ether. The organic layer was washed with saturated brine, dried over anhydrous magnesium sulfate and then concentrated to obtain a crude product. This crude product was used in the next step without purification.

To 5 ml of ethanol, 0.35 g of sodium hydrogen carbonate and 0.29 g of hydroxylamine hydrochloride were added. This mixture was heated under reflux for 1 hour. After cooling this mixture to 0° C., the crude product was added to this mixture. This mixture was stirred at room temperature for 20 minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.3 g of 2-methyl-6-(3-methylphenyl)pyrimidine-4-carboxamide oxime.

### 2-methyl-6-(3-methylphenyl)pyrimidine-4-carboxamide oxime

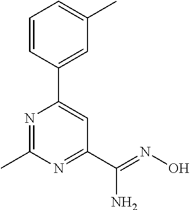

¹H-NMR (DMSO-d₆): 2.42(s,3H), 2.73(s,3H), 5.96(s,2H), 7.37(d,1H), 7.44(t,1H), 7.93(d,1H), 7.97(s,1H), 8.05(s,1H), 10.42 (s, 1H)

### Reference Production Example 60

To 12 ml of 1,4-dioxane, 0.86 g of 4-fluorophenylboronic acid, 1.7 g of potassium carbonate, 0.13 g of dichlorobis(triphenylphosphine)palladium and 1 g of 4,6-dichloro-2-methylpyrimidine were added. This mixture was stirred at 60° C. for 14 hours. The reaction mixture was poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 0.7 g of 4-chloro-6-(4-fluorophenyl)-2-methylpyrimidine.

### 4-chloro-6-(4-fluorophenyl)-2-methylpyrimidine

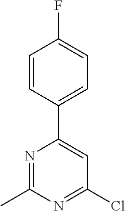

¹H-NMR: 2.77(s,3H), 7.17-7.22(m,2H), 7.52(s,1H), 8.06-8.10 (m, 2H)

### Reference Production Example 61

To a mixture of 1 ml of water, 0.14 g of sodium cyanide and 0.03 g of 1,4-diazabicyclo[2.2.2]octane, 3 ml of dimethyl sulfoxide and 0.52 g of 4-chloro-6-(4-fluorophenyl)-2-methylpyrimidine were added at 0° C. This mixture was stirred at 40° C. for 4 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.5 g of 6-(4-fluorophenyl)-2-methylpyrimidine-4-carbonitrile.

### 6-(4-fluorophenyl)-2-methylpyrimidine-4-carbonitrile

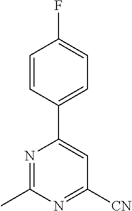

¹H-NMR: 2.84(s,3H), 7.20-7.26(m,2H), 7.79(s,1H), 8.11-8.15 (m, 2H)

### Reference Production Example 62

To 4 ml of ethanol, 0.31 g of sodium hydrogen carbonate and 0.25 g of hydroxylamine hydrochloride were added. This mixture was heated under reflux for 1 hour. After cooling this mixture to 0° C., 0.39 g of 6-(4-fluorophenyl)-2-methylpyrimidine-4-carbonitrile was added to this mixture. This mixture was stirred at room temperature for 30 minutes.

The reaction mixture was concentrated and water was added to the residue, followed by extraction three times with ethyl acetate. The organic layer was washed with saturated brine, dried over anhydrous magnesium sulfate and then concentrated. The residue was washed with hexane to obtain 0.42 g of 6-(4-fluorophenyl)-2-methylpyrimidine-4-carboxamide oxime.

### 6-(4-fluorophenyl)-2-methylpyrimidine-4-carboxamide oxime

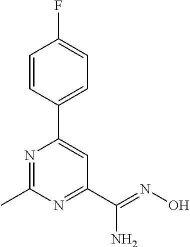

¹H-NMR (DMSO-d₆): 2.72(s,3H), 5.96(s,2H), 7.35-7.40(m,2H), 8.06(s,1H), 8.20-8.25(m,2H), 10.33(s,1H)

### Reference Production Example 63

To 4 ml of N,N-dimethylformamide, 0.47 g of 4-chloro-2-methyl-6-(2-methylphenyl)pyrimidine, 0.5 g of zinc cyanide and 0.07 g of tetrakis(triphenylphosphine)palladium were added. This mixture was stirred at 90° C. for 2 hours and then stirred at 120° C. for 4 hours. The reaction mixture was left standing to cool, poured into water and then extracted three times with tert-butyl methyl ether. The organic layer was washed with saturated brine, dried over anhydrous magnesium sulfate and then concentrated to obtain a crude product. This crude product was used in the next step without purification.

To 4 ml of ethanol, 0.26 g of sodium hydrogen carbonate and 0.21 g of hydroxylamine hydrochloride were added. This mixture was heated under reflux for 1 hour. After cooling this mixture to 0° C., the crude product was added to this mixture. This mixture was stirred at room temperature for 30 minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.26 g of 2-methyl-6-(2-methylphenyl)pyrimidine-4-carboxamide oxime.

### 2-methyl-6-(2-methylphenyl)pyrimidine-4-carboxamide oxime

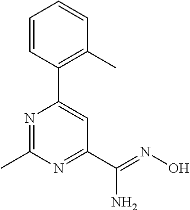

¹H-NMR (DMSO-d₆): 2.36(s,3H), 2.71(s,3H), 5.96(s,2H), 7.31-7.42(m,3H), 7.47(d,1H), 7.70(s,1H), 10.34(s,1H)

### Reference Production Example 64

0.29 g of sodium hydride (60% in oil) was suspended in 12 ml of tetrahydrofuran and 0.66 g of benzyl alcohol was added at 0° C. This mixture was stirred at 20 minutes. To this mixture, 1 g of 4,6-dichloro-2-methylpyrimidine was added. This mixture was further stirred for 40 minutes. The reaction mixture was poured into an aqueous saturated ammonium chloride solution and then extracted three times with tert-butyl methyl ether. The organic layer was washed with saturated brine, dried over anhydrous magnesium sulfate and then concentrated. The residue was subjected to silica gel column chromatography to obtain 1.1 g of 4-benzyloxy-6-chloro-2-methylpyrimidine.

### 4-benzyloxy-6-chloro-2-methylpyrimidine

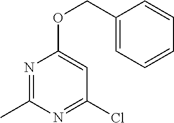

¹H-NMR: 2.62(s,3H), 5.42(s,2H), 6.62(s,1H), 7.32-7.44(m,5H)

### Reference Production Example 65

To 7 ml of N,N-dimethylformamide, 0.74 g of 4-benzyloxy-6-chloro-2-methylpyrimidine, 0.77 g of zinc cyanide and 0.11 g of tetrakis(triphenylphosphine)palladium were added. This mixture was stirred at 90° C. for 3 hours and then stirred at 100° C. for 3 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.47 g of 4-benzyloxy-2-methylpyrimidine-6-carbonitrile.

### 4-benzyloxy-2-methylpyrimidine-6-carbonitrile

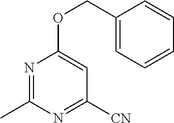

¹H-NMR: 2.67(s,3H), 5.46(5,2H), 6.93(s,1H), 7.35-7.45(m,5H)

### Reference Production Example 66

To 5 ml of ethanol, 0.35 g of sodium hydrogen carbonate and 0.29 g of hydroxylamine hydrochloride were added. This mixture was heated under reflux for 1 hour. After cooling this mixture to 0° C., 0.47 g of 4-benzyloxy-2-methylpyrimidine-6-carbonitrile was added to this mixture. This mixture was stirred at room temperature for 30 minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.45 g of 6-benzyloxy-2-methylpyrimidine-4-carboxamide oxime.

### 6-benzyloxy-2-methylpyrimidine-4-carboxamide oxime

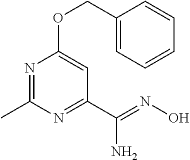

¹H-NMR (DMSO-d₆): 2.58(s,3H), 5.42(s,2H), 5.85(s,2H), 6.99(s,1H), 7.32-7.49(m,5H), 10.19(s,1H)

### Reference Production Example 67

To 5 ml of methanol, 4 g of sodium methoxide (28% methanol solution), 0.75 g of cyclopropanecarboxamidine hydrochloride and 1 g of benzoylacetic acid ethyl ester were added. This mixture was heated under reflux for 20 hours. The reaction mixture was left standing to cool and then concentrated. To the residue, 10% hydrochloric acid was added, followed by extraction three times with ethyl acetate. The organic layer was washed with saturated brine, dried over anhydrous magnesium sulfate and then concentrated. The residue was washed with hexane to obtain 0.39 g of 2-cyclopropyl-6-phenylpyrimidin-4-ol.

### 2-cyclopropyl-6-phenylpyrimidin-4-ol

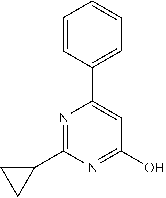

¹H-NMR (DMSO-d₆): 1.04-1.16(m,4H), 1.93-1.99(m,1H), 6.64(s,1H), 7.43-7.47(m,3H), 7.94-7.99(m,2H), 12.67(bs,1H)

### Reference Production Example 68

To a mixture of 4 ml of toluene, 0.39 g of 2-cyclopropyl-6-phenylpyrimidin-4-ol and 0.03 g of N,N-dimethylformamide, 0.33 g of thionyl chloride was slowly added dropwise at 80° C. This mixture was stirred at 80° C. for 3 hours. The reaction mixture was left standing to cool to room temperature, poured into water and then extracted three times with tert-butyl methyl ether. The organic layer was washed in turn with an aqueous saturated sodium hydrogen carbonate solution and saturated brine, dried over anhydrous magnesium sulfate and then concentrated. The residue was subjected to silica gel column chromatography to obtain 0.35 g of 4-chloro-2-cyclopropyl-6-phenylpyrimidine.

### 4-chloro-2-cyclopropyl-6-phenylpyrimidine

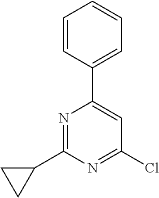

¹H-NMR: 1.10-1.14(m,2H), 1.24-1.28(m,2H), 2.27-2.34(m,1H), 7.47-7.52(m,4H), 8.02-8.06(m,2H)

### Reference Production Example 69

To a mixture of 0.5 ml of water, 0.06 g of sodium cyanide and 0.01 g of 1,4-diazabicyclo[2.2.2]octane, 1 ml of dimethyl sulfoxide and 0.52 g of 4-chloro-2-cyclopropyl-6-phenylpyrimidine were added at 0° C. This mixture was stirred at 40° C. for 3 hours. The reaction mixture was poured into water and then extracted three times with tert-butyl methyl ether. The organic layer was washed with saturated brine, dried over anhydrous magnesium sulfate and then concentrated to obtain a crude product. This crude product was used in the next step without purification.

To 2 ml of ethanol, 0.16 g of sodium hydrogen carbonate and 0.13 g of hydroxylamine hydrochloride were added. This mixture was heated under reflux for 1 hour. After cooling this mixture to 0° C., the crude product was added to this mixture. This mixture was stirred at room temperature for 30 minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.16 g of 2-cyclopropyl-6-phenylpyrimidine-4-carboxamide oxime.

### 2-cyclopropyl-6-phenylpyrimidine-4-carboxamide oxime

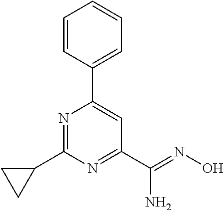

¹H-NMR (DMSO-d₆): 1.07-1.21(m,4H), 2.27-2.34(m,1H), 5.96(s,2H), 7.53-7.58(m,3H), 7.99(3,1H), 8.11-8.15(m,2H), 10.31(s,1H)

### Reference Production Example 70

To 11 ml of 1,4-dioxane, 0.7 g of phenylboronic acid, 1.56 g of potassium carbonate, 0.12 g of dichlorobis(triphenylphosphine)palladium and 1 g of 4,6-dichloro-2,5-dimethylpyrimidine were added. This mixture was stirred at 60° C. for 3 hours and then stirred at 100° C. for 4 hours. The reaction mixture was left standing to cool to room temperature, poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 0.69 g of 4-chloro-2,5-dimethyl-6-phenylpyrimidine.

### 4-chloro-2,5-dimethyl-6-phenylpyrimidine

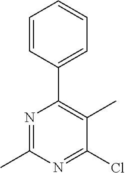

¹H-NMR: 2.37(s,3H), 2.72(s,3H), 7.44-7.54(m,5H)

### Reference Production Example 71

To 6 ml of N,N-dimethylformamide, 0.55 g of 4-chloro-2,5-dimethyl-6-phenylpyrimidine, 0.59 g of zinc cyanide and 0.09 g of tetrakis(triphenylphosphine)palladium were added. This mixture was stirred at 90° C. for 2 hours and then stirred at 120° C. for 4 hours. The reaction mixture was left standing to cool, poured into water and then extracted three times with text-butyl methyl ether. The organic layer was washed with saturated brine, dried over anhydrous magnesium sulfate and then concentrated. The residue was subjected to silica gel column chromatography to obtain 0.24 g of 2,5-dimethyl-6-phenylpyrimidine-4-carbonitrile.

### 2,5-dimethyl-6-phenylpyrimidine-4-carbonitrile

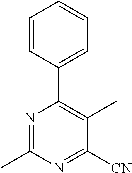

¹H-NMR: 2.55(s,3H), 2.79(s,3H), 7.51-7.59(m,5H)

### Reference Production Example 72

To 3 ml of ethanol, 0.19 g of sodium hydrogen carbonate and 0.16 g of hydroxylamine hydrochloride were added. This mixture was heated under reflux for 1 hour. After cooling this mixture to 0° C., 0.24 g of 2,5-dimethyl-6-phenylpyrimidine-4-carbonitrile was added to this mixture. This mixture was stirred at room temperature for 30 minutes. The reaction mixture was concentrated and water was added to the residue, followed by extraction three times with ethyl acetate. The organic layer was washed with saturated brine, dried over anhydrous magnesium sulfate and then concentrated. The residue was washed with hexane to obtain 0.24 g of 2,5-dimethyl-6-phenylpyrimidine-4-carboxamide oxime. 2,5-dimethyl-6-phenylpyrimidine-4-carboxamide oxime

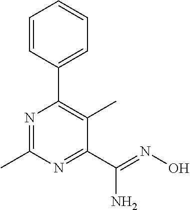

¹H-NMR (DMSO-d₆): 2.37(s,3H), 2.64(s,3H), 5.94(s,2H), 7.48-7.57 (m, 5H), 10.12 (s, 1H)

### Reference Production Example 73

To 12 ml of 1,4-dioxane, 1.12 g of phenylboronic acid, 1.86 g of potassium carbonate, 0.22 g of dichlorobis(triphenylphosphine)palladium and 1 g of 4,6-dichloro-5-methylpyrimidine were added. This mixture was stirred at 70° C. for 5 hours. The reaction mixture was poured into an aqueous saturated ammonium chloride solution and then extracted three times with ethyl acetate. The organic layer was washed with saturated brine, dried over anhydrous magnesium sulfate and then concentrated. The residue was subjected to silica gel column chromatography to obtain 0.9 g of 4-chloro-5-methyl-6-phenylpyrimidine.

### 4-chloro-5-methyl-6-phenylpyrimidine

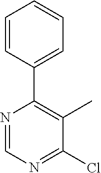

¹H-NMR: 2.44(s,3H), 7.49-7.57(m,5H), 8.88(s,1H)

### Reference Production Example 74

To 10 ml of N,N-dimethylformamide, 0.92 g of 4-chloro-5-methyl-6-phenylpyrimidine, 1.06 g of zinc cyanide, 0.06 g of zinc bromide, 0.16 g of tetrakis(triphenylphosphine)palladium and one drop of water were added. This mixture was stirred at 110° C. for 9 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.55 g of 5-methyl-6-phenylpyrimidine-4-carbonitrile.

### 5-methyl-6-phenylpyrimidine-4-carbonitrile

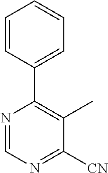

¹H-NMR: 2.64 (s, 3H), 7.49-7.56(s,3H), 7.58-7.62(m,2H), 9.21(s,1H)

### Reference Production Example 75

To 5 ml of ethanol, 0.44 g of sodium hydrogen carbonate and 0.37 g of hydroxylamine hydrochloride were added. This mixture was heated under reflux for 1 hour. After cooling this mixture to 0° C., 0.55 g of 5-methyl-6-phenylpyrimidine-4-carbonitrile was added to this mixture. This mixture was stirred at room temperature for 3D minutes. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.56 g of 5-methyl-6-phenylpyrimidine-4-carboxamide oxime.

### 5-methyl-6-phenylpyrimidine-4-carboxamide oxime

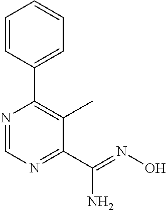

¹H-NMR (DMSO-d₆): 2.43(s,3H), 5.98(s,2H), 7.50-7.54(m,3H), 7.58-7.61(m,2H), 9.09(s,1H), 10.18(s,1H)

### Reference Production Example 76

To 12 ml of tetrahydrofuran, 4,6-dichloro-2-methyipyrimidine was added and 6.1 ml of benzylmagnesium bromide (1M tetrahydrofuran solution) was added dropwise at −78° C. This mixture was stirred for 24 hours while slowly heating to room temperature. An aqueous saturated ammonium chloride solution was poured into the reaction mixture, followed by extraction three times with tert-butyl methyl ether. The organic layer was washed with saturated brine. The organic layer was dried over anhydrous magnesium sulfate and then concentrated. The residue was subjected to silica gel column chromatography to obtain 0.47 g of 6-benzyl-4-chloro-2-methylpyrimidine.

### 6-benzyl-4-chloro-2-methylpyrimidine

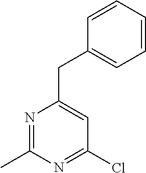

¹H-NMR: 2.71(s,3H), 4.06(s,2H), 6.89(s,1H), 7.24-7.36(m,5H)

### Reference Production Example 77

To 10 ml of methanol, 12.5 g of sodium methoxide (28% methanol solution), 5 g of acetoamidine hydrochloride and 2.5 g of 3-oxo-3-(2-pyridyl)propanoic acid ethyl ester were added. This mixture was heated under reflux for 20 hours. The reaction mixture was left standing to cool and then concentrated. To the residue, 10% hydrochloric acid was added. A precipitated crystal was collected by filtration and then washed with water and hexane to obtain 1.7 g of 2-methyl-6-(2-pyridyl)pyrimidin-4-ol.

### 2-methyl-6-(2-pyridyl)pyrimidin-4-ol

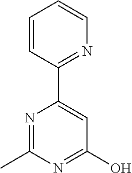

¹H-NMR (DMSO-d₆): 2.39(s,3H), 7.06(s,1H), 7.48-7.50(m,1H), 7.93-7.98(m,1H), 8.26(d,1H), 8.68-8.70(m,1H), 12.57(bs,1H)

### Reference Production Example 78

To 3 ml of phosphorus oxychloride, 1.7 g of 2-methyl-6-(2-pyridyl)pyrimidin-4-ol and 2.3 g of N,N-dimethylaniline were added. This mixture was stirred at 100° C. for 6 hours. The reaction mixture was left standing to cool, slowly poured into water and then extracted three times with ethyl acetate. The organic layer was washed in turn with an aqueous saturated sodium hydrogen carbonate solution and saturated brine, dried over anhydrous magnesium sulfate and then concentrated. The residue was subjected to silica gel column chromatography to obtain 1.1 g of 4-chloro-2-methyl-6-(2-pyridyl)pyrimidine.

### 4-chloro-2-methyl-6-(2-pyridyl)pyrimidine

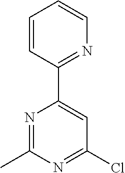

¹H-NMR: 2.80(s,3H), 7.46(dd,1H), 7.59(s,1H), 8.37-8.40(m,1H), 8.76(dd,1H), 9.25(d,1H)

### Reference Production Example 79

To a mixture of 1.5 ml of water, 0.2 g of sodium cyanide and 0.07 g of 1,4-diazabicyclo[2.2.2]octane, 4.5 ml of dimethyl sulfoxide and 0.6 g of 4-chloro-2-methyl-6-(2-pyridyl)pyrimidine were added at 0° C. This mixture was stirred at 50° C. for 5 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.44 g of 2-methyl-6-(2-pyridyl)pyrimidine-4-carbonitrile.

### 2-methyl-6-(2-pyridyl)pyrimidine-4-carbonitrile

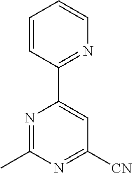

¹H-NMR: 2.86(s,3H), 7.46-7.47(m,1H), 7.88-7.92(m,1H), 8.52-8.54(m,2H), 8.73-8.75(m,1H)

### Reference Production Example 80

To 7 ml of N,N-dimethylformamide, 0.79 g of 4-chloro-2-isopropyl-6-phenylpyrimidine, 0.8 g of zinc cyanide, 0.05 g of zinc bromide, 0.05 g of tetrakis(triphenylphosphine)palladium, 0.01 g of zinc and one drop of water were added. This mixture was slowly heated from 100° C. to 140° C. while stirring and stirred at 140° C. for 7 hours. The reaction mixture was left standing to cool, poured into water and then extracted three times with tert-butyl methyl ether. The organic layer was washed with saturated brine, dried over anhydrous magnesium sulfate and then concentrated. The residue was subjected to silica gel column chromatography to obtain 0.12 g of 2-isopropyl-6-phenylpyrimidine-4-carbonitrile.

### 2-isopropyl-6-phenylpyrimidine-4-carbonitrile

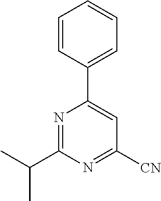

¹H-NMR: 1.41(d,6H), 3.29-3.36(m,1H), 7.52-7.58(m,3H), 7.83(s,1H), 8.14(d,2H)

### Reference Production Example 81

To 2 ml of ethanol, 0.08 g of sodium hydrogen carbonate and 0.07 g of hydroxylamine hydrochloride were added. This mixture was heated under reflux for 1 hour. After cooling this mixture to 0° C., 0.16 g of 2-isopropyl-6-phenylpyrimidine-4-carbonitrile was added to this mixture. This mixture was stirred at room temperature for 2 hours and a half. The reaction mixture was concentrated. To the residue, water was added, followed by extraction three times with ethyl acetate. The organic layer was washed with saturated brine, dried over anhydrous magnesium sulfate and then concentrated. The residue was washed with hexane to obtain 0.14 g of 2-isopropyl-6-phenylpyrimidine-4-carboxamide oxime.

### 2-isopropyl-6-phenylpyrimidine-4-carboxamide oxime

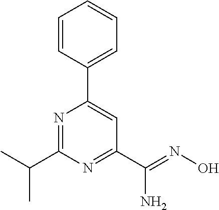

¹H-NMR (DMSO-d₆): 1.37(d,6H), 3.20-3.27(m,1H), 5.99(s,2H), 7.55-7.58 (m, 3H), 8.07 (s, 1H), 8.16-8.18 (m, 2H), 10.32 (s,1H)

Formulation Examples will be shown below. Parts are by weight.

### Formulation Example 1

10 parts of each of the present compounds (1) to (56) is dissolved in a mixture of 35 parts of xylene and 35 parts of N,N-dimethylformamide, and 14 parts of polyoxyethylene styryl phenyl ether and 6 parts of calcium dodecylbenzenesulfonate are added thereto, followed by well stirring and mixing, to give 10% emulsifiable concentrates for each compound.

### Formulation Example 2

20 parts of each of the present compounds (1) to (56) is added to a mixture containing 4 parts of sodium lauryl sulfate, 2 parts of calcium lignin sulfonate, 20 parts of a synthetic hydrated silicon oxide fine powder and 54 parts of diatomaceous earth, followed by well stirring and mixing, to give 20% wettable powders for each compound.

### Formulation Example 3

To 2 parts of each of the present compounds (1) to (56) are added 1 part of a synthetic hydrated silicon oxide fine powder, 2 parts of calcium lignin sulfonate, 30 parts of bentonite and 65 parts of kaolin clay, followed by well stirring and mixing, and an appropriate amount of water is added to this mixture, followed by further stirring, granulation with a granulator, and air drying, to give 2% granules for each compound.

### Formulation Example 4

1 part of each of the present compounds (1) to (56) is dissolved in an appropriate amount of acetone, 5 parts of a synthetic hydrated silicon oxide fine powder, 0.3 parts of PAP, and 93.7 parts of Fubasami clay are added thereto, followed by well stirring and mixing, and acetone is removed by evaporation from the mixture, to give 1% powders for each compound.

### Formulation Example 5

10 parts of each of the present compounds (1) to (56), 35 parts of white carbon containing 50 parts of a polyoxyethylene alkyl ether sulfate ammonium salt, and 55 parts of water are mixed and pulverized by a wet grinding method to give 10% flowable formulations for each compound.

### Formulation Example 6

0.1 parts of each of the present compounds (1) to (56) is dissolved in a mixture of 5 parts of xylene and 5 parts of trichloroethane, and the resulting solution is mixed with 89.9 parts of deodorized kerosine to give 0.1% oil solutions for each compound.

### Formulation Example 7

10 mg of each of the present compounds (1) to (56) is dissolved in 0.5 ml of acetone, the solution is applied to 5 g of powdery solid animal food (powdery solid animal food for bleeding CE-2; a product of CLEA Japan, Inc.) and mixed uniformly, and acetone is removed by evaporation from the mixture, to give poison baits for each compound.

### Formulation Example 8

0.1 parts of each of the present compounds (1) to (56) and 49.9 parts of NEO-CHIOZOL (CHUO KASEI Co., LTD) are charged into an aerosol can, and an aerosol valve is fixed to the can. Then 25 parts of dimethyl ether and 25 parts of LPG are charged into the can, followed by shaking it and fitting an actuator on it to give an oil aerosol.

### Formulation Example 9

0.6 parts of each of the present compounds (1) to (56), 0.01 parts of BHT, 5 parts of xylene, 3.39 parts of deodorized kerosene and 1 part of an emulsifier [Atmos 300 (registered trade name for ATMOS CHEMICAL LTD)] are mixed and dissolved. The solution obtained and 50 parts of distilled water are charged into an aerosol container, and a valve is fixed to the container. 40 Parts of propellant (LPG) are charged thereinto under pressure through the valve to give an aqueous aerosol.

Pest control activity of the present compound will be shown below by Test Examples.

### Test Example 1

The formulation of each of the present compounds (1), (7), (9) to (13), (15), (17) to (19), (22), (24), (27) to (30), (33) to (36), (48) and (49) obtained in Formulation Example 5 was diluted with water so as to adjust the concentration of the active ingredient to 500 ppm to prepare a test spray solution.

Thirty imagines of Aphis gossypii were parasitized on a cucumber seedling at 1st true leaf stage placed in a polyethylene cup. One day after parasitizing, 20 ml (per cup) of the test spray solution was sprayed over the cucumber seedling. Six days after spraying, the number of the surviving worms of Aphis gossypii was examined and a control value was determined by the following equation.

Control value (%)={1−(Cb×Tai)/(Cai×Tb))×100

wherein symbols have the following meanings.
Cb: Number of worms in non-treated section before treatment
Cai: Number of worms in a non-treated section on observation
Tb: Number of worms in treated section before treatment
Tai: Number of worms in treated section on observation

As a result, the treated section of the test spray solutions of any present compound exhibited the control value of 60% or more.

### Test Example 2

The formulation of each of the present compounds (1), (3), (5), (7) to (11), (13), (14), (18), (20), (22), (24), (26), (27), (29), (30) and (33) to (35) obtained in Formulation Example 1 was diluted with water so as to adjust the concentration of the active ingredient to 500 ppm to prepare a test spray solution.

About sixty female imagines of Tetranychus urticae were released on brush bean (Phaseolus vulgaris) in the primary leaf stage, which had been potted in a polyethylene cup for 7 days after the seeding. One day after release of female imagines of Tetranychus urticae, 30 ml (per cup) of the test spray solution was sprayed over the brush bean seedling.

On the 8th and 13th day after spraying, the numbers of survival Tetranychus urticae on the leaf of the brush bean plant were examined, and the control rate was calculated by the following equation.

Control rate=100×{1−(number of survival Tetranychus urticae in treated section)/(number of survival Tetranychus urticae in non-treated section)}

As a result, the section treated with any present compound exhibited the control rate of 90% or more on 8th day and 13th day after the treatment.

A compound represented by formula (C) (described in US2002/0013326, hereinafter referred to as a comparative compound (C))

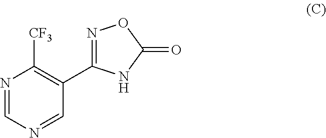

was tested under the same conditions as in Test Example 2. As a result, the treated section treated with a test spray solution of the comparative compound (C) exhibited the control value of less than 30%.

### INDUSTRIAL APPLICABILITY

The present compound has excellent control activity against pests and is useful as an active ingredient of a pest controlling agent.

1. A pyrimidine compound represented by formula (I): 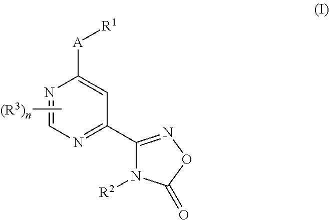
wherein R¹ represents a C1-C7 haloalkyl group optionally substituted with one or more members selected from Group γ, a phenyl group optionally substituted with one or more members selected from Group β, or a pyridyl group optionally substituted with one or more members selected from Group β;
A represents a single bond, oxygen, sulfur, —N(R¹⁰)—, —CH₂—, or —CH₂O—;
wherein R¹⁰ represents a C1-C7 alkyl group optionally substituted with halogen, a C3-C7 alkenyl group optionally substituted with halogen, a C3-C7 alkynyl group optionally substituted with halogen, a C2-C7 alkoxyalkyl group, a cyanomethyl group, or hydrogen;
R² represents a C1-C7 alkyl group optionally substituted with halogen, a (C3-C7 cycloalkyl)methyl group optionally substituted with one or more members selected from Group α, a benzyl group optionally substituted with one or more members selected from Group β, a cyanomethyl group hydrogen, or
any one of the following groups Q¹ to Q⁵: 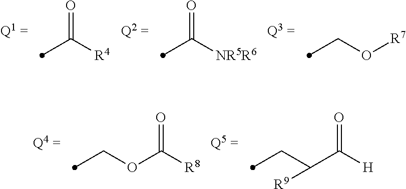
wherein R⁴ represents a C1-C7 alkyl group optionally substituted with halogen, a C3-C7 cycloalkyl group optionally substituted with one or more members selected from Group α, or hydrogen,
R⁵ represents a C1-C7 alkyl group optionally substituted with halogen, a C3-C7 alkenyl group optionally substituted with halogen, a C1-C7 alkyloxy group optionally substituted with halogen, or a C3-C7 cycloalkyl group optionally substituted with one or more members selected from Group α, R⁶ represents a C1-C7 alkyl group optionally substituted with halogen, a C3-C7 alkenyl group optionally substituted with halogen, a C3-C7 cycloalkyl group optionally substituted with one or more members selected from Group α, or hydrogen, or
—NR⁵R⁶ is replaced by a pyrrolidin-1-yl group optionally substituted with one or more members selected from Group α, a piperidino group optionally substituted with one or more members selected from Group α, a hexamethyleneimin-1-yl group optionally substituted with one or more members selected from Group α, a morpholino group optionally substituted with one or more members selected from Group α, or a thiomorpholin-4-yl group optionally substituted with one or more members selected from Group α,
R⁷ represents a C1-C7 alkyl group optionally substituted with halogen, a phenyl group optionally substituted with one or more members selected from Group β, a benzyl group optionally substituted with one or more members selected from Group β, or a C3-C7 cycloalkyl group optionally substituted with one or more members selected from Group α,
R⁸ represents a C1-C7 alkyl group optionally substituted with halogen, a phenyl group optionally substituted with one or more members selected from Group β, or a C3-C7 cycloalkyl group optionally substituted with one or more members selected from Group α, and
R⁹ represents a C1-C7 alkyl group optionally substituted with halogen, or hydrogen;
R³ represents a C1-C7 alkyl group optionally substituted with halogen, a C1-C7 alkylthio group optionally substituted with halogen, a C1-C7 alkyloxy group optionally substituted with halogen, a C3-C7 cycloalkyl group optionally substituted with one or more members selected from Group α, a C3-C7 cycloalkyloxy group optionally substituted with one or more members selected from Group α, or halogen; and
n represents an integer of 0 to 2 and, when n is 2, each R³ is the same or different:
Group γ: a group consisting of a C1-C3 alkyloxy group optionally substituted with halogen, a C3-C7 alkenyloxy group optionally substituted with halogen, a C3-C7 alkynyloxy group optionally substituted with halogen, and a tri(C1-C4 alkyl)silyloxy group and a hydroxy group;
Group α: a group consisting of a C1-C7 alkyl group optionally substituted with halogen, and halogen; and
Group β: a group consisting of a C1-C7 alkyl group optionally substituted with halogen, a C1-C7 alkyloxy group optionally substituted with halogen, halogen, a cyano group, and a nitro group.

2. The pyrimidine compound according to claim 1, wherein R² is hydrogen.

3. The pyrimidine compound according to claim 1, wherein R² is Q¹.

4. The pyrimidine compound according to claim 1, wherein R² is Q².

5. The pyrimidine compound according to claim 1, wherein R² is Q³.

6. The pyrimidine compound according to claim 1, wherein R² is Q⁴.

7. The pyrimidine compound according to claim 1, wherein R¹ is a C1-C7 haloalkyl group optionally substituted with one or more members selected from Group γ.

8. The pyrimidine compound according to claim 1, wherein R¹ is a phenyl group optionally substituted with one or more members selected from Group β, or a pyridyl group optionally substituted with one or more members selected from Group β.

9. A pest controlling agent comprising the pyrimidine compound according to claim 1 and an inert carrier.

10. A method for controlling pests, which comprises applying an effective amount of the pyrimidine compound according to claim 1 to pests or habitats of pests.

11. Use of the pyrimidine compound according to claim 1 for control of pests.
